(19) United States
(12) Patent Application Publication
(10) Pub. No.: US 2025/0114470 A1
(43) Pub. Date: Apr. 10, 2025

(54) THERAPY FOR POLYCYSTIC KIDNEY DISEASE

(71) Applicant: University of Massachusetts, Westborough, MA (US)

(72) Inventors: Gregory J. Pazour, Framingham, MA (US); Abigail O. Smith, Belmont, MA (US); William Tyler Frantz, New Haven, CT (US); Julie A. Jonassen, Belchertown, MA (US); Kenley M. Preval, Shrewsbury, MA (US)

(21) Appl. No.: 18/912,456

(22) Filed: Oct. 10, 2024

Related U.S. Application Data
(60) Provisional application No. 63/589,316, filed on Oct. 10, 2023.

(51) Int. Cl. A61K 47/68; A61K 31/55; A61K 48/00; A61P 13/12; C12N 15/113

(52) U.S. Cl. CPC A61K 47/6849; A61K 31/55; A61K 47/6803; A61K 47/6817; A61K 48/0033; A61P 13/12; C12N 15/113; C12N 2310/11; C12N 2310/14

(57) ABSTRACT

The disclosure herein provides, in example embodiments, methods of treating a subject in need thereof, e.g., a subject with polycystic kidney disease (PKD), for example, with an antibody-drug conjugate comprising, e.g., tumor-associated calcium signal transducer 2 protein (TACSTD2).

### RELATED APPLICATIONS

This application claims the benefit of U.S. Provisional Application No. 63/589,316, filed Oct. 10, 2023. The entire teachings of the above application are incorporated herein by reference.

### GOVERNMENT SUPPORT

This invention was made with government support under DK103632 and GM060992 from National Institutes of Health. The government has certain rights in the invention.

### INCORPORATION BY REFERENCE OF MATERIAL IN XML

This application incorporates by reference the Sequence Listing contained in the following extensible Markup Language (XML) file being submitted concurrently herewith:
a) File name: 54391037001_Sequence_Listing.xml; created Oct. 10, 2024, 4,609 Bytes in size.

### BACKGROUND

Polycystic kidney disease (PKD) is an important cause of end stage renal disease, but treatment options are limited. While later stages of the disease have been extensively studied, mechanisms driving the initial conversion of a renal tubule into a cyst are not understood.

Autosomal dominant polycystic kidney disease (ADPKD) patients lose kidney function as they age and about half reach end stage renal disease by late adulthood, requiring dialysis or kidney transplant. The only FDA-approved drug therapy for ADPKD provides modest benefits accompanied by significant side effects. Furthermore, there are no FDA-approved drug therapies for ADPKD in adolescents, children, or infants.

Accordingly, there is a need for additional, improved, and age-inclusive therapies for ADPKD.

### SUMMARY

There is a critical need to develop methods for treating polycystic kidney disease (PKD), including autosomal dominant polycystic kidney disease (ADPKD), autosomal recessive polycystic kidney disease (ARPKD), and PKD disorders affecting adults, adolescents, children, or infants. The disclosure provides such methods and treatments.

In one aspect, the disclosure herein provides a method of treating a subject in need thereof, comprising administering to the subject a therapeutically effective amount of an antibody-therapeutic conjugate, wherein the antibody-therapeutic conjugate comprises an antibody that binds a marker of cystic epithelium, and a therapeutic drug; and wherein the subject has or is predisposed to have a polycystic kidney disease (PKD).

In some embodiments, the antibody is a whole antibody or antigen-binding fragment thereof.

In some embodiments, the marker of cystic epithelium comprises tumor associated calcium signal transducer 2 (TACSTD2).

In some embodiments, the antibody is selected from the group comprising sacituzumab, datopotamab, and humanized anti-TACSTD2 antibody RN926.

In some embodiments, the antibody binds TACSTD2 and an additional antigen.

In some embodiments, the therapeutic drug comprises a cytotoxic drug, a cytostatic drug, a gene therapy, a protein, or a combination thereof.

In some embodiments, the gene therapy provides a means for gene editing, gene augmentation, gene replacement, gene addition or a combination thereof.

In some embodiments, the gene therapy targets at least one gene selected from polycystin 1, transient receptor potential channel interacting (PKD1), polycystin 2, transient receptor potential cation channel (PKD2), and tumor associated calcium signal transducer 2 (TACSTD2).

In some embodiments, the gene therapy comprises an anti-sense oligonucleotide (ASO), a small interfering RNA (siRNA), a viral vector, a non-viral delivery system or a combination thereof.

In some embodiments, the therapeutic drug comprises a topoisomerase I inhibitor, a tubulin polymerization inhibitor, a vasopressin receptor 2 antagonist, an mTOR inhibitor or a combination thereof.

In some embodiments, the therapeutic drug comprises a topoisomerase I inhibitor SN38, deruxtecan, tubulin polymerization inhibitor Aur0101, tolvaptan, rapamycin or a combination thereof.

In some embodiments, the antibody-therapeutic conjugate comprises sacituzumab govitecan (hRS7-SN38), datopotamab deruxtecan (Dato-DXd), PF-06664178 (RN926-Aur0101) or a combination thereof.

In some embodiments, the polycystic kidney disease is autosomal dominant polycystic kidney disease (ADPKD) or autosomal recessive polycystic disease (ARPKD).

In some embodiments, the ADPKD is associated with PKD1 or PKD2.

In some embodiments, the method further comprises administering a therapeutically effective amount of an additional therapeutic agent or a prophylactic agent to the subject.

In some embodiments, the additional therapeutic agent or the prophylactic agent is tolvaptan.

In some embodiments, the method further comprises administering an additional treatment to the subject.

In another aspect, the disclosure herein provides a method of evaluating a biological response to an antibody-therapeutic conjugate, the method comprising: a) exposing: i) a first kidney organoid to the antibody-therapeutic conjugate without exposing a second kidney organoid to the antibody-therapeutic conjugate; or ii) a first population of stem cells to the antibody-therapeutic conjugate, thereby producing a population of exposed stem cells, without exposing a second population of cells to the antibody-therapeutic conjugate, and differentiating the population of exposed stem cells into a first kidney organoid and differentiating the second population of stem cells into a second kidney organoid; and b) comparing the first kidney organoid and the second kidney organoid, thereby evaluating the biological response.

In some embodiments, the stem cells are induced pluripotent stem (IPS) cells generated from a biological sample from a subject.

In yet another aspect, the disclosure herein provides a method of reducing or ablating cystic kidney tissue in a subject in need thereof, the method comprising administering to the subject a therapeutically effective amount of an antibody-therapeutic conjugate, wherein said antibody-therapeutic conjugate comprises: a) a monospecific antibody or antigen-binding fragment thereof that binds TACSTD2 or a bispecific antibody or antigen-binding fragment thereof that binds TACSTD2 and a second antigen, and b) a topoisomerase I inhibitor, a tubulin polymerization inhibitor, a vasopressin receptor 2 antagonist, an mTOR inhibitor or a combination thereof.

In yet another aspect, the disclosure herein provides antibody-therapeutic conjugates as well as compositions, including compositions comprising antibody-therapeutic conjugates described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D. Pkd2 deletion at postnatal day 2 (P2) induced rapid cyst growth. Control mouse genotypes were Pkd2^{flox/+}:Cagg-Cre^{ER} or Pkd2^{null/flox}. Experimental mice had the genotype Pkd2^{null/flox}:Cagg-Cre^{ER}.

FIG. 1A shows the experimental timeline. Tamoxifen induction occurred at P2 followed by kidney collection for RNA sequencing at postnatal day 6 (P6) and postnatal day 10 (P10). Parallel mRNA and small RNA libraries were prepared.

FIG. 1B shows hematoxylin and eosin (H&E) staining of extensively disrupted organ architecture in experimental mice. Images were captured by Zeiss Axio Scan.Z1 with a 20× objective. The scale bar is 500 microns and applies to all images in the panel.

FIG. 1C is a graph of cystic burden in experimental (Exp) and control (Con) mice at postnatal days 6, 10, and 14. Cystic burden was quantified using the ratio of 2-kidney weight/body weight×100%. Sample numbers were: 28 (P6 Con), 27 (P6 Exp), 8 (P10 Con), 16 (P10 Exp), 6 (P14 Con), 9 (P14 Exp). Significance was shown by four asterisks (****) for p-values less than 0.0001 or “ns” for non-significant values as determined by one-way ANOVA followed by Tukey multiple comparison test with multiplicity-adjusted p-values. Error bars indicate standard deviation (SD).

FIG. 1D shows kidney sections probed for collecting duct marker Aquaporin-2 (red). Nuclei were marked by DAPI (4′,6-diamidino-2-phenylindole; blue). Images were captured by Zeiss Axio Scan.Z1 with a 20× objective. Enlargements (bottom row) from indicated regions (white outlined boxes) show details of collecting ducts at higher magnifying power. The scale bar is 500 microns (μm) for full size images and 100 microns (μm) for enlargements.

FIGS. 2A-2E. Cyst initiation candidate genes were identified by mRNA sequencing Pkd2 mouse kidneys at pre-cystic and early cystic time points.

FIG. 2A shows a volcano plot of pre-cystic postnatal day 6 (P6) differentially expressed genes (DEGs; analyzed using DESeq2) by log₂ of the fold-change (FC) in expression. Colored data points indicate a false discovery rate (FDR) of less than 0.05. Red data points indicate increased expression and blue data points indicate decreased expression in Experimental vs. Control mouse kidneys. The plot y-axis was cropped for better visualization. Asterisks (*) indicate data points that were moved for better visualization: Pkd2 (Log₂(FC)=−1.13, −Log₁₀(FDR)=93.97) and Cdkl1 (Log₂(FC)=0.42, −Log₁₀(FDR)=26.30).

FIG. 2B shows a volcano plot of early cystic postnatal day 10 (P10) DEGs (analyzed using DESeq2). Colored data points indicate FDR<0.05, red data points indicate increased expression, and blue data points indicate decreased expression in Experimental vs. Control mouse kidneys.

FIG. 2C is a heatmap showing the relative expression of all cyst initiation candidates (CICs) at P6 (left) and P10 (right).

FIG. 2D shows the intersection between 91 DEGs identified at P6 and 5,309 DEGs identified at P10. The intersection includes 74 genes which were defined as CICs.

FIG. 2E is a scatter plot showing correlation of CICs with published data sets. P6 DEG expression (Log₂ FC) is represented along the x-axis; DEG expression from P10 and published datasets are represented along the y-axis (Log₂ FC). Datasets include ZS (Pkd2) (23) and KP2, KP3, and KP6 (Pkd1; 2 weeks, 3 weeks, and 6 weeks post-induction, respectively) (22). Details of each study can be found in Table 8. There is significant correlation between P6 and each group by simple linear regression analysis (two-tailed p-values: P10, p<0.0001; ZS, p<0.0001; KP2, p=0.0014; KP3, p=0.0016; KP6, p=0.0012). Asterisks in the plot legend indicate significant p-values as follows: “****” indicates p<0.0001; “**” indicates p<0.01.

FIGS. 3A-3D. Differentially expressed mRNAs in pre-cystic and early cystic mouse kidneys.

FIG. 3A shows a functional enrichment analysis for P6 unique DEGs, P10 unique DEGs, and those common to both lists (CICs). Gene Ontology (GO), Kyoto Encyclopedia of Genes and Genomes (KEGG), Reactome, and WikiPathways databases were probed using g:profiler. Top functions are shown and listed, ranked by p adjusted values. See Tables 4-6.

FIG. 3B shows KEGG pathways found to be enriched for P10 DEGs.

FIG. 3C is a heatmap comparing differential gene expression for CICs at P6 and P10, and published pre-cystic mouse data from Woo et al. (14) (WP_Pkd2_P7, WP_Pkd2_P73, WP_Pkd2_P1, WP_Pkd1_P7, WP_Pkd1_P3, WP_Pkd1_P1), Zhang et al. (23) (ZS), and Kunnen et al. (22) (KP2, KP3, KP6). Tacstd2 is emphasized with a black box.

FIG. 3D is a series of bar plots showing the number of differentially expressed genes (false discovery rate <0.05) identified in data from the described studies. The top plot depicts data from this study (P6 and P10 following Pkd2 deletion). The center plot depicts data from Woo et al. (postnatal day 1 (P1), postnatal day 3 (P3), and postnatal day 7 (P7) following Pkd1 or Pkd2 deletion). The bottom plot depicts data from Kunnen et al. (weeks 2, 3, and 6 following Pkd1 deletion). Pkd1 models are represented by blue bars. Pkd2 models are represented by black bars.

FIG. 4. Small RNAseq of Pkd2 experimental vs. control mouse kidneys at postnatal day 10 (P10) identified microRNAs with known association in polycystic kidney disease (PKD). A volcano plot depicts P10 differentially expressed microRNAs (analyzed using omiRas pipeline). Colored data points indicate a false discovery rate (FDR) of less than 0.05. Red data points indicate upregulated expression and blue data points indicate downregulated expression in experimental vs. control mouse kidneys. The y-axis of the plot was cropped for better visualization of the data. MicroRNAs mir-133a-1, mir-133a-2, and mir-133a-3p were indistinguishable by small RNAseq and are therefore shown as one data point.

FIGS. 5A-5H. Single cell RNAseq repositories were utilized to find candidate genes expressed in tubule epithelial cells.

FIG. 5A is a Uniform Manifold Approximation and Projection (UMAP) visualization of murine kidney cell transcriptomic profiles and cluster identities from the kidney cell atlas by Ransick et al. (16). Clusters of cells were labeled as follows: Distal Convoluted Tubule (DCT), Proximal Tubule (PT), Intercalated Cell-A (IC-A), Intercalated Cell-B (IC-B), Principal Cell (PC), Inner Medullary Collecting Duct (IMCD), Descending Thin Limb of Henle (DTL), Ascending Thin Limb of Henle (ATL), and Medullary Thick Ascending Limb of Henle/Cortical Thick Ascending Limb of Henle (MTAL/CTAL).

FIG. 5B is a feature plot of the data presented in FIG. 5A highlighting the expression profile for Tacstd2 in healthy mouse kidney.

FIG. 5C is a DotPlot visualizing the localization of cyst initiation candidates (CICs) to the murine kidney cell atlas. Dot radius represents the percentage of cells in a cluster (x-axis) expressing a gene (y-axis). The opacity scale indicates average gene expression level within expressing cells. Each row was normalized. Main bars (far left) represent increased expression (red) or decreased expression (blue); sub bars (left) represent epithelial restricted expression (magenta), broad expression (green), and stromal/indeterminate expression (gray). A black arrow points to Tacstd2, an epithelial cyst initiating candidate gene. A blue arrow points to Pkd2.

FIG. 5D is a UMAP visualization of transcriptomic profiles of human kidney cells from healthy and autosomal dominant polycystic kidney disease (ADPKD) tissue; original data was from Muto et al. (19) and labeled by cluster identities set forth in Chen et al. (17). Clusters of cells were labeled as follows: Distal Convoluted Tubule (DCT), Proximal Tubule (PT), Intercalated Cell-A (IC-A), Intercalated Cell-B (IC-B), Principal Cell (PC), Inner Medullary Collecting Duct (IMCD), Descending Thin Limb of Henle (DTL), Ascending Thin Limb of Henle (ATL), and Medullary Thick Ascending Limb of Henle/Cortical Thick Ascending Limb of Henle (MTAL/CTAL).

FIG. 5E is a UMAP visualization of human kidney cells from healthy and ADPKD tissue (19). Clusters of cells were labeled as follows: Distal Convoluted Tubule (DCT), Proximal Tubule (PT), Intercalated Cell-A (IC-A), Intercalated Cell-B (IC-B), Principal Cell (PC), Inner Medullary Collecting Duct (IMCD), Thick Ascending Limb of Henle (TAL).

FIG. 5F is a DotPlot comparing TACSTD2 expression in healthy and ADPKD human kidneys in data from Muto et al. (19).

FIG. 5G is a pseudobulk heatmap showing expression of select epithelial candidate genes (based on murine data in FIG. 5B) in healthy vs. ADPKD human kidney tissue in data from Muto et al. (19). Main bars (far left) represent increased expression (red) or decreased expression (blue); sub bar (left) represent epithelial restricted expression (magenta). A black arrow points to Tacstd2.

FIG. 5H is a pseudobulk heatmap showing expression of candidate genes (based on murine data in FIGS. 2A-2E) in healthy (n=5) vs ADPKD (n=8) human kidney tissue (19). The candidates shown are differentially expressed using the Wilcox sum in “FindMarkers” and ordered by log 2FC. FDR<0.01. The red bar signifies increased expression in human ADPKD kidney (up in PKD) vs healthy control kidney. The blue bar signifies decreased expression in human ADPKD (down in PKD) vs healthy control kidney. Row-normalized expression.

FIGS. 6A-6E. Tacstd2 expression identified in cystic epithelium.

FIG. 6A is a DotPlot of marker genes according to Chen et al. (17) supporting the cell type assignments in FIG. 5A. Dot radius represents the percentage of cells in a cluster (x-axis) expressing a gene (y-axis). The opacity scale indicates average gene expression level within expressing cells. Each row was normalized. The first cell type shown on the x-axis is Renal Corpuscle/Podocyte.

FIG. 6B is a UMAP visualization from FIG. 5A with cell type assignments made according to Ransick et al. (16).

FIG. 6C is a DotPlot of the marker genes used to annotate FIG. 6B. Dot radius represents the percentage of cells in a cluster (x-axis) expressing a gene (y-axis). The opacity scale indicates average gene expression level within expressing cells. Each row was normalized.

FIG. 6D is a DotPlot of CICs as in FIG. 5C, but with cell type assignments from Ransick et al. (16). Dot radius represents the percentage of cells in a cluster (x-axis) expressing a gene (y-axis). The opacity scale indicates average gene expression level within expressing cells. Each row was normalized.

FIG. 6E shows two feature plots comparing TACSTD2 expression in healthy (left plot) and ADPKD (right plot) human kidney data from Muto et al. (19).

FIG. 6F shows two feature plots highlighting the diffusely increased expression profile for TACSTD2 in ADPKD (PKD; right plot) vs. healthy human kidneys (left plot).

FIGS. 7A-7C. Tacstd2 expression validated in mouse kidney epithelium.

FIG. 7A shows mouse kidney sections probed for Tacstd2 (shown in fuchsia in top row or white in bottom row) and DNA (DAPI, blue). Slide scans were captured by Zeiss Axio Scan.Z1 with a 20× objective. Scale bars are 500 microns (μm).

FIG. 7B shows quantification of the mean intensity of Tacstd2 fluorescence for control (Con) and experimental (Exp) mouse kidneys from three age groups: P6, P10, and postnatal day 21 (P21). Tacstd2 signal was normalized by the mean intensity of DAPI. Each experimental and age-matched control group had a sample size of n=3, except P6 experimental mice, which had a sample size of n=5. Mixed effects analysis with multiple comparisons was performed. Factors included age (p=0.0019) and genotype (p<0.0001). Asterisks in on the plot indicate significant p-values as follows: “****” indicates p<0.0001; “*” indicates p<0.05, and “ns” indicates a non-significant p-value.

FIG. 7C shows tissues described in FIG. 7A and FIG. 7B imaged using AiryScan LSM 900 with a 63× objective. Tissue sections were probed for Tacstd2 (white), collecting duct marker Aquaporin-2 (Aqp2, red), and proximal tubule marker Lotus tetragonolobus agglutinin (LTA, green). Nuclei were marked by DAPI (blue). Maximum intensity projections of 5-micron (μm) sections were imaged at 0.3 micron intervals. Scale bars are 10 microns.

FIG. 7D shows tissue that was stained for the collecting duct marker aquaporin2 (Aqp2, red), proximal tubule marker Lotus tetragonolobus Agglutinin (LTA, green), nuclei (DAPI, blue) and Tacstd2 (white). Tissues scans were captured by Zeiss Axio Scan.Z1 with 20× objective. Scale bars are 50 microns.

FIG. 8. Tacstd2 expression identified in cystic mouse renal epithelium. Images shown are all the scans used for quantification described in FIG. 7B. Scale bars are 1 mm.

FIGS. 9A-9B. TACSTD2 expression validated in human kidney epithelium.

FIG. 9A shows healthy and ADPKD human kidney sections probed for TACSTD2 (white), collecting duct marker Aquaporin-2 (AQP2, red), and proximal tubule marker Lotus tetragonolobus agglutinin (LTA, green). Nuclei were marked by DAPI (blue). Images were captured by Zeiss AxioScan.Z1 with a 20× objective. Scale bars are 100 microns (μm).

FIG. 9B shows mean intensity of TACSTD2 fluorescence as measured for normal healthy kidney (NHK) and ADPKD human kidney samples normalized by cellular area. Samples sizes were n=3 for both NHK and ADPKD. An unpaired t-test was performed. An asterisk (*) on the plot indicates a significant p-value of p=0.045.

FIGS. 10A-10G. TACSTD2 expression validated in human kidney organoids.

FIG. 10A shows a differentiation and maturation protocol for generating normal healthy kidney (NHK; control) and ADPKD (PKD1^{-/-} and PKD2^{-/-}) organoids from human induced pluripotent stem (IPS) cells.

FIG. 10B is a brightfield image of an organoid derived from control IPS cells. The protocol in FIG. 10A generates kidney organoids that gives rise to a majority of nephron components, excluding the most distal region, i.e., the collecting duct. Control human IPS cells differentiate into nephron progenitor cells within about one week. Nephron progenitor cells mature into renal epithelium within about 5 additional days. Organoids form within about one additional week. Normal healthy kidney organoids may be cultured for about 6 months.

FIG. 10C shows immunofluorescent staining of control organoids for markers of nephron components, which verified the presence of major renal structures. Renal proximal tubule brush borders are marked by Lotus tetragonolobus lectin (LTL, green) and phalloidin, which selectively labels F-actin present on the apical surface brush border of renal proximal tubules (red, second row only). Renal distal tubules are marked by high E-cadherin expression (fuchsia, fourth row only). The basement membrane on the basolateral surface of the renal epithelium lining kidney tubule is marked by laminin glycoprotein (red; first, third, and fourth rows). Primary cilia, which are present on the apical surface in vivo, are marked by enrichment of Arl13b GTPase (fuchsia, first through third rows).

FIG. 10D shows brightfield images of organoids derived from control (left), PKD1^{-/-} (center), and PKD2^{-/-} (right) human IPS cells. The scale bar is 200 microns (μm).

FIG. 10E shows cyst proliferation shown as cyst area as a percentage of total organoid area (%).

FIG. 10F shows immunofluorescence (IF) images of organoid sections stained for TACSTD2 (fuchsia in merged images on left; white in images in center), laminin (green) and DAPI (blue). The bottom row of images features the wall of a large cyst in a PKD1^{-/-} organoid; the IF images probe the area marked by a red box in brightfield image on the right. The scale bar is 20 microns (μm).

FIG. 10G shows quantification of the images shown in FIG. 10F. Statistical significance was determined by Welch and Brown-Forsythe ANOVA. Asterisks in the plot indicate p-values as follows: “****” indicates p<0.001; “***” indicates p<0.001; “**” indicates p<0.01, “*” indicates p<0.05, and “ns” means not significant.

FIGS. 11A-11F. Anti-TACSTD2 antibody-drug conjugate sacituzumab govitecan reduces the cyst area of high-TACSTD2-expressing PKD1^{-/-} and PKD2^{-/-} kidney organoids.

FIG. 11A shows control and PKD1^{-/-} kidney organoids treated with 0 nM (No SG) or 10 nM of the anti-TACSTD2 antibody-drug conjugate sacituzumab govitecan (SG) for a period of 30 days.

FIG. 11B shows quantification of change in organoid size as a percent of total organoid area at day 0 for wild-type control (WT), PKD1^{-/-}, and PKD2^{-/-} kidney organoids. PKD1^{-/-}, and PKD2^{-/-} organoids exhibited a decrease in size compared to wild-type control (WT) organoids when treated with sacituzumab govitecan.

FIG. 11C shows quantification of wild-type control (WT), PKD1^{-/-}, and PKD2^{-/-} normalized kidney organoid size over time without sacituzumab govitecan treatment.

FIG. 11D shows the data from FIG. 11C for wild-type control (WT) and PKD1^{-/-} kidney organoids. There is a significant difference between wild-type control (WT) and PKD1^{-/-} kidney organoid size after 11 days in culture. Insets show brightfield images of a wild-type control (WT) organoid and a PKD1^{-/-} organoid with significant cystic area. One asterisk (“*”) indicates a p-value of <0.05.

FIG. 11E shows quantification of wild-type control (WT), PKD1^{-/-}, and PKD2^{-/-} normalized kidney organoid size over time with 10 nM sacituzumab govitecan treatment. After 9 days of treatment with 10 nM sacituzumab govitecan (SG), there was a significant reduction in the size of PKD1^{-/-} organoids compared to wild-type control (WT) organoids. Two asterisks (“**”) indicate a p-value of <0.01.

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines and animals. The present disclosure is not limited by the illustrated ordering of acts or events, as some acts may occur in different orders and/or concurrently with other acts or events. Furthermore, not all illustrated acts, steps or events are required to implement a methodology in accordance with the present disclosure. Many of the techniques and procedures described, or referenced herein, are well understood and commonly employed using conventional methodology by those skilled in the art.

Headings used in this application are for convenience only and do not affect the interpretation of this application.

### Definitions

### General

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an” and “the” should be understood to include plural reference unless the context clearly indicates otherwise.

Throughout this specification and the claims which follow, unless the context requires otherwise, the word “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

“About” means within an acceptable error range for the particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of +20%, e.g., +10%, +5% or +1% of a given value. It is to be understood that the term “about” can precede any particular value specified herein, except for particular values used in the Exemplification. When “about” precedes a range, as in “about 24-96 hours,” the term “about” should be read as applying to both of the given values of the range, such that “about 24-96 hours” means about 24 hours to about 96 hours. As used herein, the symbol “+” denotes a range, that is, where a given value is “N±X” a range from N−X to N+X, wherein the range includes the values of N−X and N+X.

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the invention, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

When introducing elements disclosed herein, the articles “a,” “an,” “the,” and “said” are intended to mean that there are one or more of the elements. Further, the one or more elements may be the same or different. Thus, for example, unless the context clearly indicates otherwise, “an agent” includes a single agent, and two or more agents. Further the two or more agents can be the same or different as, for example, in embodiments wherein a first agent comprises a polynucleotide of a first sequence and a second agent comprises a polynucleotide of a second sequence.

As used herein, “increasing” refers to increasing by at least 5%, for example, 5, 6, 7, 8, 9, 10, 15, 20, 25, 30, 35, 40, 45, 50, 55, 60, 65, 70, 75, 80, 85, 90, 95, 99 or 100%, for example, as compared to the level of reference.

As used herein, “increases” also means increases by at least 1-fold, for example, 1-, 2-, 3-, 4-, 5-, 6-, 7-, 8-, 9-, 10-, 15-, 20-, 30-, 40-, 50-, 60-, 70-, 80-, 90-, 100-, 200-, 500-, or 1000-fold or more, for example, as compared to the level of a as compared to the level of a reference standard.

As used herein, “decreasing” refers to decreasing by at least 5%, for example, 5, 6, 7, 8, 9, 10, 15, 20, 25, 30, 35, 40, 45, 50, 55, 60, 65, 70, 75, 80, 85, 90, 95, 99, or 100%, for example, as compared to the level of reference.

As used herein, “decreases” also means decreases by at least 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of a reference.

As used herein, the term “reference” means a standard or control condition (e.g., untreated with a test agent or combination of test agents). Alternatively, “reference” may refer to a resource, such as an annotated genome, transcriptome, or the like, that is used to assemble, analyze, and/or interpret data.

As used herein, the term “eliminate” means to decrease to a level that is undetectable.

As used herein, the terms “treat,” “treating,” “treatment,” and the like refer to reducing or ameliorating a disease or disorder and/or symptoms associated therewith. For example, treatment can include a postponement or reduction in rate of development of the symptoms associated with a disease or disorder, and/or a reduction in the severity of such symptoms that will, or are expected, to develop with said disease. The terms include ameliorating existing symptoms, preventing additional symptoms, and ameliorating the underlying causes of such symptoms. Thus, the terms denote that a beneficial result is being conferred on at least some of the mammals, e.g., human patients, being treated. Many medical treatments are effective for some, but not all, patients that undergo the treatment. It will be appreciated that, although not precluded, treating a disorder or condition does not require that the disorder, condition or symptoms associated therewith be completely eliminated.

As used herein, the term “isolated” refers to a material that is free to varying degrees from components which normally accompany it as found in its native state. “Isolate” denotes a degree of separation from original source or surroundings.

### Subjects

The terms “subject” and “patient” are used interchangeably herein. The term “patient” refers to a human, while the term “subject” may refer to a human or a non-human animal.

As used herein, a “subject” is a vertebrate, including any member of the class mammalia.

As used herein, a “mammal” refers to any mammal including but not limited to human, mouse, rat, sheep, monkey, goat, rabbit, hamster, horse, cow or pig.

A “non-human mammal”, as used herein, refers to any mammal that is not a human.

In some embodiments, the subject is a human.

In some embodiments, the subject has one X chromosome and one Y chromosome. In some embodiments, the subject has two X chromosomes. In some embodiments, the subject has two X chromosomes and one Y chromosome. In some embodiments, the subject has one X chromosome and two Y chromosomes. In some embodiments, the subject is a human male. In some embodiments the subject is human female.

In some embodiments, the subject is at least about 1 month of age, for example, at least about 2, 3, 4, 5, 6, 7, 8, 9, 10, 11, 12, 15, 18 or 21 months of age, or at least about: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 years of age. In some embodiments, the subject is about: 1-100, 1-80, 1-60, 1-30, 1-24, 1-20, 1-18, 1-12, 1-10, 1-8, 1-6, 2-100, 2-80, 2-60, 2-30, 2-24, 2-20, 2-18, 2-12, 2-10, 2-8, 2-6, 3-100, 3-80, 3-60, 3-30, 3-24, 3-20, 3-18, 3-12, 3-10, 3-8, 3-6, 4-100, 4-80, 4-60, 4-30, 4-24, 4-20, 4-18, 4-12, 4-10, 4-8, 4-6, 5-100, 5-80, 5-60, 5-30, 5-24, 5-20, 5-18, 5-12, 5-10, 5-8, 6-100, 6-80, 6-60, 6-30, 6-24, 6-20, 6-18, 6-12, 6-10, 8-100, 8-80, 8-60, 8-30, 8-24, 8-20, 8-18, 8-12, 10-100, 10-80, 10-60, 10-30, 10-24, 10-20, 10-18, 12-100, 12-80, 12-38, 12-60, 12-50, 12-40, 12-30, 12-24, 12-20, 12-18, 18-100, 18-80, 18-60, 18-50, 18-40, 18-30, 18-24, 20-100, 20-80, 20-60, 20-50, 20-40, 20-30, 20-25, 30-100, 30-80, 30-60, 30-55, 30-50, 30-45, 30-40, 40-100, 40-80, 40-60, 40-55 or 40-50 years of age. In some embodiments, the subject is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80 or 100 years of age. In some embodiments, the subject is about 12-38 years of age. In other embodiments, the subject is a fetus. In some embodiments, the subject is a neonatal subject.

In some embodiments, the subject is 18 years of age or older, e.g., 18 to less than 40 years of age, 18 to less than 45 years of age, 18 to less than 50 years of age, 18 to less than 55 years of age, 18 to less than 60 years of age, 18 to less than 65 years of age, 18 to less than 70 years of age, 18 to less than 75 years of age, 40 to less than 75 years of age, 45 to less than 75 years of age, 50 to less than 75 years of age, 55 to less than 75 years of age, 60 to less than 75 years of age, 65 to less than 75 years of age, 60 to less than 75 years of age, 40 years of age or older, 45 years of age or older, 50 years of age or older, 55 years of age or older, 60 years of age or older, 65 years of age or older, 70 years of age or older, 75 years of age or older or 90 years of age or older. In some embodiments, the subject is 50 years of age or older. In some embodiments, the subject is a child. In some embodiments, the subject is 18 years of age or younger, e.g., 0-18 years of age, 0-12 years of age, 0-16 years of age, 0-17 years of age, 2-12 years of age, 2-16 years of age, 2-17 years of age, 2-18 years of age, 3-12 years of age, 3-16 years of age, 3-17 years of age, 3-18 years of age, 4-12 years of age, 4-16 years of age, 4-17 years of age, 4-18 years of age, 6-12 years of age, 6-16 years of age, 6-17 years of age, 6-18 years of age, 9-12 years of age, 9-16 years of age, 9-17 years of age, 9-18 years of age, 12-16 years of age, 12-17 years of age or 12-18 years of age.

In some embodiments, the subject is about 2-11, 4-17, 12-18, 18-50, 18-90 or 50-90 years of age.

### Compositions

In some embodiments described herein, a composition (e.g., a pharmaceutical composition) is administered to the subject. The phrase “pharmaceutically acceptable” means that the substance or composition the phrase modifies is, within the scope of sound medical judgment, suitable for use in contact with the tissues of humans and lower animals without undue toxicity, irritation, allergic response and the like, and are commensurate with a reasonable benefit/risk ratio.

As used herein, the term “pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds described herein include salts derived from suitable inorganic and organic acids, and suitable inorganic and organic bases.

Examples of salts derived from suitable acids include salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable salts derived from suitable acids include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

“Therapeutically effective amount” refers to an amount effective, at dosages and for periods of time necessary, to achieve the desired therapeutic result. A therapeutically effective amount may vary according to factors such as the disease state, age, sex, and weight of the individual, and the ability of a therapeutic or a combination of therapeutics to elicit a desired response in the individual.

In some embodiments, an agent disclosed herein (e.g., sacituzumab govitecan) is in a form of a pharmaceutical composition, or a pharmaceutically acceptable salt thereof. A “pharmaceutical composition” refers to a formulation of one or more therapeutic agents and a medium generally accepted in the art for delivery of a biologically active agent to subjects, e.g., humans. In some embodiments, a pharmaceutical composition may include one or more pharmaceutically acceptable excipients, diluents, or carriers. “Pharmaceutically acceptable carrier, diluent, or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In some embodiments, a pharmaceutical composition disclosed herein is formulated as a solution.

“Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative. In some embodiments, the carrier may be a diluent, adjuvant, excipient, or vehicle with which the agent (e.g., polynucleotide) is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the agent in such pharmaceutical formulation may vary widely, i.e., from less than about 0.5%, to at least about 1%, or to as much as 15% or 20%, 25%, 30%, 35%, 40%, 45% or 50% by weight. The concentration will be selected primarily based on required dose, fluid volumes, viscosities, etc., according to the mode of administration. Suitable vehicles and formulations, inclusive of other human proteins, e.g., human serum albumin, are described, for example, in Remington: The Science and Practice of Pharmacy, 21^{st} Edition, Troy, D. B. ed., Lipincott Williams and Wilkins, Philadelphia, PA 2006, Part 5, Pharmaceutical Manufacturing: 691-1092 (e.g., pages 958-89).

In some embodiments, a pharmaceutical composition suitable for use in methods disclosed herein further comprises one or more pharmaceutically acceptable carriers. The term “pharmaceutically acceptable carrier” refers to an ingredient in a pharmaceutical composition, other than an active ingredient, which is nontoxic to a subject and should not interfere with the efficacy of the active ingredient. A pharmaceutically acceptable carrier includes, but is not limited to, such as those widely employed in the art of drug manufacturing. The carrier may be a diluent, adjuvant, excipient, or vehicle with which the agent is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the agent in such pharmaceutical formulation may vary widely, e.g., from less than about 0.5%, usually to at least about 1% to as much as 15%, 20%, 25%, 30%, 35%, 40%, 45% or 50% by weight. The concentration will be selected primarily based on required dose, fluid volumes, viscosities, etc., according to the particular mode of administration selected. Suitable vehicles and formulations, inclusive of other human proteins, e.g., human serum albumin, are described, for example, in e.g., Remington: The Science and Practice of Pharmacy, 21^{st} Edition, Troy, D. B. ed., Lipincott Williams and Wilkins, Philadelphia, Pa. 2006, Part 5, Pharmaceutical Manufacturing pp 691-1092, see especially pp. 958-89.

Non-limiting examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

Non-limiting examples of buffers that may be used are acetic acid, citric acid, formic acid, succinic acid, phosphoric acid, carbonic acid, malic acid, aspartic acid, histidine, boric acid, Tris buffers, HEPPSO and HEPES.

Non-limiting examples of antioxidants that may be used are ascorbic acid, methionine, cysteine hydrochloride, sodium bisulfate, sodium metabisulfite, sodium sulfite, lecithin, citric acid, ethylenediamine tetraacetic acid (EDTA), sorbitol and tartaric acid.

Non-limiting examples of amino acids that may be used are histidine, isoleucine, methionine, glycine, arginine, lysine, L-leucine, tri-leucine, alanine, glutamic acid, L-threonine, and 2-phenylamine.

Non-limiting examples of surfactants that may be used are polysorbates (e.g., polysorbate-20 or polysorbate-80); polyoxamers (e.g., poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g., PLURONICS™, PF68, etc.).

Non-limiting examples of preservatives that may be used are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl and the like), benzalkonium chloride, benzethonium chloride, sodium dehydroacetate and thimerosal, or mixtures thereof.

Non-limiting examples of saccharides that may be used are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, melezitose, raffinose, mannotriose, stachyose, maltose, lactulose, maltulose, glucitol, maltitol, lactitol or iso-maltulose.

Non-limiting examples of salts that may be used are acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. In some embodiments, the salt is sodium chloride (NaCl).

Agents (e.g., sacituzumab govitecan) disclosed herein may be prepared in accordance with standard procedures and are administered at dosages that are selected to reduce, prevent, or eliminate, or to slow or halt progression of, a condition being treated (See, e.g., Remington's Pharmaceutical Sciences, Mack Publishing Company, Easton, PA, and Goodman and Gilman's The Pharmaceutical Basis of Therapeutics, McGraw-Hill, New York, N.Y., the contents of which are incorporated herein by reference, for a general description of methods for administering various agents for human therapy).

### Administration

In some embodiments, an agent disclosed herein (e.g., sacituzumab govitecan) is delivered using controlled or sustained-release delivery systems (e.g., capsules, biodegradable matrices). Example delayed-release delivery systems for drug delivery that would be suitable for administration of a composition described herein are described in U.S. Pat. No. 5,990,092 (issued to Walsh); U.S. Pat. No. 5,039,660 (issued to Leonard); U.S. Pat. No. 4,452,775 (issued to Kent); and U.S. Pat. No. 3,854,480 (issued to Zaffaroni), the entire teachings of which are incorporated herein by reference. Example biodegradable matrices are discussed in Li et al. (Li, C., Guo, C., Fitzpatrick, V. et al. Design of biodegradable, implantable devices towards clinical translation. Nat Rev Mater 5, 61-81 (2020). doi.org/10.1038/s41578-019-0150-z).

For oral administration, an agent such as sacituzumab govitecan may be in the form of, for example, a tablet, capsule, suspension or liquid. An agent such as sacituzumab govitecan is preferably made in the form of a dosage unit containing a therapeutically effective amount of an active ingredient. Examples of such dosage units are tablets and capsules. For therapeutic purposes, tablets and capsules can contain, in addition to an active ingredient, conventional carriers such as binding agents, for example, acacia gum, gelatin, polyvinylpyrrolidone, sorbitol, or tragacanth; fillers, for example, calcium phosphate, glycine, lactose, maize-starch, sorbitol, or sucrose; lubricants, for example, magnesium stearate, polyethylene glycol, silica, or talc; disintegrants, for example potato starch, flavoring or coloring agents, or acceptable wetting agents. Oral liquid preparations generally in the form of aqueous or oily solutions, suspensions, emulsions, syrups or elixirs may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

Administration of the agent (e.g., the antibody-therapeutic conjugate) to the subject can be by parenteral or non-parenteral means. In some embodiments, an agent disclosed herein (e.g., sacituzumab govitecan) is administered intravenously, intra-arterially, intrathecally, intraventricularly, intramuscularly, intradermally, subcutaneously, intracranially, intrarenally, or spinally. “Administering” or “administration” as used herein, refers to taking steps to deliver an agent to a subject, such as a mammal, in need thereof. Administering can be performed, for example, once, a plurality of times, and/or over one or more extended periods. Administration includes both direct administration, including self-administration, and indirect administration, including an act of prescribing a drug or directing a subject to consume an agent. For example, as used herein, one (e.g., a physician) who instructs a subject (e.g., a patient) to self-administer an agent (e.g., a drug), or to have an agent administered by another and/or who provides a patient with a prescription for a drug is administering an agent to a subject. Administration of an agent can be once in a day or more than once in a day (e.g., twice a day or more). Administration of the agent can be repeated after one day, two days, three days, four days, five days, six days, one week, two weeks, three weeks, four weeks, five weeks, six weeks, seven weeks, two months, three months, four months, five months, six months or longer. Repeated courses of treatment are also possible, as is chronic administration. The repeated administration may be at the same dose or at a different dose.

In some embodiments, an agent disclosed herein (e.g., sacituzumab govitecan) is delivered systemically, such as via intravenous or subcutaneous injection. In some embodiments, the agent (e.g., sacituzumab govitecan) is delivered using an approach that enhances bioavailability in the target tissue or organ after administration. These approaches can include modification of the sugars or phosphate linkages, delivering as a duplex with a ligand-conjugated RNA molecule, formulation into an artificial exosome, liposome, polymer nanoparticle or lipid nanoparticle, or conjugation to lipids, antibodies, peptides, sugars, neuroactive molecules, or other moieties that enhance delivery to the target tissue or organ. Other methods of enhancing bioavailability in the target tissue or organ after administration will be known to one skilled in the art.

In some embodiments, a method disclosed herein comprises administering to the subject two or more agents, for example, 2, 3, 4, or 5 or more agents. In some embodiments, the two or more agents are administered together. In other embodiments, the two or more agents are administered separately, e.g., sequentially. In some embodiments, two or more agents are administered in the same composition. In some embodiments, two or more agents are administered in different compositions.

In some embodiments, a composition for administration comprises an antibody-drug conjugate (ADC) and an additional therapeutic agent. In some embodiments, the ADC comprises sacituzumab govitecan. In some embodiments, a composition for administration comprises sacituzumab govitecan and, for example, a vasopressin receptor antagonist (e.g., tolvaptan), an angiotensin-converting enzyme (ACE) inhibitor (e.g., lisinopril), or an angiotensin receptor blocker (ARB; e.g., losartan), a sclerosing agent (e.g., ethanol or sodium tetradecyl sulfate), an analgesic that is not a nonsteroidal anti-inflammatory drug (NSAID; e.g., acetaminophen), or the like, or a combination thereof. In some embodiments, an ADC is administered in combination with at least one additional therapeutic agent.

Agents such as antibody-drug conjugates and their dosages, routes of administration and recommended treatment protocols are known in the art and have been described in such literature as the Physician's Desk Reference (56th ed., 2002 and 57th ed., 2003). Generally, a therapeutically effective amount of an antibody or conjugate described herein is in the range of 0.1 mg/kg to 100 mg/kg, preferably 0.1 mg/kg to 50 mg/kg. In some embodiments, a therapeutically effective amount of an antibody or conjugate described herein is about 10 mg/kg. In some embodiments, a therapeutically effective amount of an antibody or conjugate described herein is about 10 mg/kg administered on days 1 and 8 of a 3-week cycle for up to 8 cycles, or until disease progression or unacceptable toxicity contraindicate continued administration.

### Functional Agents (e.g., Antibodies, Inhibitors, Etc.)

As used herein, an “antibody” is any immunoglobulin polypeptide, or fragment thereof, having immunogen binding ability. As used herein, the term “anti-,” when used with respect to an antibody, precedes the name of a target to which an antibody binds.

In some embodiments, an “antibody” refers to a whole (intact) antibody, or an antigen-binding fragment of an antibody. As used herein, the term “antibody” refers to an immunoglobulin molecule capable of specific binding to a target, such as a carbohydrate, polynucleotide, lipid, polypeptide, etc., through at least one antigen recognition site, located in the variable region of the immunoglobulin molecule. The antibody can be of any species, such as a murine antibody, a human antibody or a humanized antibody. In some embodiments, the antibody is a chimeric antibody. In some embodiments, the term “antibody” refers to a full-length antibody comprising two heavy (H) chains and two light (L) chains inter-connected by disulfide bonds or multimers thereof (for example, IgM). Each heavy chain comprises a heavy chain variable region (V_{H}) and a heavy chain constant region. Each light chain comprises a light chain variable region (V_{L}) and a light chain constant region.

The term “antigen-binding fragment” refers to a portion of an immunoglobulin molecule (e.g., an antibody) that retains the antigen binding properties of the parental full-length antibody. As used herein, the term “antibody” may refer to an antigen-binding fragment (ABF). Non-limiting examples of antigen-binding fragments include a V_{H} region, a V_{L} region, an Fab fragment, an F(ab′)₂ fragment, an Fd fragment, an Fv fragment, and a domain antibody (dAb) consisting of one V_{H} domain or one V_{L} domain, etc. V_{H} and V_{L} domains may be linked together via a synthetic linker to form various types of single-chain antibody designs in which the V_{H}/V_{L} domains pair intramolecularly, or intermolecularly in those cases when the V_{H} and V_{L} domains are expressed by separate chains, to form a monovalent antigen binding site, such as single chain Fv (scFv) or diabody.

The antibodies described herein can be monospecific, or multispecific (e.g., bispecific or trispecific). The term “bispecific antibody” refers to an antibody capable of binding two distinct antigens simultaneously via two distinct antigen binding sites. A “trispecific antibody” refers to an antibody capable of binding three distinct antigens simultaneously via three distinct antigen binding sites. A “multispecific antibody” refers to an antibody capable of binding multiple distinct antigens simultaneously via multiple distinct antigen binding sites.

As used herein, a “fragment” is a portion of a polypeptide or nucleic acid molecule. This portion contains in example embodiments, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

As used herein, an “agonist” is an agent that causes an increase in the expression or activity of a target gene or protein, respectively. An agonist can bind to and activate its cognate receptor in some fashion, which directly or indirectly brings about this physiological effect on the target gene or protein.

As used herein, an “inhibitor” is an agent that causes a decrease in the expression or activity of a target gene or protein, respectively. An “antagonist” can be an inhibitor but is more specifically an agent that binds to a receptor, which in turn decreases or eliminates binding by other molecules.

As used herein, “antibody-therapeutic conjugate” or “ATC” refers to an antibody covalently bonded to a therapeutic agent. In some embodiments, an ATC is an “antibody-drug conjugate.” As used herein, “antibody-drug conjugate” or “ADC” refers to an antibody covalently bonded to a therapeutic drug, such as a small molecule (e.g. SN-38).

### Genes, Transcripts, Proteins, and Omics

In general, as used herein, italicized symbols indicate genes or transcripts and non-italicized symbols indicate proteins. In general, as used herein, all-capital symbols indicate human origin; and symbols with only the first letter capitalized indicate mouse or other animal origin. As used herein, gene, transcript, and protein symbols may utilize lowercase and uppercase letters interchangeably where species information is provided by context, or where the referent gene, transcript, or protein is analogous across multiple species. As used herein, gene and transcript symbols may be italicized or non-italicized, with information on the type of nucleic acid provided by context.

As used herein, the term “functionally express” refers to the ability of a cell to express an RNA and/or protein at a level, or within a range, that is known in the art to correlate with an identity and/or function of a cell. In some embodiments, functional expression is of a transcript and/or a protein. In some embodiments, functional expression, or the lack thereof, is detected by quantitative polymerase chain reaction (qPCR), RNA sequencing (RNA-seq), flow cytometry, mass cytometry, immunofluorescence (IF), immunohistochemistry (IHC), immunocytochemistry (ICC), in situ hybridization (ISH), fluorescent in situ hybridization (FISH), or other methods or techniques capable of detecting an RNA, a protein, or a portion thereof. In some embodiments, detection of RNA is an acceptable surrogate for protein detection where protein detection has been validated and/or a reasonable expectation of successful translation exists.

As used herein, the human protein “TACSTD2” (e.g., UniProtKB Accession P09758) and its murine homolog “Tacstd2” (e.g., UniProtKB Accession Q8BGV3) refer to the tumor-associated calcium signal transducer 2 protein. TACSTD2 is also known as trophoblast cell surface antigen 2 (TROP2 or TROP-2), epithelial glycoprotein-1 (EGP1 or EGP-1), membrane component chromosome 1 surface marker 1 (M1S1), GA733-1, and other aliases. As used herein, the term “differentially expressed” refers to a gene, RNA, or protein that is expressed at different levels in two or more groups. A group may be, for example, a cell, a population of cells, or a tissue, and/or a pool of cells or tissues from more than one organism. As used herein, a group reflects an experimental condition, for example, a particular genotype or treatment with a drug. As used herein, the term “differentially expressed gene” refers a gene that is differentially expressed or to the transcriptional product (e.g., mRNA) of a gene that is differentially expressed.

### Cells, Cell Culture, and Differentiation

As used herein, the term “stem cell” refers to a multipotent cell having the capacity to self-renew and to differentiate into multiple cell lineages.

As used herein, the term “epithelial stem cell” refers to a multipotent cell which has the potential to become committed to multiple cell lineages, including cell lineages resulting in epithelial cells.

As used herein, the term “progenitor cell” refers to a lineage-restricted cell derived from a stem cell.

As used herein, the term “epithelial progenitor cell” refers to a multipotent cell which has the potential to become restricted to cell lineages resulting in epithelial cells.

As used herein, the term “self-renewal” refers to the process by which a stem cell divides to generate one (asymmetric division) or two (symmetric division) daughter cells with development potentials that are indistinguishable from those of the mother cell. Self-renewal involves both proliferation and the maintenance of an undifferentiated state.

As used herein, a “population” of cells is any number of cells greater than 1, such as at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, the term “organoid” or “epithelial organoid” refers to a cell cluster or aggregate that resembles an organ, or part of an organ, and possesses cell types relevant to that particular organ. Alternatively, “organoid,” “3D culture,” “re-aggregate,” “reaggregated organ culture (ROC),” or “droplet organ culture” refers to a model of an organ or tissue, e.g., a simplified and/or miniaturized version of the organ or tissue that recapitulates characteristics of the organ or tissue in a state that is undeveloped, developing, fully developed, mature, aging, healthy, injured, diseased, and/or at risk for disease. An organoid can be a model of any stage organ development. The terms “organoid,” “3D culture,” “re-aggregate,” “reaggregated organ culture (ROC),” and “droplet organ culture” may be used interchangeably herein.

“Singularizing” refers to the process of dissociating cell aggregates, i.e., adherent cells (e.g., cells attached to other cells and/or cells attached to a growth surface or matrix), into a suspension comprising cells that are primarily singularized, i.e. not attached to other cells or not attached to many other cells. Singularizing may comprise dissociating cells with a proteolytic enzyme capable of detaching and/or dissociating adherent cells in culture, scraping the cells and then triturating the cells by pipette, or a combination thereof. The proteolytic enzyme, or a solution containing the proteolytic enzyme, may comprise any enzyme capable of dissociating adherent cells, non-limiting examples of which include trypsin (e.g., Cocoonase, Tryptar, Tryptase; CAS No. 9002-07-7), TRYPLE® recombinant dissociation enzyme, ACCUTASE® cell detachment solution, DISPASE® dissociating enzyme, or a combination thereof.

“Plating” refers to the action of dispensing cells (i.e., singularized cells or small aggregates of cells) onto/into a plate. As used herein, “seeding” refers to plating a given number of cells with the intention of culturing said cells to divide and produce a larger number of cells.

As used herein, “differentiating” refers to culturing a multipotent cell in such a way that its potential to become committed to multiple cell lineages is reduced, i.e., such that the multipotent cell exhibits, to varying degrees, a more differentiated cell identity. Culturing or differentiating cells may comprise culturing the cells on a basement membrane or extracellular matrix. Plates (e.g. bottoms of wells of tissue culture plates) may be coated with a basement membrane (BM) or extracellular matrix (ECM), of which CORNING® MATRIGEL® hESC-Qualified Matrix (Corning, Corning, NY; Cat. No. 354277) is a non-limiting example. “MATRIGEL®” refers to a basement membrane matrix produced by Engelbreth-Holm-Swarm mouse sarcomas and is commonly used to support the culture of multipotent cells, cells in the process of differentiation, and 3D/organoid cultures. The matrix may be growth factor reduced, for example, growth factor reduced MATRIGEL® (MATRIGEL® (GFR). MATRIGEL® (GFR) differs from regular MATRIGEL® by an additional treatment with ammonium sulfate, resulting in the reduction of growth factors bound to the extracellular matrix components contained within the MATRIGEL®. A different basement membrane or extracellular matrix may be used, non-limiting examples of which include: Matrigengel Matrix Growth Factor Reduced basement membrane extracts (ACRO Biosystems, Newark, DE; Cat. No. AC-M082701), CULTREX® Reduced Growth Factor Basement Membrane Extract (R&D Systems, Minneapolis, MN; Cat. No. 3433-005-01), Vitronectin XF™ defined xeno-free cell culture matrix (STEMCELL Technologies, Vancouver, Canada; Cat. No. 07180), GIBCO® GELTREX® LDEV-Free Reduced Growth Factor Basement Membrane Matrix (Thermo Fisher Scientific, Waltham, MA; Cat. No. A1413201), and the like.

The terms “media” and “medium” may be used interchangeably herein when in reference to cell culture. A medium may comprise one or more supplements, such as a small molecule, an amino acid, a protein, a serum, a nutrient mixture, and the like, or a combination thereof. In some embodiments, a disclosed supplement may be replaced by a “suitable substitute.” In some embodiments, a suitable substitute comprises a supplement or combination of supplements that is different from but equivalent to the disclosed supplement, i.e. a supplement that exhibits the same or a similar biological effect as the disclosed supplement.

In some embodiments, a medium is supplemented with a GSK inhibitor. In some embodiments, a GSK3 inhibitor is a WNT agonist, a GSK-3β inhibitor, or both. In some embodiments, a GSK inhibitor may inhibit additional pathways and/or proteins. WNT agonists comprising GSK-3β inhibitors are well known in the art and include, but are not limited to, 1,6-[[2-[[4-(2,4-Dichlorophenyl)-5-(5-methyl-1H-imidazol-2-yl)-2-pyrimidinyl]amino]ethyl]amino]-3-pyridinecarbonitrile “CHIR99021” (Ring et al., 2003), LiCl (Klein et al., 1996), BIO-acetoxime ((2′Z,3′E)-6-Bromoindirubin-3′-oxime) (Meijer et al., 2003), N6-[2-[[4-(2,4-Dichlorophenyl)-5-(1H-imidazol-2-yl)-2-pyrimidinyl]amino]ethyl]-3-nitro-2,6-pyridinediamine “CHIR98014” (Ring et al., 2003), 3-(2,4-Dichlorophenyl)-4-(1-methyl-1H-indol-3-yl)-1H-pyrrole-2,5-dione “SB 216763” also known as GSK-3 Inhibitor IV (Coghlan et al., 2000), 3-[(3-Chloro-4-hydroxyphenyl)amino]-4-(2-nitrophenyl)-1H-pyrrol-2,5-dione “SB 415286” (Coghlan et al., 2000), 5-ethyl-7,8-dimethoxy-1H-pyrrolo[3,4-c]-isoquinoline-1,3-(2H)-dione “3F8” (Zhong et al., 2009), 9-Bromo-7,12-dihydro-indolo[3,2-d][1]benzazepin-6(5H)-one “Kenpaullone” (Schultz et al., 1999; Zaharevitz et al., 1999), 9-Bromo-7,12-dihydro-pyrido[3′,2′:2,3]azepino[4,5-b]indol-6(5H)-one “1-Azakenpaullone” (Schultz et al., 1999; Zaharevitz et al., 1999), N-(3-Chloro-4-methylphenyl)-5-(4-nitrophenyl)-1,3,4-oxadiazol-2-amine “TC-G 24” (Khanfar et al., 2010), 2-Methyl-5-[3-[4-(methylsulfinyl)phenyl]-5-benzofuranyl]-1,3,4-oxadiazole “TCS 2002” (Saitoh et al., 2009), N-[(4-Methoxyphenyl)methyl]-N′-(5-nitro-2-thiazolyl) urea “AR-A 014418” (Bhat et al., 2003), 3-[5-[4-(2-Hydroxy-2-methyl-1-oxopropyl)-1-piperazinyl]-2-(trifluoromethyl)phenyl]-4-(1H-indol-3-yl)-1H-pyrrole-2,5-dione “TCS 21311” (Thoma et al., 2011), 3-[[6-(3-aminophenyl)-7H-pyrrolo[2,3-d]pyrimidin-4-yl]oxy]-phenol “TWS 119” (Ding et al., 2003), ((2′Z,3′E)-6-Bromoindirubin-3′-acetoxime) “BIO-acetoxime” also known as GSK-3 Inhibitor IX (Meijer et al., 2003), 4-(2-Amino-4-oxo-2-imidazolin-5-ylidene)-2-bromo-4,5,6,7-tetrahydropyrrolo[2,3-c]azepin-8-one “10Z-Hymenialdisine” (Breton et al., 1997), 2-[(3-iodophenyl)methylsulfanyl]-5-pyridin-4-yl-1,3,4-oxadiazole, also known as GSK-3ß Inhibitor II (Wada, 2009), 4-Benzyl-2-methyl-1,2,4-thiadiazolidine-3,5-dione, also known as GSK-3ß Inhibitor I (Wada, 2009), 3-Amino-6-(4-((4-methylpiperazin-1-yl) sulfonyl)phenyl)-N-(pyridin-3-yl) pyrazine-2-carboxamide, HCl, also known as GSK-3ß Inhibitor XXVII (US Patent Pub. No. 2006/0173014), 4,5-bis(1-Methyl-1H-indol-3-yl)-1,2-dihydropyrazol-3-one, also known as GSK-3ß Inhibitor XXVI (Chen et al, 2011), FRATtide peptide (Bax et al., 2001), 3-Amino-1H-pyrazolo[3,4-b]quinoxaline “Cdk1/5 Inhibitor” (Andreani et al., 1996, 2000; Katoh et al., 2011) and 4-((5-Bromo-2-pyridinyl)amino)-4-oxobutanoic acid “Bikinin” (De Rybel et al., 2009).

In some embodiments, a medium is supplemented with a ROCK (Rho kinase) inhibitor. In some embodiments, a ROCK inhibitor comprises a Rho kinase inhibitor, a Rho-associated protein kinase inhibitor, a ROCK1 inhibitor, a ROCK 2 inhibitor, or a combination thereof. In some embodiments, a ROCK inhibitor may additionally inhibit other pathways and/or proteins. In some embodiments, a ROCK inhibitor comprises Y-27632 (trans-4-[(1R)-1-Aminoethyl]-N-4-pyridinylcyclohexanecarboxamide, CAS No. 146986-50-7), Y-27632 2HCl (trans-4-[(1R)-1-Aminoethyl]-N-4-pyridinylcyclohexanecarboxamide dihydrochloride, y27632, Y-27632 2HCl, ROCK inhibitor, ROCK1 inhibitor, CAS No. 129830-38-2), GSK429286A (4-[4-(Trifluoromethyl)phenyl]-N-(6-Fluoro-1H-indazol-5-yl)-2-methyl-6-oxo-1,4,5,6-tetrahydro-3-pyridinecarboxamide, GSK 429286, CAS No. 864082-47-3), RKI-1447 (N-[(3-Hydroxyphenyl)methyl]-N′-[4-(4-pyridinyl)-2-thiazolyl]urea dihydrochloride, RKI 1447 dihydrochloride, CAS No. 1782109-09-4), Chroman 1 ((3S)-N-[2-[2-(Dimethylamino) ethoxy]-4-(1H-pyrazol-4-yl)phenyl]-3,4-dihydro-6-methoxy-2H-1-benzopyran-3-carboxamide dihydrochloride, CAS No. 1273579-40-0), a CEPT cocktail comprising Chroman 1, Emricasan (N-[2-(1,1-Dimethylethyl)phenyl]-2-oxoglycyl-N-[(1S)-1-(carboxymethyl)-2-oxo-3-(2,3,5,6-tetrafluorophenoxy) propyl]-L-alaninamide, CAS No. 254750-02-2), Putrescine (1,4-Butanediamine dihydrochloride, 1,4-Diaminobutane dihydrochloride, Tetramethylenediamine dihydrochloride, Putrescine dihydrochloride, CAS No. 333-93-7), Spermine (N,N′-Bis(3-aminopropyl)-1,4-butanediamine tetrahydrochloride, Spermine tetrahydrochloride, CAS No. 306-67-2), Spermidine (1,8-Diamino-4-azaoctane, N-(3-Aminopropyl)-1,4-diaminobutane, CAS No. 124-20-9), and trans-ISRIB (N,N′-trans-1,4-cyclohexanediylbis[2-(4-chlorophenoxy) acetamide], CAS No. 1597403-47-8); or the like, or a combination thereof.

### Model Organisms

As used herein, the terms “mouse strain” and “mouse line” refer to mice that are bred specifically to maintain a certain genotype, which may be wild-type or contain one or more mutations (e.g., substitution, insertion, deletion, early stop codon), chromosomal rearrangements (e.g., translocation, duplication, inversion), transgenes (i.e., a sequence from a different species of organism), knocked-in (i.e., inserted) sequences, knocked-out (i.e., deleted) sequences, floxed sequences (i.e., a sequence flanked by loxP sites), and the like.

Multiple mouse strains may be used to create the strains cited and used herein, or functional equivalents thereto. Non-limiting examples include: ROSAβ-geo26 (see Friedrich G, Soriano P. Promoter traps in embryonic stem cells: a genetic screen to identify and mutate developmental genes in mice. Genes Dev. 1991 September; 5 (9): 1513-23. Doi: 10.1101/gad.5.9.1513. PMID: 1653172.), the ROSA26 reporter strain R26R (see Soriano P. Generalized lacZ expression with the ROSA26 Cre reporter strain. Nat Genet. 1999 January; 21 (1): 70-1. doi: 10.1038/5007. PMID: 9916792.), the inducible Cre-ERT strain with a G521R mutation in ER (see Feil R, Brocard J, Mascrez B, LeMeur M, Metzger D, Chambon P. Ligand-activated site-specific recombination in mice. Proc Natl Acad Sci USA. 1996 Oct. 1; 93 (20): 10887-90. doi: 10.1073/pnas.93.20.10887. PMID: 8855277; PMCID: PMC38252.), the inducible Cre-ERT2 strain with G400V/M543A/L540A mutations in ER (see Indra A K, Warot X, Brocard J, Bornert J M, Xiao J H, Chambon P, Metzger D. Temporally-controlled site-specific mutagenesis in the basal layer of the epidermis: comparison of the recombinase activity of the tamoxifen-inducible Cre-ER(T) and Cre-ER(T2) recombinases. Nucleic Acids Res. 1999 Nov. 15; 27 (22): 4324-7. doi: 10.1093/nar/27.22.4324. PMID: 10536138; PMCID: PMC148712. See also Feil R, Wagner J, Metzger D, Chambon P. Regulation of Cre recombinase activity by mutated estrogen receptor ligand-binding domains. Biochem Biophys Res Commun. 1997 Aug. 28; 237 (3): 752-7. doi: 10.1006/bbrc.1997.7124. PMID: 9299439.), and the Rosa26-CreERT2 strain (see Ventura A, Kirsch D G, McLaughlin M E, Tuveson D A, Grimm J, Lintault L, Newman J, Reczek E E, Weissleder R, Jacks T. Restoration of p53 function leads to tumour regression in vivo. Nature. 2007 Feb. 8; 445(7128):661-5. doi: 10.1038/nature05541. Epub 2007 Jan. 24. PMID: 17251932.).

Other definitions appear in context throughout this disclosure.

### Example Embodiments

In one aspect, the disclosure herein provides a method of treating a subject in need thereof, comprising administering to the subject a therapeutically effective amount of an antibody-therapeutic conjugate, wherein the subject has or is predisposed to have a polycystic kidney disease (PKD). In some embodiments, the subject has PKD. In some embodiments, the subject is predisposed to have PKD. In some embodiments, a subject is predisposed to have PKD due to one or more variants (e.g., pathogenic variants, inactivating mutations) in genes encoding either of two transmembrane proteins, polycystin-1 (PKD1 in humans, Pkd1 in mice) or polycystin-2 (PKD2/Pkd2). In some embodiments, a subject is predisposed to have PKD due to variants (e.g., pathogenic variants, inactivating mutations) in PKHD1 ciliary IPT domain containing fibrocystin/polyductin (PKHD1/Pkhd1).

In some embodiments, the antibody-therapeutic conjugate comprises a whole antibody or an antigen-binding fragment thereof.

In some embodiments, the antibody-therapeutic conjugate comprises an anti-TACSTD2 antibody or antigen-binding fragment thereof.

In some embodiments, the antibody-therapeutic conjugate comprises an antibody selected from the group comprising sacituzumab, datopotamab, and RN926, or antigen-binding fragment thereof.

In some embodiments, the antibody-therapeutic conjugate comprises a monospecific, bispecific or multispecific antibody, e.g., a whole antibody or antigen-binding fragment thereof.

In some embodiments, the antibody-therapeutic conjugate comprises a bispecific antibody that binds TACSTD2 and an additional antigen.

In some embodiments, the methods described herein relate to administration of an antibody-therapeutic conjugate comprising a therapeutic agent.

In some embodiments, the antibody-therapeutic conjugate comprises a cytotoxic drug, a cytostatic drug, or a gene therapy, or a combination thereof.

In some embodiments, the gene therapy comprises gene editing, gene augmentation, gene replacement, or gene addition.

In some embodiments, the gene therapy targets PKD1 or PKD2.

In some embodiments, the gene therapy comprises an anti-sense oligonucleotide (ASO) or small interfering RNA (siRNA).

In some embodiments, the gene therapy targets TACSTD2.

In some embodiments, the gene therapy comprises a viral vector or a non-viral delivery system.

In some embodiments, the antibody-therapeutic conjugate comprises a topoisomerase I inhibitor, a tubulin polymerization inhibitor, a vasopressin receptor 2 antagonist, or an mTOR inhibitor, or a combination thereof.

In some embodiments, the antibody-therapeutic conjugate comprises one or more therapeutic agents selected from the group comprising SN38, DXd, Aur0101, tolvaptan, and rapamycin.

In some embodiments, the antibody-therapeutic conjugate comprises one or more of sacituzumab govitecan (hRS7-SN38), datopotamab deruxtecan (Dato-DXd), or PF-06664178 (RN926-Aur0101).

In some embodiments, the polycystic kidney disease is autosomal dominant polycystic kidney disease (ADPKD).

In some embodiments, the ADPKD is associated with a PKD1 allele or a PKD2 allele.

In some embodiments, the polycystic kidney disease is autosomal recessive polycystic disease (ARPKD).

In some embodiments, a therapeutically effective amount of an additional therapeutic or prophylactic agent is administered to the subject.

In some embodiments, the additional therapeutic or prophylactic agent is tolvaptan.

In some embodiments, an additional treatment or medical management is administered to the subject.

In another aspect, the disclosure provides a method of evaluating a biological response to an antibody-therapeutic conjugate, the method comprising:
a) differentiating a first population of stem cells into a first kidney organoid and a second population of stem cells into a second kidney organoid,
b) exposing the first kidney organoid to an antibody-therapeutic conjugate without exposing the second kidney organoid to the antibody-therapeutic conjugate, and
c) comparing the first kidney organoid and the second kidney organoid, thereby evaluating the biological response.

In yet another aspect, the disclosure provides a method of evaluating a biological response to an antibody-therapeutic conjugate, the method comprising:
a) exposing a first population of stem cells to an antibody-therapeutic conjugate without exposing a second population of cells to the antibody-therapeutic conjugate,
b) differentiating the first population of stem cells into a first kidney organoid and differentiating a second population of stem cells into a second kidney organoid, and
c) comparing the first kidney organoid and the second kidney organoid, thereby evaluating the biological response.

In some embodiments, the stem cells are induced pluripotent stem (IPS) cells.

In some embodiments, the IPS cells are generated from a biological sample from the subject.

In yet another aspect, the disclosure provides a method of recovering or restoring an inactivated PKD1 allele or PKD2 allele in a subject in need thereof, the method comprising administering to the subject a therapeutically effective amount of an antibody-therapeutic conjugate, wherein the antibody-therapeutic conjugate comprises:
a) an anti-TACSTD2 antibody or antigen-binding fragment thereof, or
b) a bispecific antibody that binds TACSTD2 and second antigen.

In some embodiments, the antibody-therapeutic conjugate comprises a gene therapy and
a) an anti-TACSTD2 antibody or antigen-binding fragment thereof, or
b) a bispecific antibody that binds TACSTD2 and an additional antigen.

In yet another aspect, the disclosure provides a method of treating or preventing cyst formation, e.g., cyst formation in kidney tissue. In some embodiments, the kidney tissue is pre-cystic. In some embodiments, the kidney tissue is cystic. In some embodiments, the methods disclosed herein may be used to assess, diagnose, or predict cyst formation, e.g., cyst formation in kidney tissue.

In yet another aspect, the disclosure provides a method of reducing, inhibiting, preventing or ablating cystic kidney tissue in a subject in need thereof, the method comprising administering to the subject a therapeutically effective amount of an antibody-therapeutic conjugate, wherein said antibody-therapeutic conjugate comprises:
a) an anti-TACSTD2 antibody or antigen-binding fragment thereof, or
b) a bispecific antibody that binds TACSTD2 and a second antigen.

In some embodiments, the antibody-therapeutic conjugate comprises:
a) an anti-TACSTD2 antibody or antigen-binding fragment thereof, or a bispecific antibody that binds TACSTD2 and an additional antigen, and
b) a topoisomerase I inhibitor, a tubulin polymerization inhibitor, a vasopressin receptor 2 antagonist, an mTOR inhibitor, or a gene therapy.

### Examples of Polypeptides Binding TACSTD2 Protein

In some embodiments, a polypeptide that binds TACSTD2 comprises sacituzumab, also known as HuRS7 or hRS7 (CAS No. 1796566-95-4; see Govindan S V, Stein R, Qu Z, Chen S, Andrews P, Ma H, Hansen H J, Griffiths G L, Horak I D, Goldenberg D M. Preclinical therapy of breast cancer with a radioiodinated humanized anti-EGP-1 monoclonal antibody: advantage of a residualizing iodine radiolabel. Breast Cancer Res Treat. 2004 March; 84 (2): 173-82. Doi: 10.1023/B:BREA.0000018417.02580.ef. PMID: 14999147.). Sacituzumab is a humanized monoclonal antibody with a molecular weight of 150 kilodaltons (kDa).

In some embodiments, a polypeptide that binds TACSTD2 comprises datopotamab (CAS No. 2267989-53-5). Datopotamab is a humanized IgG1 monoclonal antibody.

In some embodiments, a polypeptide that binds TACSTD2 comprises PF-06478924 (also known as RN926, a humanized anti-TACSTD2 IgG1 antibody).

### Examples of Therapeutic Agents and Targets for the Treatment of PKD

In some embodiments, a target for the treatment of PKD comprises a marker of cystic epithelium, e.g., a cyst initiation candidate, a marker low (e.g., present at a low level or expressed at a low level) in normal kidney cells and elevated (e.g., present at an elevated level or expressed at an elevated level) in cyst lining cells.

In some embodiments, a therapeutic agent for the treatment of PKD or ADPKD comprises tolvaptan (CAS No. 150683-30-0; IUPAC name: N-[4-(7-chloro-5-hydroxy-2,3,4,5-tetrahydro-1-benzazepine-1-carbonyl)-3-methylphenyl]-2-methylbenzamide; also known as SAMSCAR and JYNARQUE®), a vasopressin receptor 2 antagonist that is FDA approved for the treatment of autosomal dominant polycystic kidney disease (ADPKD) and hyponatremia.

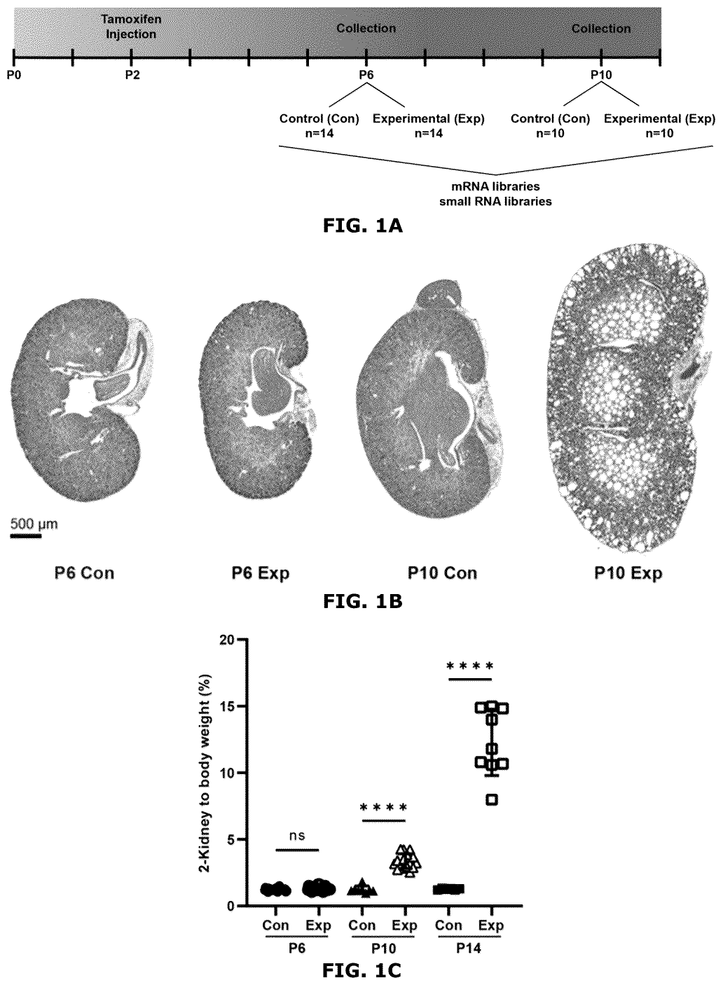

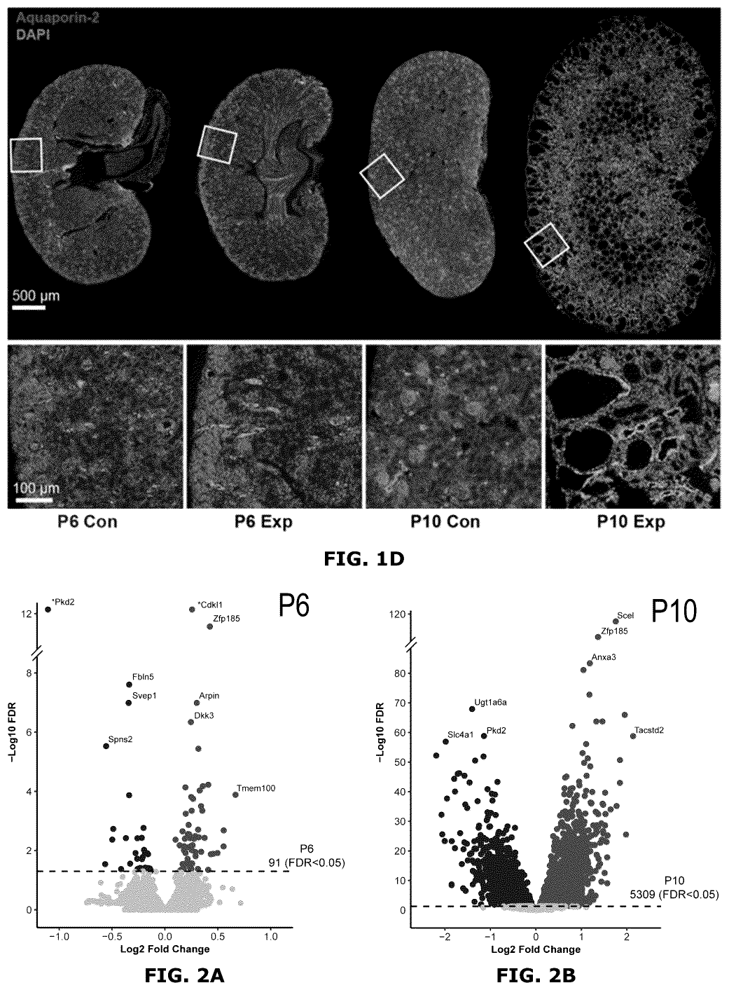

In some embodiments, a therapeutic agent for the treatment of PKD or ADPKD comprises RGLS4326 (CAS No. 2229964-07-0), an oligonucleotide candidate therapy for ADPKD currently undergoing a clinical trial (ClinicalTrials.gov ID NCT05521191). RGLS4326 is a 9-nucleotide modified oligonucleotide that targets the mature micro RNA (miR or miRNA) miR-17. For the structure of RGLS4326, see FIG. 1G of Lee et al. (2019) and accompanying description (55).

In some embodiments, a therapeutic agent for the treatment of PKD or ADPKD comprises metformin (CAS No. 657-24-9; IUPAC name: N,N-Dimethylimidodicarbonimidic diamide; also known as N,N-dimethylbiguanide), an antidiabetic agent sold under the names Glucophage, Riomet, Fortamet, Glumetza, Obimet, Gluformin, Dianben, Diabex, Diaformin, Metsol, Siofor, Metfogamma, and Glifor, including instant release (IR) or extended release (XR) formulations. Metformin is currently being studied for its potential effects on ADPKD (ClinicalTrials.gov ID NCT04939935).

In some embodiments, a therapeutic agent for the treatment of PKD or ADPKD comprises lixivaptan (CAS No. 168079-32-1; IUPAC name: N-[3-chloro-4-(6,11-dihydropyrrolo[2,1-c][1,4]benzodiazepine-5-carbonyl)phenyl]-5-fluoro-2-methylbenzamide; also known as VPA-985), a vasopressin 2 receptor antagonist.

In some embodiments, a therapeutic agent for the treatment of PKD or ADPKD comprises tesevatinib (CAS No. 781613-23-8; IUPAC name: N-(3,4-dichloro-2-fluorophenyl)-6-methoxy-7-{[(3aR,5r,6aS)-2-methyloctahydrocyclopenta[c]pyrrol-5-yl]methoxy}quinazolin-4-amine; also known as KD019, XL647), a multi-kinase inhibitor.

In some embodiments, a therapeutic agent for the treatment of PKD or ADPKD comprises venglustat (CAS No. 1401090-53-6; IUPAC name: [(3S)-1-azabicyclo[2.2.2]octan-3-yl] N-[2-[2-(4-fluorophenyl)-1,3-thiazol-4-yl]propan-2-yl]carbamate; also known as ibiglustat), a glucosylceramide synthase inhibitor.

In some embodiments, a therapeutic agent for the treatment of PKD or ADPKD comprises: epidermal growth factor receptor (EGFR) tyrosine kinase inhibitors EKI-785 and EKB-569, peroxisome proliferator-activated receptor-γ (PPARγ) agonist Rosiglitazone, somatostatin receptor inhibitor Octreotide, phosphodiesterase activators, SRC inhibitor SKI-606, RAF inhibitor Sorafenib, MEK inhibitor PD184352, mTor inhibitor Everolimus, CFTR inhibitor CFTRinh-172, CDK inhibitor Roscovitine, glucosylceramide synthase inhibitor Genz-123346, HMG-COA reductase inhibitor Lovastatin, Triptolide, and Curcumin (see Sun Y, Zhou H, Yang B X. Drug discovery for polycystic kidney disease. Acta Pharmacol Sin. 2011 June; 32 (6): 805-16. doi: 10.1038/aps.2011.29. PMID: 21642949; PMCID: PMC4009953.).

In some embodiments, a therapeutic agent for the treatment of PKD or ADPKD comprises: sodium channel blocker Amiloride, dopamine receptor antagonist Amisulpride, a norepinephrine inhibitor, a norepinephrine reuptake inhibitor, serotonin receptor antagonist, serotonin-norepinephrine reuptake inhibitor (SNRI) Amitriptyline, Dopamine receptor agonist Bromocriptine, Anaplastic lymphoma kinase (ALK) receptor tyrosine kinase inhibitor Crizotinib, Calcium channel blocker Diltiazem, Peroxisome proliferator-activated receptor (PPAR) receptor agonist Fenofibrate, Selective serotonin reuptake inhibitor (SSRI) Fluoxetine, Dopamine receptor antagonist Fluphenazine, Hydroxymethylglutaryl-CoA reductase (HMGCR) inhibitor Fluvastatin, Norepinephrine reuptake inhibitor, serotonin reuptake inhibitor Imipramine, Adrenergic receptor antagonist, serotonin receptor antagonist Mirtazapine, Acetylcholine receptor agonist Nicotine, Tubulin polymerization inhibitor Paclitaxel, Dopamine receptor antagonist Perphenazine, and Hydroxymethylglutaryl-CoA reductase (HMGCR) inhibitor Pravastatin (see Wilk E J, Howton T C, Fisher J L, Oza V H, Brownlee R T, McPherson K C, Cleary H L, Yoder B K, George J F, Mrug M, Lasseigne B N. Prioritized polycystic kidney disease drug targets and repurposing candidates from pre-cystic and cystic mouse Pkd2 model gene expression reversion. Mol Med. 2023 May 22; 29(1):67. doi: 10.1186/s10020-023-00664-z. PMID: 37217845; PMCID: PMC10201779.).

In some embodiments, a therapeutic agent for the treatment of PKD or ADPKD comprises: Birinapant, Gamma-Linolenic acid (Gamolenic Acid), Eicosapentaenoic acid (Icosapent), Meclofenamic Acid, Zileuton, or Indomethacin (see Malas T B, Leonhard W N, Bange H, Granchi Z, Hettne K M, Van Westen G J P, Price L S, 't Hoen P A C, Peters D J M. Prioritization of novel ADPKD drug candidates from disease-stage specific gene expression profiles. EBioMedicine. 2020 January; 51:102585. doi: 10.1016/j.ebiom.2019.11.046. Epub 2019 Dec. 24. PMID: 31879244; PMCID: PMC7000333.).

In some embodiments, a therapeutic agent for the treatment of PKD or ADPKD comprises: CDK1, PRKCB, PRKCZ, PPARD, STAT3, THRA, CCL2, CCR2, SLC1A1, PTGER3, AKR1B10, LGALS3, BIRC2, HSD17B2, P2RX7, PLD2, CYP2J2, MGLL, FKBP4, PTGES, ALOX5AP, P2RY6, AKR1A1, MAPT, CYP51A1, TRAP1, AKR1C1, AKR1C3, AKR1C2, or the encoding gene of any of the aforementioned targets (see Malas T B, Leonhard W N, Bange H, Granchi Z, Hettne K M, Van Westen G J P, Price L S, 't Hoen P A C, Peters D J M. Prioritization of novel ADPKD drug candidates from disease-stage specific gene expression profiles. EBioMedicine. 2020 January; 51:102585. doi: 10.1016/j.ebiom.2019.11.046. Epub 2019 Dec. 24. PMID: 31879244; PMCID: PMC7000333.).

In some embodiments, a therapeutic agent for the treatment of PKD or ADPKD comprises: Hydralazine (CAS No. 86-54-4; IUPAC name: 1-hydrazinylphthalazine; also known as Apresoline, Adrolazine, Apresrex), mTOR inhibitor Rapamycin (CAS No. 53123-88-9, IUPAC name: (1R,9S,12S,15R,16E,18R,19R,21R,23S,24E,26E,28E,30S,32S,35R)-1,18-dihydroxy-12-{(2R)-1-[(1S,3R,4R)-4-hydroxy-3-methoxycyclohexyl]-2-propanyl}-19,30-dimethoxy-15,17,21,23,29,35-hexamethyl-11,36-dioxa-4-azatricyclo[30.3.1.0˜4,9˜]hexatriaconta-16,24,26,28-tetraene-2,3,10,14,20-pentone; also known as Rapamune, Sirolimus), or Everolimus (CAS No. 159351-69-6; IUPAC name: Dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0 hexatriaconta-16,24,26,28-tetraene-2,3,10,14,20-pentone).

### Examples of Therapeutic Antibody-Drug Conjugates (ADCs) Targeting a Marker of Cystic Epithelium

In some embodiments, an ADC targets one or more markers of cystic epithelium, e.g., a cyst initiation candidate, a marker low (e.g., present at a low level or expressed at a low level) in normal kidney cells and elevated (e.g., present at an elevated level or expressed at an elevated level) in cyst lining cells. In some embodiments, an ADC comprises one or more whole antibodies or antigen-binding fragments thereof that bind a marker of cystic epithelium. In some embodiments, a marker of cystic epithelium is a gene, or a gene product (e.g., RNA, protein) associated with a gene selected from polycystin 2, transient receptor potential cation channel (PKD2), tumor associated calcium signal transducer 2 (TACSTD2), BTB domain and CNC homolog 1 (BACH1), A-kinase anchoring protein 12 (AKAP12), alpha-1,6-mannosylglycoprotein 6-beta-N-acetylglucosaminyltransferase (MGAT5), BTB domain and CNC homolog 2 (BACH2), semaphorin 3C (SEMA3C), sphingomyelin synthase 1 (SGMS1), integrin subunit alpha 9 (ITGA9), dynein axonemal heavy chain 5 (DNAH5), netrin 4 (NTN4), Cdc42 guanine nucleotide exchange factor 9 (ARHGEF9), inositol monophosphatase 1 (IMPA1), zinc finger matrin-type 3 (ZMAT3), solute carrier family 14 member 2 (SLC14A2), ADAM metallopeptidase with thrombospondin type 1 motif 16 (ADAMTS16), EH domain binding protein 1 (EHBP1), F-box protein 25 (FBX025), NGFI-A binding protein 1 (NAB1), ubiquitin conjugating enzyme E2 Z (UBE2Z), sushi, von Willebrand factor type A, EGF and pentraxin domain containing 1 (SVEP1), plexin domain containing 2 (PLXDC2), rhophilin Rho GTPase binding protein 2 (RHPN2), cyclin dependent kinase like 1 (CDKL1), protein tyrosine phosphatase domain containing 1 (PTPDC1), smoothelin like 2 (SMTNL2), solute carrier family 22 member 15 (SLC22A15), allograft inflammatory factor 1 like (AIFIL), solute carrier family 37 member 3 (SLC37A3), and sushi domain containing 6 (SUSD6).

In some embodiments, an ADC that targets TACSTD2 comprises sacituzumab govitecan (CAS No. 1491917-83-9), also known as TRODELVY®, hRS7-SN38, IMMU-132, isactuzumab govitecan, sacituzumab govitecan-hziy, and TROP-2-SN-38. Sacituzumab govitecan is an FDA-approved drug indicated for locally advanced or metastatic breast cancer and locally advanced or metastatic urothelial cancer. Sacituzumab govitecan comprises an anti-TACSTD2 antibody (sacituzumab) and a topoisomerase I inhibitor (SN-38) connected by a linker. There are approximately 7-8 molecules of SN-38 attached to each antibody.

In some embodiments, an ADC that targets TACSTD2 comprises SN-38 (CAS No. 86639-52-3; IUPAC name: (4S)-4,11-Diethyl-4,9-dihydroxy-1,4-dihydro-3H,14H-pyrano[3′,4′:6,7]indolizino[1,2-b]quinoline-3,14-dione; also known as: 7-Ethyl-10-hydroxycamptothecin, govitecan, NK102), a topoisomerase I inhibitor. SN-38 is an active metabolite of the chemotherapeutic agent irinotecan (CAS No. 97682-44-5; IUPAC name: (S)-4,11-diethyl-3,4,12,14-tetrahydro-4-hydroxy-3,14-dioxo1H-pyrano[3′,4′: 6,7]-indolizino[1,2-b]quinolin-9-yl-[1,4′bipiperidine]-1′-carboxylate; also known as CPT-11 and CAMPTOSAR®).

Adverse effects of sacituzumab govitecan can include neutropenia (the most frequent, yet rare, serious reaction), anemia, nausea, vomiting, diarrhea (potentially severe, may case acute kidney injury), alopecia, fatigue, constipation, depletion of electrolytes (magnesium, calcium, phosphate, sodium, and potassium), decreased creatinine clearance, and increased blood glucose levels. Current clinical trials investigating uses for sacituzumab govitecan include TROPHY (ClinicalTrials.gov ID NCT03547973), TROPICS (ClinicalTrials.gov ID NCT03901339) (56), and ASCENT (ClinicalTrials.gov ID NCT02574455).

In some embodiments, an ADC that targets TACSTD2 comprises datopotamab deruxtecan (CAS No. 2238831-60-0), also known as Dato-DXd and DS-1062a. Datopotamab deruxtecan comprises an anti-TACSTD2 antibody (datopotamab) and a topoisomerase I inhibitor (Dxd).

In some embodiments, an ADC that targets TACSTD2 comprises deruxtecan, also known as Dxd (CAS No. 1599440-33-1; IUPAC name: N-[(1S,9S)-9-Ethyl-5-fluoro-2,3,9,10,13,15-hexahydro-9-hydroxy-4-methyl-10,13-dioxo-1H,12H-benzo[de]pyrano[3′,4′: 6,7]indolizino[1,2-b]quinolin-1-yl]-2-hydroxyacetamide; also known as Exatecan derivative for ADC), a DNA topoisomerase I inhibitor.

In some embodiments, an ADC that targets TACSTD2 comprises PF-06664178 (also known as RN926-Aur0101, RN927C). Aur0101 is an inhibitor of tubulin polymerization. PF-06664178 is a cleavable TACSTD2 antibody-therapeutic conjugate or anti-drug conjugate (ADC) (RN927C) with a microtubule inhibitor (MTI) linker-payload, PF-06380101 (62).

In some embodiments, an ADC that targets TACSTD2 comprises PF-06380101 (also known as AcLys-VCAur0101), an inhibitor of tubulin polymerization.

### Example Compositions of Antibody-Therapeutic Conjugates for the Treatment of PKD

In some embodiments, an antibody-therapeutic conjugate for the treatment of PKD or ADPKD comprises a bispecific antibody. In some embodiments, the bispecific antibody targets TACSTD2. In some embodiments, the bispecific antibody targets TACSTD2 and a cell surface marker that is upregulated in PKD or ADPKD. In some embodiments, the bispecific antibody targets TACSTD2 and a kidney-specific cell surface marker. In some embodiments, the kidney-specific cell surface marker is a collecting duct marker or a renal tubule marker.

In some embodiments, an antibody-therapeutic conjugate for the treatment of PKD or ADPKD comprises a gene therapy. In some embodiments, an antibody-therapeutic conjugate for the treatment of PKD or ADPKD comprises an anti-TACSTD2 antibody and a gene therapy. In some embodiments, the gene therapy targets: CDK1, PRKCB, PRKCZ, PPARD, STAT3, THRA, CCL2, CCR2, SLC1A1, PTGER3, AKRIB10, LGALS3, BIRC2, HSD17B2, P2RX7, PLD2, CYP2J2, MGLL, FKBP4, PTGES, ALOX5AP, P2RY6, AKRIAI, MAPT, CYP51A1, TRAP1, AKR1C1, AKRIC3, or AKRIC2 (see Malas T B, Leonhard W N, Bange H, Granchi Z, Hettne K M, Van Westen G J P, Price L S, 't Hoen P A C, Peters D J M. Prioritization of novel ADPKD drug candidates from disease-stage specific gene expression profiles. EBioMedicine. 2020 January; 51:102585. doi: 10.1016/j.ebiom.2019.11.046. Epub 2019 Dec. 24. PMID: 31879244; PMCID: PMC7000333.).

In some embodiments, a gene therapy comprises a clustered regularly interspaced short palindromic repeats (CRISPR)/CRISPR-associated protein 9 (Cas9) editing system, a transcription activator-like effector nuclease (TALEN) editing system, a zinc-finger nuclease (ZFN) editing system, a homing endonuclease system, or a meganuclease system. In some embodiments, a gene therapy comprises one or more guide RNAs (gRNA) or a DNA-binding domains. In some embodiments, the one or more gRNAs or DNA-binding domains direct the editing system to the locus of a gene. In some embodiments, the gene is PKD1 or PKD2. In some embodiments, the gene is PKHD1. In some embodiments, the gene is TACSTD2. In some embodiments, the gene is CDK1, PRKCB, PRKCZ, PPARD, STAT3, THRA, CCL2, CCR2, SLC1A1, PTGER3, AKR1B10, LGALS3, BIRC2, HSD17B2, P2RX7, PLD2, CYP2J2, MGLL, FKBP4, PTGES, ALOX5AP, P2RY6, AKR1A1, MAPT, CYP51A1, TRAP1, AKR1C1, AKR1C3, or AKR1C2.

In some embodiments, an antibody-therapeutic conjugate for the treatment of PKD or ADPKD comprises a topoisomerase I inhibitor, a tubulin polymerization inhibitor, a vasopressin receptor 2 antagonist, or an mTOR inhibitor. In some embodiments, an antibody-therapeutic conjugate for the treatment of PKD or ADPKD comprises a therapeutic protein (68).

In some embodiments, an antibody-therapeutic conjugate for the treatment of PKD or ADPKD comprises SN38, deruxtecan (DXd), Aur0101, tolvaptan, or rapamycin.

In some embodiments, an antibody-therapeutic conjugate for the treatment of PKD or ADPKD comprises sacituzumab govitecan (hRS7-SN38), datopotamab deruxtecan (Dato-DXd), or PF-06664178 (RN926-Aur0101).

### Example Methods of Treating PKD and Developing Treatments for PKD

In one aspect, the disclosure herein provides a method of treating a subject in need thereof, comprising administering to the subject a therapeutically effective amount of an antibody-therapeutic conjugate, wherein the subject has or is predisposed to have a polycystic kidney disease (PKD). In some embodiments, the subject presents one or more symptoms of PKD. In some embodiments, the subject does not present symptoms of PKD. In some embodiments, the subject is at risk of developing symptoms of PKD.

In some embodiments, the antibody-therapeutic conjugate comprises a whole antibody or an antigen-binding fragment thereof. In some embodiments, an antibody comprises a whole antibody, an intact antibody, a murine antibody, a human antibody, or a humanized antibody. In some embodiments, an antibody (e.g., a human or humanized antibody) is an IgA, IgD, IgE, IgG, or IgM antibody. In some embodiments, an antibody (e.g., a human or humanized antibody) is an IgG1, IgG2, IgG3, or IgG4 antibody.

In some embodiments, the antibody-therapeutic conjugate comprises an anti-TACSTD2 antibody or antigen-binding fragment thereof. In some embodiments, the antibody-therapeutic conjugate comprises an antibody selected from the group comprising sacituzumab, datopotamab, and RN926. In some embodiments, the antibody-therapeutic conjugate comprises a different TACSTD2-binding polypeptide, antibody, or antigen-binding fragment thereof.

In some embodiments, the antibody-therapeutic conjugate comprises a bispecific antibody. In some embodiments, the antibody-therapeutic conjugate comprises a bispecific antibody that binds TACSTD2 and an additional antigen. In some embodiments, an additional antigen comprises a cell surface protein or glycoprotein of a renal collecting duct cell, a renal proximal tubule cell, or a glomerular vesical epithelial cell. In some embodiments, an additional antigen comprises Aquaporin-2 (AQP-2).

In some embodiments, the antibody-therapeutic conjugate comprises a therapeutic agent.

In some embodiments, the antibody-therapeutic conjugate comprises a cytotoxic drug, a cytostatic drug, or a gene therapy. In some embodiments, the antibody-therapeutic conjugate comprises a therapeutic agent that exhibits cytotoxic and/or cytostatic properties.

Examples of cytotoxic drugs can include, but are not limited to, carcinogens and teratogens. In some embodiments, a cytotoxic drug is a chemotherapeutic agent or a targeted therapy agent. Examples of cytotoxic drugs can include, but are not limited to, kinase inhibitors such as sunitinib and imatinib, and immunomodulatory drugs such as thalidomide and lenalidomide.

In some embodiments, a cytostatic drug is a chemotherapeutic agent or a targeted therapy agent. Examples of cytostatic drugs can include, but are not limited to, progestins such as dienogest, megestrol acetate, and levonorgestrel.

In some embodiments, the gene therapy comprises gene editing, gene augmentation, gene replacement, or gene addition. In some embodiments, a gene therapy corrects one or more pathological alleles or portions thereof. In some embodiments, a gene therapy replaces a pathological allele or portion thereof. In some embodiments, a gene therapy adds a functional allele or portion thereof. In some embodiments, a gene therapy deletes a pathological allele or portion thereof.

In some embodiments, the gene therapy targets PKD1 or PKD2. In some embodiments, the gene is PKHD1.

In some embodiments, the gene therapy comprises an anti-sense oligonucleotide (ASO) or small interfering RNA (siRNA). In some embodiments, a gene therapy interferes with transcription of a gene. In some embodiments, a gene therapy inactivates transcription of a gene. In some embodiments, a gene therapy interferes with translation of a transcript. In some embodiments, a gene therapy leads to degradation of a transcript.

In some embodiments, the gene therapy targets TACSTD2. In some embodiments, a gene therapy targets a gene that is differentially expressed in ADPKD or a model thereof.

In some embodiments, the gene therapy comprises a viral vector or a non-viral delivery system. In some embodiments, a gene therapy comprises a lentiviral vector, a retroviral vector, an adenoviral vector, an adeno-associated viral vector, a herpes viral vector, a pox viral vector, or a human foamy viral vector delivery system. In some embodiments, a gene therapy comprises a mechanical or chemical delivery system. In some embodiments, a gene therapy delivery system comprises electroporation, sonoporation, a nanoparticle, a peptide, a lipid emulsion, or a polymer vector.

In some embodiments, the antibody-therapeutic conjugate comprises a topoisomerase I inhibitor, a tubulin polymerization inhibitor, a vasopressin receptor 2 antagonist, or an mTOR inhibitor. In some embodiments, the antibody-therapeutic conjugate comprises a phosphodiesterase activator, an epidermal growth factor receptor (EGFR) tyrosine kinase inhibitor, a peroxisome proliferator-activated receptor-γ (PPARγ) agonist, a somatostatin receptor inhibitor, an SRC inhibitor, a RAF inhibitor, a MEK Inhibitor, a CFTR inhibitor, a CDK inhibitor, a glucosylceramide synthase inhibitor, or a HMG-COA reductase inhibitor. In some embodiments, the antibody-therapeutic conjugate comprises a sodium channel blocker, a dopamine receptor antagonist, a norepinephrine inhibitor, a norepinephrine reuptake inhibitor, serotonin receptor antagonist, a serotonin-norepinephrine reuptake inhibitor (SNRI), a dopamine receptor agonist, an anaplastic lymphoma kinase (ALK) receptor tyrosine kinase inhibitor, a calcium channel blocker, a peroxisome proliferator-activated receptor (PPAR) receptor agonist, a selective serotonin reuptake inhibitor (SSRI), a dopamine receptor antagonist, a hydroxymethylglutaryl-CoA reductase (HMGCR) inhibitor, a norepinephrine reuptake inhibitor, a serotonin reuptake inhibitor, an adrenergic receptor antagonist, a serotonin receptor antagonist, an acetylcholine receptor agonist, a tubulin polymerization inhibitor, a dopamine receptor antagonist, or a hydroxymethylglutaryl-CoA reductase (HMGCR) inhibitor. In some embodiments, the antibody-therapeutic conjugate comprises a therapeutic agent selected from the group comprising SN38, DXd, Aur0101, tolvaptan, and rapamycin. In some embodiments, the antibody-therapeutic conjugate comprises metformin, lixivaptan, tesevatinib, venglustat, EKI-785, EKB-569, Rosiglitazone, Octreotide, SKI-606, Sorafenib, PD184352, Everolimus, CFTRinh-172, Roscovitine, Genz-123346, Lovastatin, Triptolide, or Curcumin. In some embodiments, the antibody-therapeutic conjugate comprises Amiloride, Amisulpride, Amitriptyline, Bromocriptine, Crizotinib, Diltiazem, Fenofibrate, Fluoxetine, Fluphenazine, Fluvastatin, Imipramine, Mirtazapine, Nicotine, Paclitaxel, Perphenazine, or Pravastatin.

In some embodiments, the antibody-therapeutic conjugate comprises sacituzumab govitecan (hRS7-SN38), datopotamab deruxtecan (Dato-DXd), or PF-06664178 (RN926-Aur0101).

In some embodiments, the polycystic kidney disease is autosomal dominant polycystic kidney disease (ADPKD). In some embodiments, the ADPKD is associated with a PKD1 allele or a PKD2 allele. In some embodiments, a PKD1 allele or a PKD2 allele contains a pathogenic variant. In some embodiments, a PKD1 allele or a PKD2 allele contains a splicing, frameshift, nonsense, or substitution mutation.

In some embodiments, the polycystic kidney disease is autosomal recessive polycystic disease (ARPKD). In some embodiments, the ARPKD is associated with a PKHD1 allele. In some embodiments, the PKHD1 allele contains a pathogenic variant.

In some embodiments, a therapeutically effective amount of an additional therapeutic or prophylactic agent is administered to the subject. In some embodiments, an additional therapeutic or prophylactic agent is administered to the subject sequentially before or after administration of an antibody-therapeutic conjugate, simultaneously with an antibody-therapeutic conjugate, in the same composition as an antibody-therapeutic conjugate, e.g., in the same pharmaceutical composition as an antibody-therapeutic conjugate, or the like.

In some embodiments, the additional therapeutic or prophylactic agent is tolvaptan.

In some embodiments, an additional treatment or medical management is administered to the subject. In some embodiments, an additional treatment is administered to the subject. In some embodiments, a medical management is administered to the subject.

In some embodiments, an additional treatment is for PKD. In some embodiments, an additional treatment (e.g., dialysis, kidney transplant) is for a complication of PKD (e.g., reduced kidney function or kidney failure) or a side effect (e.g., liver injury) of treatment of PKD (e.g., tolvaptan therapy). Appropriate additional treatments relating to PKD and PKD therapy are well-known by those of skill in the art.

In some embodiments, medical management comprises a prophylactic measure related to an anticipated complication or side effect of PKD treatment. In some embodiments, medical management comprises a prophylactic measure, treatment, or both related to a comorbidity (e.g., diabetes) that is not necessarily caused by PKD. In some embodiments, a comorbidity is not necessarily caused by PKD but may negatively impact PKD (e.g., hypertension). In some embodiments, medical management comprises a prophylactic measure, treatment, or both (e.g., a dietary intervention to reduce hypertension, stress on kidneys, or both) related to a secondary diagnosis, symptom, or effect of PKD (e.g., pain).

In another aspect, the disclosure provides a method of evaluating a biological response to an antibody-therapeutic conjugate, the method comprising:
a) differentiating a first population of stem cells into a first kidney organoid and a second population of stem cells into a second kidney organoid,
b) exposing the first kidney organoid to an antibody-therapeutic conjugate without exposing the second kidney organoid to the antibody-therapeutic conjugate, and
c) comparing the first kidney organoid and the second kidney organoid, thereby evaluating the biological response,
   wherein the first population of stem cells and second population of stem cells are of the same cell line, from the same biological sample donor, or the like. In some embodiments, comparing the first kidney organoid and the second kidney organoid is repeated. In some embodiments, comparing the first kidney organoid and the second kidney organoid is repeated a sufficient number of times (e.g. a sufficiently large sample size and/or a sufficient number of biological and/or technical replicates) such that the results of said comparing may reach statistical significance.

In yet another aspect, the disclosure provides a method of evaluating a biological response to an antibody-therapeutic conjugate, the method comprising:
a) exposing a first population of stem cells to an antibody-therapeutic conjugate without exposing a second population of cells to the antibody-therapeutic conjugate,
b) differentiating the first population of stem cells into a first kidney organoid and differentiating a second population of stem cells into a second kidney organoid, and
c) comparing the first kidney organoid and the second kidney organoid, thereby evaluating the biological response,
   wherein the first population of stem cells and second population of stem cells are of the same cell line, from the same biological sample donor, or the like. In some embodiments, comparing the first kidney organoid and the second kidney organoid is repeated. In some embodiments, comparing the first kidney organoid and the second kidney organoid is repeated a sufficient number of times (e.g. a sufficiently large sample size and/or a sufficient number of biological and/or technical replicates) such that the results of said comparing may reach statistical significance.

As described herein, the step of comparing the first kidney organoid and the second kidney organoid (i.e., the organoids) may be accomplished by qualitative or quantitative means known to those of skill in the art. For example, the organoids or sections (i.e., histological sections) thereof may be stained by hematoxylin and eosin (H&E) for assessment of gross structures, or according to immunohistochemistry (IHC), imuunocytochemistry (ICC), or immunofluorescence (IF) staining methods to assess levels of one or more specific markers (see, e.g., Example 4 Materials and Methods). In some embodiments, comparing may involve comparing levels of one or more transcripts (e.g., via PCR, qPCR, RNA-seq; see, e.g., Example 1 Materials and Methods) or levels of one or more proteins (e.g., via Western blot or staining).

In some embodiments, the stem cells are induced pluripotent stem (IPS) cells.

In some embodiments, the IPS cells are generated from a biological sample from the subject. In some embodiments, the IPS cells are generated from a biological sample from a donor that is compatible with the subject. In some embodiments, the biological sample comprises, as non-limiting examples, a skin sample, a blood sample, a cheek swab, or a urine sample. In some embodiments, the sample includes, as non-limiting examples, peripheral blood mononuclear cells from a blood sample, buccal cells from a cheek swab, fibroblasts from a skin biopsy, keratinocytes from a hair follicle, or renal epithelial cells from a urine sample.

In yet another aspect, the disclosure provides a method of recovering or restoring an inactivated PKD1 allele or PKD2 allele in a subject in need thereof, the method comprising administering to the subject a therapeutically effective amount of an antibody-therapeutic conjugate, wherein the antibody-therapeutic conjugate comprises:
a) an anti-TACSTD2 antibody or antigen-binding fragment thereof, or
b) a bispecific antibody that binds TACSTD2 and second antigen.

In some embodiments, the antibody-therapeutic conjugate comprises a gene therapy and
a) an anti-TACSTD2 antibody or antigen-binding fragment thereof, or
b) a bispecific antibody that binds TACSTD2 and an additional antigen.

In yet another aspect, the disclosure provides a method of reducing or ablating cystic kidney tissue in a subject in need thereof, the method comprising administering to the subject a therapeutically effective amount of an antibody-therapeutic conjugate, wherein said antibody-therapeutic conjugate comprises:
a) an anti-TACSTD2 antibody or antigen-binding fragment thereof, or
b) a bispecific antibody that binds TACSTD2 and a second antigen.

In some embodiments, the antibody-therapeutic conjugate comprises:
a) an anti-TACSTD2 antibody or antigen-binding fragment thereof, or a bispecific antibody that binds TACSTD2 and an additional antigen, and
b) a topoisomerase I inhibitor, a tubulin polymerization inhibitor, a vasopressin receptor 2 antagonist, an mTOR inhibitor, or a gene therapy.

### EXEMPLIFICATION

### Introduction

Polycystic kidney disease (PKD) is an important cause of end stage renal disease, but treatment options are limited. While later stages of the disease have been extensively studied, mechanisms driving the initial conversion of a renal tubule into a cyst are not understood. To identify factors that promote the initiation of cysts, the polycystin-2 (Pkd2) gene was deleted and transcriptional changes were surveyed before and just after cysts were evident. This work identified 74 genes that were termed cyst initiation candidates (CICs). The CICs were compared to single cell RNA data from mouse and human kidney to identify epithelial-expressed genes as cyst formation initiates in these cells. Tumor-associated calcium signal transducer 2 (Tacstd2) stood out as a gene with increased abundance within days of Pkd2 loss and prior to cystic transformation. TACSTD2 is a single pass transmembrane protein. It is well characterized in cancer where it has been shown to have oncogenic properties, a feature that could drive cyst formation. TACSTD2 expression is low in normal tissue but is highly expressed in many solid tumors. Antibody-drug conjugates targeting TACSTD2 are in clinical use for refractory breast and urothelial cancer. Using tissue biopsies and organoids, this disclosure shows that human TACSTD2 protein is low in normal kidney cells, elevated in cyst lining cells, and provides a cyst-specific target for therapeutic delivery (e.g., for an antibody-drug conjugate such as sacituzumab govitecan).

Autosomal Dominant Polycystic Kidney Disease (ADPKD) is an inherited, progressive disease in which large, fluid-filled cysts grow in both kidneys, ultimately destroying organ function. It is relatively common, affecting approximately 1 in 1,000 people (1-3). ADPKD typically results from inactivating mutations in genes encoding either of two transmembrane proteins, polycystin-1 (PKD1 in humans, Pkd1 in mice) or polycystin-2 (PKD2/Pkd2). The precise function of these channel-like proteins is unknown, but they are thought to regulate proliferation and differentiation to maintain tubular architecture. Numerous pathways downstream of the polycystins have been identified, including cAMP signaling, which is the basis of Tolvaptan, the only FDA-approved drug therapy for ADPKD (4,5). The beneficial effects of Tolvaptan therapy are modest and accompanied by significant side effects, such as aquaresis and risk of liver damage (4); accelerated disease progression following discontinuation of use (54); and prohibitively high cost (52). Nonetheless, most patients require dialysis or kidney transplant by late adulthood after reaching end stage renal disease (6). There are no FDA-approved therapies for ADPKD in adolescents, children, or infants, although Tolvaptan has been tested in limited cohorts (51, 53).

While ADPKD is categorized as an adult disease (7), cyst formation begins in utero and continues throughout an individual's lifespan. Adolescents may experience early symptoms of hypertension and pain, and symptom prevalence increases with age and cyst burden despite preserved kidney function (8,9). Ultimately cyst burden overwhelms kidney function, leading to organ failure.

While the later stages of the disease have been extensively studied, there has been limited research into the early stages of ADPKD, including the mechanism of cyst initiation. In some embodiments, drivers of cyst formation have been identified, which could provide additional therapeutic targets. In this work, RNA sequencing was used to detect the earliest transcriptional changes after Pkd2 deletion in mouse. Seventy-four differentially expressed cyst initiation candidates (CICs) were identified, including Tacstd2/TACSTD2, also known as Trop2. CICs were cross-referenced with published data from pre-cystic mouse models and single-cell RNA sequence from murine and human kidneys. While unstudied in the context of ADPKD, TACSTD2 is highly expressed in metastatic breast cancer and other epithelial tumors. It is targeted by sacituzumab govitecan (TRODELVY®, Immunomedics/Gilead Sciences, Foster City, CA), an antibody-drug conjugate, as a treatment for aggressive cancer (10). Tacstd2 antibodies strongly label small cysts in both human and mouse kidneys with minimal label in non-cystic tubules.

### Discussion

Improving ADPKD treatment and outcomes depends on understanding genetic changes that promote cyst development. Focusing on meaningful changes in kidney gene expression that precede cyst formation has been challenging due to low amplitude gene expression within low abundance cell types in heterogeneous tissues. Previous investigations (14) found few genes differentially expressed in pre-cystic time points but found many differentially expressed genes later disease stages. However, the genes elevated in grossly cystic tissue generally participate in broad proliferative pathways, and do not elucidate mechanisms of cyst initiation.

The present work set out to identify potential cyst drivers by controlling the timing of Pkd2 loss. To find low amplitude changes present before gross cyst formation, a high number of animals with equal sex parity were utilized. The data were then cross-referenced to previous studies to find genes with consistent changes. The differentially expressed genes were further cross-referenced against human disease and murine single cell atlases to focus on epithelial genes. Through the disclosed multi-species, multi-omic approach, 74 cyst initiation candidates were identified, including tumor-associated calcium signal transducer 2 (Tacstd2).

The chosen model had systemic Pkd2 deletion and cyst initiation in young, developing kidneys. While ADPKD is normally thought of as an adult-onset disease, cysts initiate in utero and the extent of early cysts is likely a driver of disease severity (64-67). This model offers valuable insights into human disease. To bolster the disclosed findings, original data presented herein was cross-referenced with high-quality published research. In some embodiments, the role of Tacstd2 as a marker of cystic epithelium is beneficial for targeting anti-cyst therapies to affected tissues.

Robust elevation of Tacstd2 was observed in Woo et al. (14), but not in Kunnen et al. (22) or Zhang et al. (23). A major difference between the studies was the age at which polycystin was deleted. In both Kunnen et al. and Zhang et al., the animals were mature at the time of deletion, while in the present study and the Woo et al. study, the animals lost polycystin in the early postnatal stage when mouse kidneys were still developing. Humans with ADPKD mutations lack polycystin during kidney development in utero. It is possible that the role of Tacstd2/TACSTD2 in cyst initiation may depend on developmental timing. The discrepancies between studies highlight the importance of using an array of genetic models, targets, and time points to elucidate cyst initiators.

To verify that Tacstd2 is a bona fide epithelial cyst initiating candidate, tissues were immunostained for Tacstd2. Consistent with the disclosed RNAseq study, staining was found to be slightly elevated in experimental (Pkd2 deleted) kidneys compared to controls at postnatal day 6 (P6) and dramatically elevated at postnatal day 10 (P10). Mouse findings were consistent with human kidney tissues and organoids, suggesting that TACSTD2 is a relevant target in human disease.

TACSTD2, also known as trophoblast cell surface antigen 2 (TROP2), is a transmembrane protein originally identified in trophoblast cells (24) and subsequently found to be highly expressed in many solid tumors (25). Tacstd2 expression is generally low in most tissues but is seen in cells with stem cell properties (25). It appears to be an oncogene as overexpression increases proliferation of cancer cells with low basal expression and knockdown with siRNA reduces growth of cancer cells with high expression (26). Its ability to mark cancer cells has led to the development of antibody-drug conjugate therapies to deliver cytotoxic payloads to cancer cells (10). The FDA-approved drug sacituzumab govitecan (TRODELVY®), which is a conjugate including a topoisomerase I inhibitor, is in clinical use to treat metastatic triple-negative breast cancer and advanced or metastatic urothelial carcinoma. Side effects of nausea, fatigue, alopecia, febrile neutropenia, myelosuppression, and diarrhea leading to dehydration and acute kidney injury were observed, but other kidney toxicities were not reported. A second drug, datopotamab deruxtecan, is being explored in several clinical trials and has shown reduced side effects. Kidney effects were not reported (27). As described herein, TACSTD2 expression in relation to ADPKD was explored. While antibody-drug conjugates to topoisomerase inhibitors may be too toxic for long term use in PKD patients, adapting the conjugates to carry anti-cyst payloads such as rapamycin could increase the efficacy of an anti-cyst drug while reducing its off-target effects.

Two mouse Tacstd2 mutant lines were generated as disclosed herein. Contrary to expectations, the mice were viable and in the first report, no pathology was observed (28). The second line generated by the Knockout Mouse Program (KOMP) showed microphthalmia and small testis but no other phenotypes (www.mousephenotype.org/data/genes/MGI:1861606). Humans with pathological variants in TACSTD2 develop gelatinous drop-like corneal dystrophy, which is a degenerative disease of the cornea (29). In the cornea, TACSTD2 is thought to bind to claudins 1 and 7 to maintain the structure of the tight junctions. When TACSTD2 is defective, the cornea becomes more permeable and accumulates amyloid, ultimately leading to vision loss (30). The relatively mild phenotypes of mice and humans lacking TACSTD2 support the idea that this gene product could be targeted without causing major off target effects.

In the context of PKD, it is interesting to note that antibodies binding the extracellular domain of Tacstd2 transiently increase cytoplasmic calcium concentration, possibly from an intracellular store (31). The cytoplasmic tail of Tacstd2 contains a PIP2 binding site and numerous pathways are affected by Tacstd2. In tumor cells, AKT signaling shows strong correlation with Tacstd2 expression (32), but MAPK/ERK, ErbB, TGFβ, Wnt/β-catenin, JAK/STAT, and integrin signaling have all been implicated as downstream targets of Tacstd2 (33). Wnt/β-catenin signaling is particularly interesting as this pathway is disturbed in PKD. B-catenin binds directly to Tacstd2 and Tacstd2 over expression promoted nuclear accumulation of β-catenin (34). This observation could have implications for driving cyst formation in PKD as expression of activated β-catenin is sufficient to drive cyst formation (35).

In conclusion, the work disclosed herein demonstrates that Tacstd2 is elevated early in cystic kidney tissue as well as human polycystic kidneys. The insights gained from studies of breast and other aggressive cancers suggest that Tacstd2 is a promoter of cell proliferation and could play a key role in cyst formation. The work described herein suggests that TACSTD2 should be explored as a therapeutic target and that patients with PKD who receive anti-TACSTD2 therapy should be monitored for kidney effects.

### Data and Materials Availability

The data discussed in this disclosure have been deposited in NCBI's Gene Expression Omnibus (49) and are accessible through GEO Series accession numbers: GSE222610 (www.ncbi.nlm.nih.gov/geo/query/acc.cgi?acc=GSE222610) and GSE220322 (www.ncbi.nlm.nih.gov/geo/query/acc.cgi?acc=GSE220322).

### Example 1

Identifying Early Transcriptional Changes after Pkd2 Deletion

### Materials and Methods

### Mouse Studies

Mouse strains used include the following:

Pkd2^{flox/+}. The Pkd2^{flox11-13Δneo} strain (abbreviated as Pkd2^{flox} or Pkd2^{flox/+} throughout the disclosure; RRID:IMSR_JAX:017292; B6.129X1 (Cg)-Pkd2^{tm1.1Tjwt}/J), developed by Garcia-Gonzalez et al. (11), has one functional wild-type Pkd2 allele and one functional floxed Pkd2 allele (exons 11-13 are flanked by loxP sites). Heterozygous Pkd2^{flox/+} animals allow for conditional (Cre-inducible) knockout of one functional Pkd2 allele.

Cagg-Cre^{ER}. The CAGGCre-ER™ strain (abbreviated as Cagg-Cre^{ER} throughout the disclosure), developed by Hayashi and McMahon (36), exhibits tamoxifen-inducible expression of Cre recombinase.

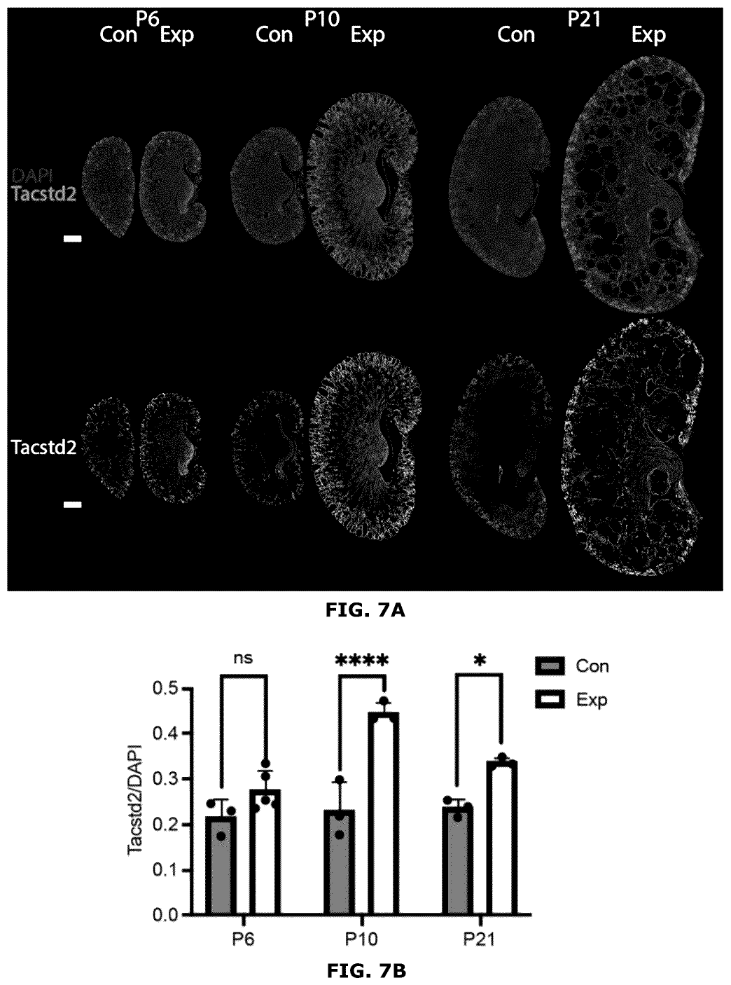

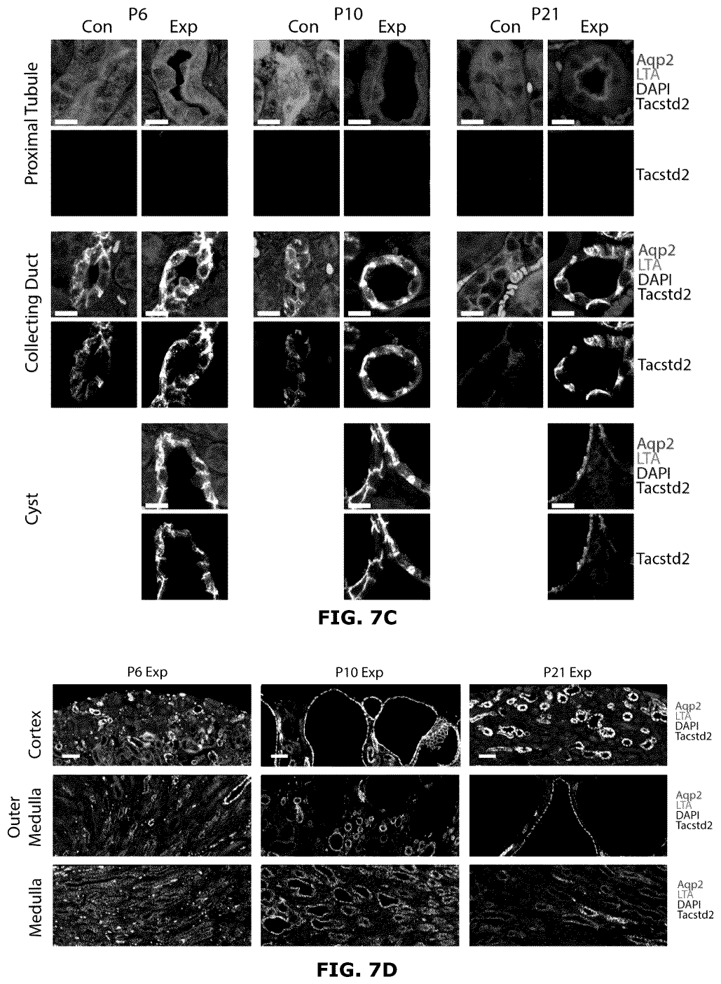

Rosa26-Cre^{ERT}. The Rosa26-Cre^{ERT} strain (RRID:IMSR_JAX:004847; B6; 129-Gt(ROSA)26Sor^{tm1(cre/ERT)Nat}/J), developed by Badea et al. (37), exhibits tamoxifen-inducible expression of Cre recombinase from the Rosa26 safe harbor locus. This strain was only used to generate postnatal day 21 (P21) data shown in FIGS. 7A-7D.

Stra8-Cre. The germline Stra8-Cre strain, developed by Sadate-Ngatchou et al. (38), constitutively expresses Cre recombinase only in premeiotic spermatozoa.

Control, intermediate, and experimental mouse strain crosses used include the following.

Pkd2^{null/+}. The Pkd2^{flox/+} strain was converted to a Pkd2^{null/+} strain by crossing Pkd2^{flox} to the germline Stra8-Cre strain to delete floxed exons 11-13, thereby generating Pkd2^{null}:Stra8-Cre mice. Pkd2^{null}:Stra8-Cre mice were crossed to wild type C57BL/6J mice to remove Stra8-Cre, producing pups with the genotype Pkd2^{null/+}.

Pkd2^{null/+}:Cagg-Cre^{ER}. Pkd2^{null/+} animals were crossed to the Cagg-Cre^{ER} strain to produce Pkd2^{null/+}:Cagg-Cre^{ER} animals. These animals carry one functional wild type Pkd2 allele (Pkd2⁺), one non-functional (Δ11-13) Pkd2 allele (Pkd2^{null}), and the tamoxifen-inducible Cagg-Cre^{ER} recombinase.

Pkd2^{flox/flox}. Pkd2^{flox/+} animals were bred together to produce Pkd2^{flox/flox} animals.

To generate experimental and control animals, Pkd2^{null/+}:Cagg-Cre^{ER} animals were crossed to Pkd2^{flox/flox} animals, producing offspring with the genotypes listed below. After intraperitoneal injection of tamoxifen on post-natal day 2, Cagg-Cre^{ER} converted the Pkd2^{flox} allele to a Pkd2^{null} allele, thereby removing all functional copies of Pkd2 from animals with the Pkd2^{null/flox}:Cagg-Cre^{ER} genotype. The other three possible genotypes retained at least one functional Pkd2 allele (either Pkd2⁺ or Pkd2^{flox}).

Pkd2^{null/flox}:Cagg-Cre^{ER} (experimental animals)

Pkd2^{null/flox} (control animals)

Pkd2^{flox/+}:Cagg-Cre^{ER} (control animals)

Pkd2^{flox/+} (control animals)

To generate experimental animals for postnatal day 21 (P21) data shown in FIGS. 7A-7D, Rosa26-Cre^{ERT}:Pkd2^{flox/flox} pups, generated as described in Smith et al. (20), were nursed by mothers administered with tamoxifen by oral gavage on post-natal days 2, 3, and 4, thereby inducing Cre recombination converting the Pkd2^{flox} alleles to Pkd2^{null} alleles. Recombination removed all functional copies of Pkd2 from Rosa26-Cre^{ERT}:Pkd2^{flox/flox} animals.

All mice were C57BL/6J congenic strains maintained by backcrossing to C57BL/6J (RRID:IMSR_JAX:000664) purchased from Jackson Laboratory (Bar Harbor, ME). These studies were approved by the Institutional Animal Care and Use Committee of the University of Massachusetts Chan Medical School.

mRNA Sequencing

For RNA sequencing, pups segregating Pkd2^{null/flox} and Cagg-Cre^{ER} (i.e., Pkd2^{null/flox}:Cagg-Cre^{ER} pups) were dosed with tamoxifen (50 mg/kg) by intraperitoneal injection on postnatal day 2 (P2). On postnatal day 6 (P6) or day 10 (P10), paired kidneys were immediately frozen in Invitrogen™ RNALATER® Stabilization Solution (Thermo Fisher Scientific), and total RNA was isolated as previously described (39) (FIG. 1A).

Libraries were prepared for 100 bp paired-end sequencing by BGI Genomics (Yantian District, Shenzhen, China). Read quality assessment, alignment and counting were performed using DolphinNext pipeline (40). The modules used were FastQC v0.11.7 (https://www.bioinformatics.babraham.ac.uk/projects/fastqc/) and kallisto v0.46.1 (41). Differential expression analysis was performed using DESeq2 (42). Very low expression genes were excluded using cutoff counts per million (CPM)<2 in at least 48 samples. Inclusion criteria for significant differentially expressed genes (DEGs) consisted of a false discovery rate (FDR)<0.05.

### Small RNA Sequencing

Small RNA libraries were prepared for 50 bp single-end sequencing by BGI Genomics (Yantian District, Shenzhen, China). The sequences were demultiplexed and underwent bar code and adapter sequence removal using the SOAPnuke software (43). The steps for demultiplexing, bar code removal, and adapter removal were carried out by BGI. Read quality assessment, alignment and miRNA prediction and quantification/gene expression calculation as well as differential expression were performed using the omiRas pipeline (44), which uses Bowtie for read mapping (45), mirDeep2 for finding novel miRNA (46), and DESeq for normalizing the data and calculating differential expression (47). Inclusion criteria for significant differentially expressed microRNAs (DEmirs) consisted of raw read counts >0, FDR<0.05, and identification as mature or stem-loop microRNAs.

### Results

To distinguish the earliest transcriptional changes downstream of the loss of Pkd2 function, a conditionally inducible mouse model (11) was utilized (FIG. 1A). After intraperitoneal tamoxifen injection at postnatal day 2 (P2), control (Cagg-Cre^{ER}:Pkd2^{flox/+} or Pkd2^{null/flox}) and experimental (Cagg-Cre^{ER}:Pkd2^{null/flox}) kidneys were studied at postnatal day 6 (P6) and postnatal day 10 (P10) (FIGS. 1A-1C), and postnatal days 14-15 (P14-15) (FIG. 1C). The two-kidney to bodyweight ratio (2K:BW), which is a proxy for cystic burden, was not significantly different between experimental and control animals at P6, but by P10, 2K:BW was significantly elevated in the experimental animals and was further elevated in P14-15 experimental animals (FIG. 1C). Consistent with the 2K:BW, hematoxylin and eosin (H&E) staining revealed no gross differences in kidney architecture in P6 experimental kidneys, but cysts were evident in the P10 experimental kidneys (FIG. 1B). At P10, experimental collecting ducts (marked by Aquaporin-2) were dilated or cystic whereas collecting ducts appeared normal in P6 experimental kidneys (FIG. 1D).

To identify potential drivers of cyst formation, RNA was isolated from P6 (n=28) and P10 (n=20) experimental and control kidneys with sex parity (Table 1). mRNA and small RNA libraries were generated, sequenced, and analyzed by standard pipelines to identify transcriptional changes (FIG. 1A).

TABLE 1
Experimental design

| | PKD2 Control | PKD2 Deletion |
|---|---|---|
| | Pkd2^{flox/+}:Cagg-Cre^{ER} | (Experimental) |
| | (n = 20) or | Pkd2^{null/flox}:Cagg-Cre^{ER} |
| Timepoints | Pkd2^{null/flox} (n = 4) | (n = 24) |
| P6 (n = 28) | Male (n = 7) | Male (n = 7) |
| | Female (n = 7) | Female (n = 7) |
| | Total (n = 14) | Total (n = 14) |
| P10 (n = 20) | Male (n = 3) | Male (n = 5) |
| | Female (n = 7) | Female (n = 5) |
| | Total (n = 10) | Total (n = 10) |

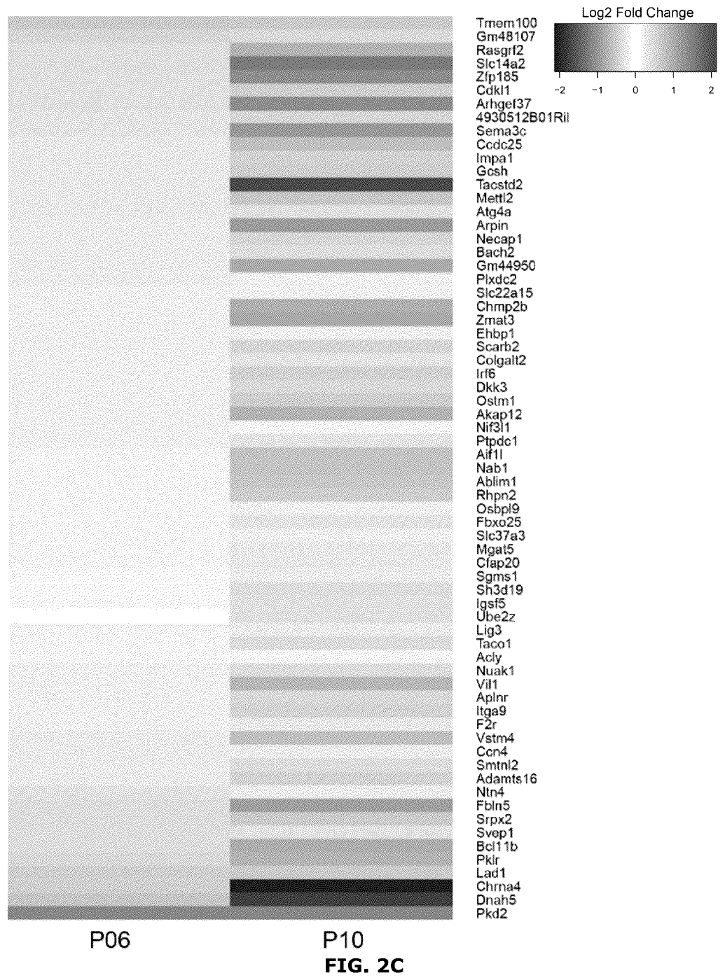

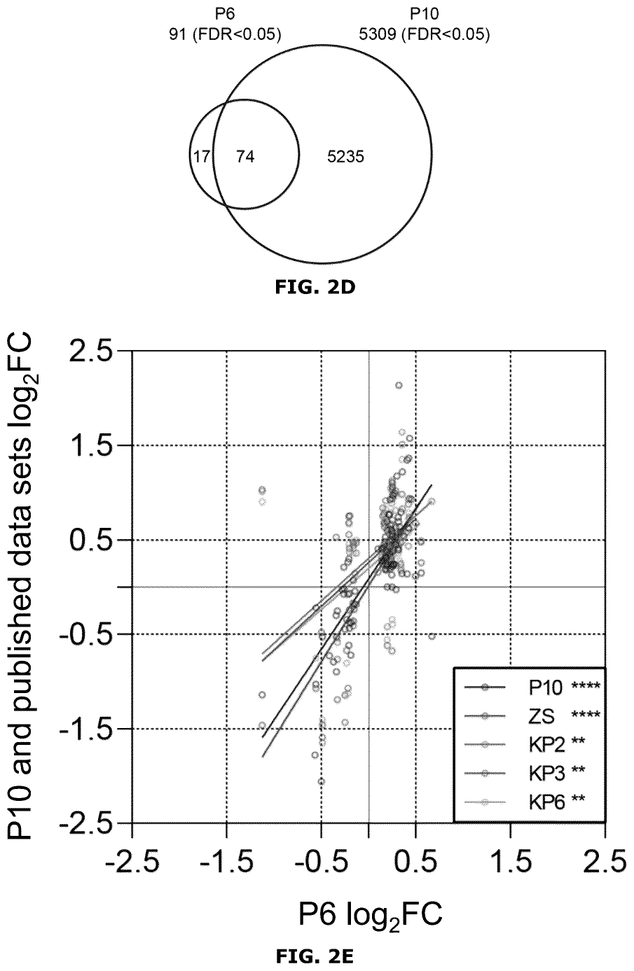

For mRNA, differentially expressed genes (DEGs) were defined as having a false discovery rate (FDR) of less than 0.05. At P6 there were 91 DEGs (59 increased expression and 32 decreased expression) (Table 2, FIG. 2A).

TABLE 2
Top 20 differentially expressed genes with increased
expression (ranked by log₂FC) and Tacstd2 at P6.

| mRNA | log₂FC | FDR | −log₁₀FDR |
|---|---|---|---|
| Tmem100 | 6.67E−01 | 1.31E−04 | 3.88E+00 |
| Aass | 5.55E−01 | 2.08E−03 | 2.68E+00 |
| Gm48107 | 5.54E−01 | 7.25E−03 | 2.14E+00 |
| D630023F18Rik | 4.97E−01 | 1.21E−02 | 1.92E+00 |
| Rasgrf2 | 4.56E−01 | 1.29E−02 | 1.89E+00 |
| Slc14a2 | 4.34E−01 | 1.33E−02 | 1.88E+00 |
| Zfp185 | 4.25E−01 | 3.96E−12 | 1.14E+01 |
| Cdkl1 | 4.21E−01 | 1.17E−27 | 2.69E+01 |
| Arhgef37 | 4.09E−01 | 5.98E−05 | 4.22E+00 |
| Mttp | 4.08E−01 | 4.50E−02 | 1.35E+00 |
| 4930512B01Rik | 3.75E−01 | 3.73E−03 | 2.43E+00 |
| Sema3c | 3.57E−01 | 6.71E−05 | 4.17E+00 |
| Hsd17b11 | 3.53E−01 | 4.52E−04 | 3.35E+00 |
| Ccdc25 | 3.43E−01 | 1.11E−02 | 1.96E+00 |
| Impa1 | 3.41E−01 | 3.16E−04 | 3.50E+00 |
| Gcsh | 3.28E−01 | 9.40E−05 | 4.03E+00 |
| Tacstd2 | 3.20E−01 | 3.59E−03 | 2.45E+00 |
| Mettl2 | 3.16E−01 | 3.64E−06 | 5.44E+00 |
| Atg4a | 3.14E−01 | 1.97E−03 | 2.71E+00 |
| Scn8a | 3.12E−01 | 4.22E−02 | 1.37E+00 |

At P10 there were 5,309 DEGs (2,645 with increased expression and 2,664 with decreased expression) (Table 3, FIG. 2B).

TABLE 3
Top 20 differentially expressed genes with increased
expression (ranked by log₂FC) at P10.

| mRNA | log₂FC | FDR | −log₁₀FDR |
|---|---|---|---|
| Tacstd2 | 2.14E+00 | 1.74E−59 | 5.88E+01 |
| S100a14 | 1.98E+00 | 3.05E−26 | 2.55E+01 |
| Bst1 | 1.95E+00 | 1.12E−66 | 6.60E+01 |
| Egr1 | 1.85E+00 | 2.11E−51 | 5.07E+01 |
| Aldh1a1 | 1.85E+00 | 1.04E−43 | 4.30E+01 |
| S100a6 | 1.77E+00 | 6.86E−36 | 3.52E+01 |
| Scel | 1.76E+00 | 2.11E−111 | 1.11E+02 |
| Col12a1 | 1.64E+00 | 9.73E−35 | 3.40E+01 |
| Slc14a2 | 1.58E+00 | 6.12E−26 | 2.52E+01 |
| Eppk1 | 1.57E+00 | 9.12E−10 | 9.04E+00 |
| Socs3 | 1.57E+00 | 1.52E−18 | 1.78E+01 |
| Wnt7b | 1.55E+00 | 9.50E−24 | 2.30E+01 |
| Krt80 | 1.53E+00 | 3.79E−33 | 3.24E+01 |
| Lgals3 | 1.52E+00 | 2.23E−40 | 3.97E+01 |
| Kcne11 | 1.52E+00 | 2.59E−29 | 2.86E+01 |
| Serpina3g | 1.51E+00 | 3.00E−13 | 1.25E+01 |
| Maff | 1.51E+00 | 3.62E−24 | 2.34E+01 |
| Lrrc75b | 1.49E+00 | 1.57E−26 | 2.58E+01 |
| Golm1 | 1.48E+00 | 2.70E−36 | 3.56E+01 |
| Ntm | 1.48E+00 | 2.16E−27 | 2.67E+01 |

Pkd2 mRNA was significantly reduced at both time points, validating the inducible Cre system. The large difference between the number of DEGs at P10 vs. P6 likely corresponds to the observed rapid cyst development within this time frame.

The analysis described herein indicated that the P10 timepoint with gross cystic changes and many differentially expressed genes would identify gene expression patterns related to downstream cyst-related changes rather than cyst initiation. P6 differences, although fewer and of smaller magnitude than P10, were more likely to represent cyst initiation. Seventy-four genes in the intersection between P6 and P10 DEGs were identified. This list was defined as cyst initiation candidates (CICs) (FIGS. 2C, 2D). The vast majority of CICs are regulated in the same direction at P6 and P10, suggesting that the changes detected at P6 increase with cyst development (FIGS. 2C, 2E).

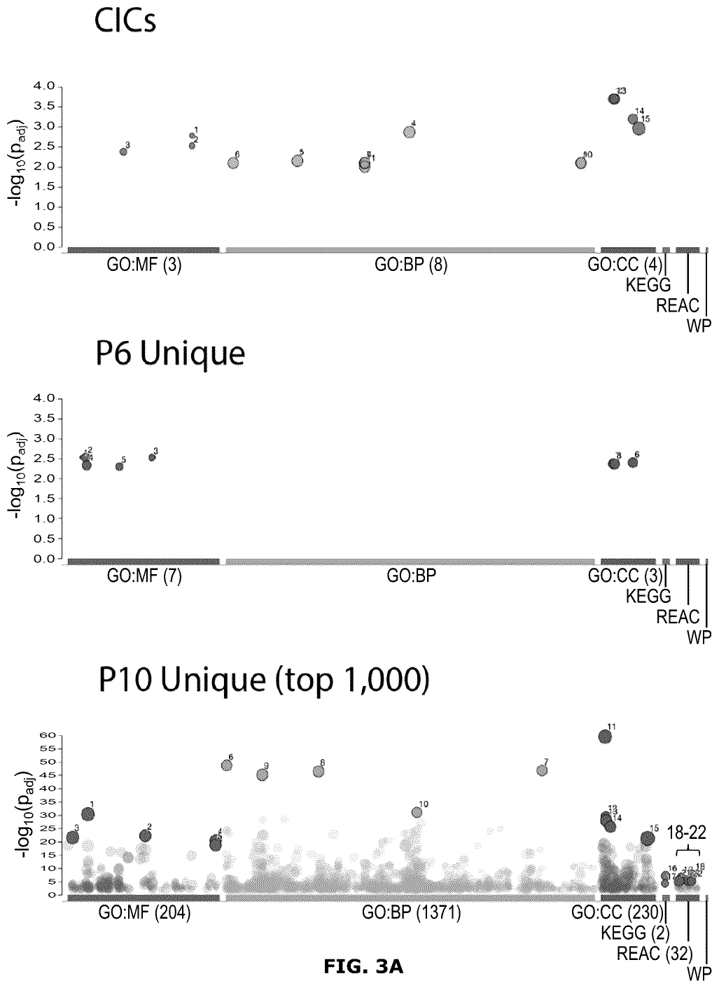

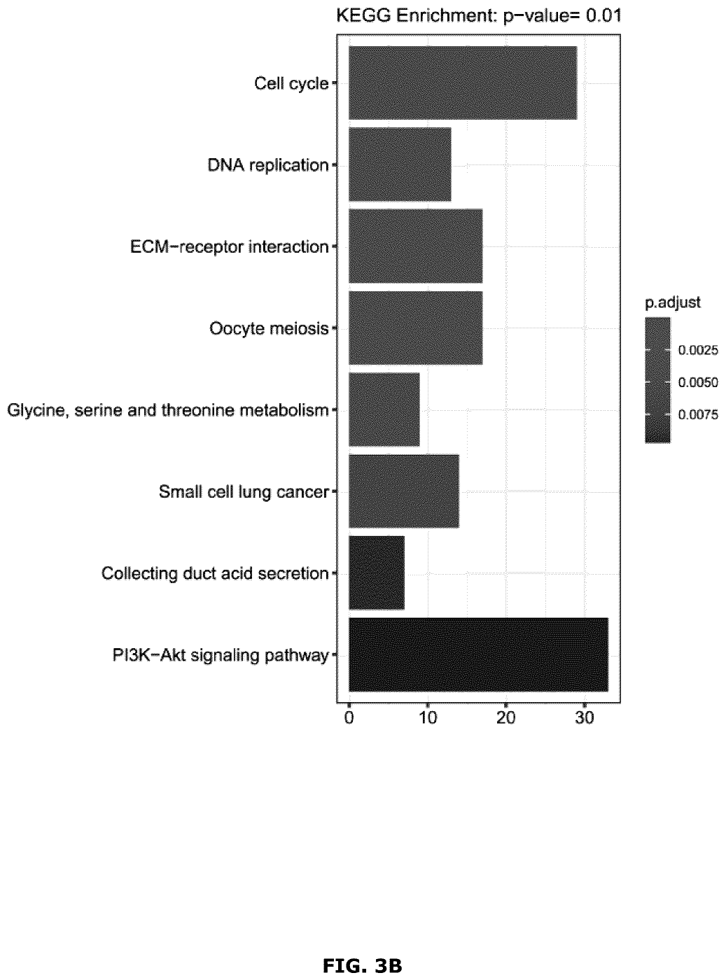

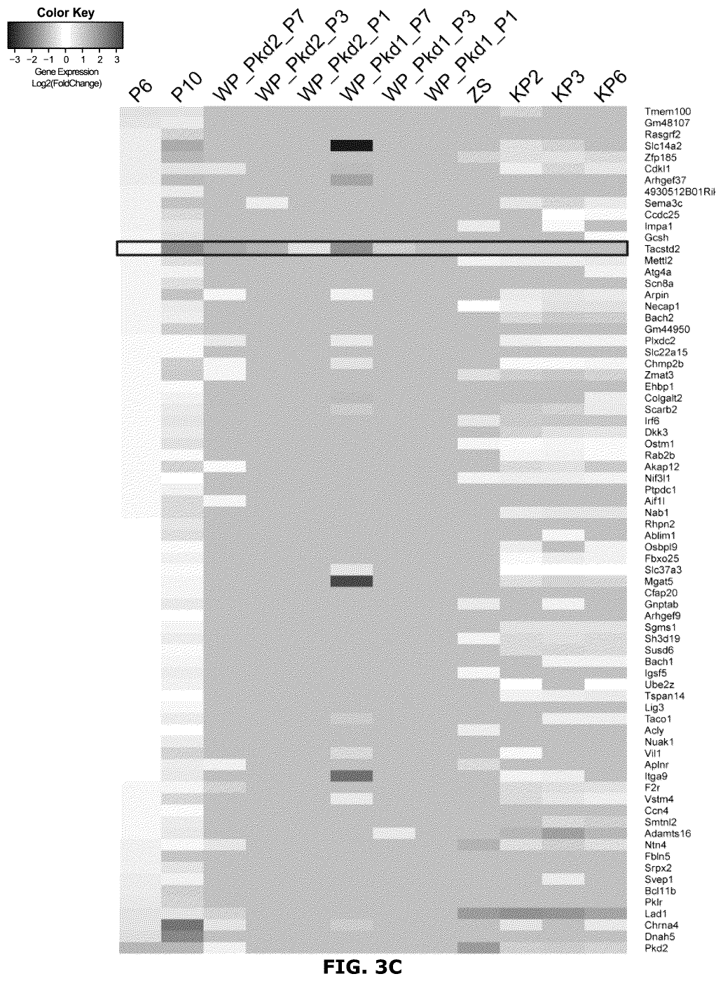

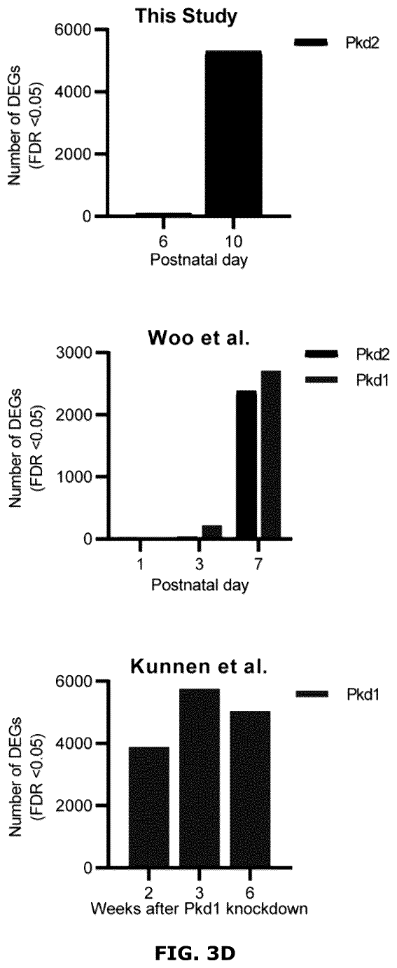

Functional enrichment analysis was used to characterize the differentially expressed genes in each category: unique to P6, common to P6 and P10 (CICs), and unique to P10 (FIGS. 3A, 3B; Tables 4-6). g:Profiler g:Gost (63) was used to find enrichment of a ranked gene list in multiple databases simultaneously. The analysis included Gene Ontology (GO) molecular function (MF), biological process (BP) and cellular component (CC) as well as Kyoto Encyclopedia of Genes and Genomes (KEGG) and Reactome (REAC) and WikiPathways (WP). The CICs were enriched in functions including integrin binding involved in cell-matrix adhesion (Svep1, Itga9), cell motility (Svep1, Arpin, Sema3c, Srpx2, Itga9, Akap12, Tacstd2, Bcl11b, Mgat5, Cfap20, Ccdc25, Vil1, Vstm4, Ccn4, Ntn4, Dnah5), and extracellular matrix (Fbln5, Svep1, Sema3c, Srpx2, Plxdc2, Lad1). The 17 genes unique to P6 were enriched in GO terms related to extracellular matrix formation (Fbn1, Col14a1, Col6a3, Sparcl1) and lipid transport (Spns2, Plscr4, Mttp). The top 1000 DEGs unique to P10 were analyzed and corresponded to broad GO terms and pathways involved in cellular proliferation including protein binding, cell cycle, cytoskeleton, and DNA replication. These results suggested that Pkd2 loss leads to early changes in cell motility programs, involving extracellular matrix. However, most CICs were not enriched in established functions, prompting a search for alternative methods of characterizing these genes.

TABLE 4
Gene ontology terms enriched among cyst initiation
candidates (CICs) common to P6 and P10.

| ID | Source | Term ID | Term Name | p_{adj} |
|---|---|---|---|---|
| 1 | GO: MF | GO: 0098640 | integrin binding involved in cell-matrix adhesion | 1.66E−03 |
| 2 | GO: MF | GO: 0098634 | cell-matrix adhesion mediator activity | 2.98E−03 |
| 3 | GO: MF | GO: 0031420 | alkali metal ion binding | 4.21E−03 |
| 4 | GO: BP | GO: 0048870 | cell motility | 1.37E−03 |
| 5 | GO: BP | GO: 0016477 | cell migration | 7.13E−03 |
| 6 | GO: BP | GO: 0001944 | vasculature development | 8.05E−03 |
| 7 | GO: BP | GO: 0040011 | locomotion | 8.05E−03 |
| 8 | GO: BP | GO: 0040017 | positive regulation of locomotion | 8.05E−03 |
| 9 | GO: BP | GO: 2000145 | regulation of cell motility | 8.05E−03 |
| 10 | GO: BP | GO: 2000147 | positive regulation of cell motility | 8.05E−03 |
| 11 | GO: BP | GO: 0040012 | regulation of locomotion | 9.98E−03 |
| 12 | GO: CC | GO: 0030312 | external encapsulating structure | 2.01E−04 |
| 13 | GO: CC | GO: 0031012 | extracellular matrix | 2.01E−04 |
| 14 | GO: CC | GO: 0062023 | collagen-containing extracellular matrix | 6.40E−04 |
| 15 | GO: CC | GO: 0071944 | cell periphery | 1.11E−03 |

TABLE 5
Gene ontology terms enriched among cyst initiation
candidates (CICs) unique to P6.

| ID | Source | Term ID | Term Name | p_{adj} |
|---|---|---|---|---|
| 1 | GO: MF | GO: 0004753 | saccharopine dehydrogenase activity | 2.99E−03 |
| 2 | GO: MF | GO: 0005201 | extracellular matrix structural constituent | 2.99E−03 |
| 3 | GO: MF | GO: 0046624 | sphingolipid transporter activity | 2.99E−03 |
| 4 | GO: MF | GO: 0005319 | lipid transporter activity | 4.71E−03 |
| 5 | GO: MF | GO: 0030020 | extracellular matrix structural constituent | 5.07E−03 |
| | | | conferring tensile strength | |
| 6 | GO: CC | GO: 0062023 | collagen-containing extracellular matrix | 4.04E−03 |
| 7 | GO: CC | GO: 0030312 | external encapsulating structure | 4.31E−03 |
| 8 | GO: CC | GO: 0031012 | extracellular matrix | 4.31E−03 |

TABLE 6
Gene ontology terms enriched among cyst initiation
candidates (CICs) unique to P10.

| ID | Source | Term ID | Term Name | p_{adj} |
|---|---|---|---|---|
| 1 | GO: MF | GO: 0005515 | protein binding | 5.04E−31 |
| 2 | GO: MF | GO: 0043168 | anion binding | 6.53E−23 |
| 3 | GO: MF | GO: 0003824 | catalytic activity | 2.93E−22 |
| 4 | GO: MF | GO: 1901363 | heterocyclic compound binding | 4.10E−21 |
| 5 | GO: MF | GO: 1901265 | nucleoside phosphate binding | 2.30E−19 |
| 6 | GO: BP | GO: 0000278 | mitotic cell cycle | 2.67E−49 |
| 7 | GO: BP | GO: 1903047 | mitotic cell cycle process | 1.95E−47 |
| 8 | GO: BP | GO: 0022402 | cell cycle process | 4.54E−47 |
| 9 | GO: BP | GO: 0007049 | cell cycle | 8.04E−46 |
| 10 | GO: BP | GO: 0051301 | cell division | 1.13E−31 |
| 11 | GO: CC | GO: 0005737 | cytoplasm | 3.73E−60 |
| 12 | GO: CC | GO: 0005819 | spindle | 3.74E−30 |
| 13 | GO: CC | GO: 0005856 | cytoskeleton | 1.48E−28 |
| 14 | GO: CC | GO: 0015630 | microtubule cytoskeleton | 3.15E−26 |
| 15 | GO: CC | GO: 0110165 | cellular anatomical entity | 6.39E−22 |
| 16 | KEGG | KEGG: 04110 | Cell cycle | 8.47E−08 |
| 17 | KEGG | KEGG: 03030 | DNA replication | 5.61E−05 |
| 18 | REAC | REAC: R-MMU-2500257 | Resolution of Sister | 1.67E−08 |
| | | | Chromatid Cohesion | |
| 19 | REAC | REAC: R-MMU-9648025 | EML4 and NUDC in mitotic | 2.25E−07 |
| | | | spindle formation | |
| 20 | REAC | REAC: R-MMU-68877 | Mitotic Prometaphase | 3.48E−06 |
| 21 | REAC | REAC: R-MMU-69278 | Cell Cycle, Mitotic | 6.62E−06 |
| 22 | REAC | REAC: R-MMU-5663220 | RHO GTPases Activate Formins | 6.89E−06 |

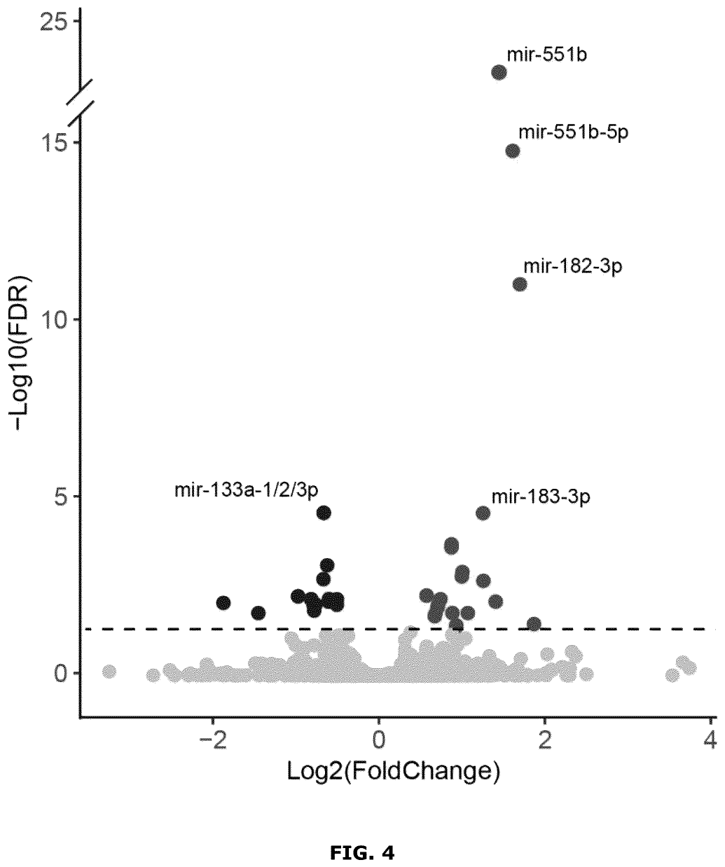

There were no differentially expressed miRNAs in the P6 dataset that reached significance (FDR<0.05). At this early pre-cystic time point, any differences in miRNA expression were likely subtle and below the level of detection by bulk sequencing. Nevertheless, 39 stem-loop and mature miRNAs with FDR<0.05 were found at P10 (FIG. 4). Twenty-four of the 39 genes were previously cited in PKD literature (Table 7), further validating the ability of the research described herein to detect relevant changes in small RNA expression between experimental and control kidneys. The most significantly elevated miRNA, mir-551b, is implicated in abnormal cell differentiation and proliferation in gastric cancer (12) and ovarian cancer (13). Mir-551b may warrant further study in relation to PKD pathogenesis. The remainder of this study focuses on mRNAs with differential expression in pre-cystic kidneys as novel candidates for cyst initiation.

TABLE 7
Postnatal day 10 differentially expressed microRNAs
(FDR < 0.05) associated with PKD in cited literature.

| | | | Mir Gene Family | |
|---|---|---|---|---|
| miRBase ID* | log₂FC | FDR | or Cluster | Literature |
| mmu-miR-18a-3p | 0.889 | 0.018 | 17-92 cluster | Patel et al. (2013) |
| mmu-miR-92b-3p | 0.710 | 0.012 | 17-92 cluster | Patel et al. (2013) |
| mmu-miR-92b | 0.709 | 0.012 | 17-92 cluster | Patel et al. (2013) |
| mmu-miR-182-3p | 1.702 | 0.000 | 182 cluster | Pandey et al. (2011) |
| | | | | Woo et al. (2017) |
| mmu-miR-183-3p | 1.258 | 0.000 | 182 cluster | Pandey et al. (2011) |
| | | | | Woo et al. (2017) |
| mmu-miR-182 | 1.010 | 0.001 | 182 cluster | Pandey et al. (2011) |
| | | | | Woo et al. (2017) |
| mmu-miR-182-5p | 1.002 | 0.002 | 182 cluster | Pandey et al. (2011) |
| | | | | Woo et al. (2017) |
| mmu-miR-183 | 0.745 | 0.007 | 182 cluster | Pandey et al. (2011) |
| | | | | Woo et al. (2017) |
| mmu-miR-183-5p | 0.717 | 0.010 | 182 cluster | Pandey et al. (2011) |
| | | | | Woo et al. (2017) |
| mmu-miR-96 | 0.683 | 0.019 | 182 cluster | Pandey et al. (2011) |
| | | | | Woo et al. (2017) |
| mmu-miR-96-5p | 0.677 | 0.022 | 182 cluster | Pandey et al. (2011) |
| | | | | Woo et al. (2017) |
| mmu-miR-21a | 0.935 | 0.039 | 21 family | Lakhia et al. (2016) |
| mmu-miR-21a-5p | 0.934 | 0.043 | 21 family | Lakhia et al. (2016) |
| mmu-miR-222-3p | 0.577 | 0.006 | 221 cluster | Ben-Dov et al. (2014) |
| mmu-miR-222 | 0.577 | 0.006 | 221 cluster | Ben-Dov et al. (2014) |
| mmu-miR-551b-5p | 1.615 | 0.000 | 551 family | Ben-Dov et al. (2014) |
| mmu-miR-551b | 1.445 | 0.000 | 551 family | Ben-Dov et al. (2014) |
| mmu-miR-133a-3p | −0.662 | 0.000 | 133 family | Ben-Dov et al. (2014) |
| mmu-miR-133a-1 | −0.662 | 0.000 | 133 family | Ben-Dov et al. (2014) |
| mmu-miR-133a-2 | −0.662 | 0.000 | 133 family | Ben-Dov et al. (2014) |
| mmu-miR-338 | −0.620 | 0.001 | 338 family | Ben-Dov et al. (2014) |
| mmu-miR-338-5p | −0.665 | 0.002 | 338 family | Ben-Dov et al. (2014) |
| mmu-miR-488 | −0.813 | 0.007 | 488 family | Pandey et al. (2011) |
| mmu-miR-488-3p | −0.813 | 0.007 | 488 family | Pandey et al. (2011) |

*miRBase Release 22.1

### Example 2

### Exploring Cyst Initiation Candidates in Published RNAseq Datasets

### Materials and Methods

### Comparison to Published Bulk RNAseq Data

Comparisons to published mouse datasets were performed by filtering publicly available data for genes with false discovery rate (FDR)<0.05 and comparing them against genes in the currently disclosed dataset with FDR<0.05. Simple linear regression analysis was performed using GraphPad Prism 9, Version 9.5.1 (GraphPad Software, Boston, MA). Datasets include:
a) Kunnen et al. (22) Supplementary Table S9. Fold change of differentially expressed genes in mice deleted of Pkd1 by KspCad-Cre^{ERT2} compared to Pkd1 wild type controls.
b) Woo et al. (14) Supplementary Table S2. Differentially expressed genes in mice deleted of Pkd1 by HoxB7-Cre.
c) Woo et al. (14) Supplementary Table S3. Differentially expressed genes in mice deleted of Pkd2 by HoxB7-Cre.
d) Zhang et al. (23) Supplementary Data 2. Differentially expressed genes in mice deleted of Pkd2 by Pax8^{rtTA}:TetO-Cre.

### Results

To further identify the cyst initiation candidates (CICs) most likely to drive early cystic transformation, existing data from the field was leveraged, with reasoning that strong cyst initiators would be consistent in alternative mouse models of early PKD (see Table 8 and Kunnen et al. (22), Zhang et al. (23), and Woo et al. (14)). Therefore, data disclosed herein was compared to models differing in Cre expression, target gene (Pkd2 vs. Pkd1), age and method of induction, age at sacrifice, cyst burden at time of collection, and number of mice used for analysis (Table 8).

TABLE 8
Early and pre-cystic mouse models with RNAseq data

| | Cre Strain | Target | | Age at | Degree |
|---|---|---|---|---|---|
| Publication | Expression | Gene | Induction conditions | sacrifice | of Cysts |
| This Work | Cagg-Cre^{ER} | Pkd2 | Tamoxifen IP at P2 | P6 | Pre |
| | whole body | | | | |
| | Cagg-Cre^{ER} | Pkd2 | Tamoxifen IP at P2 | P10 | Early |
| | whole body | | | | |
| Kunnen | KspCad-Cre^{ERT2} | Pkd1 | Tamoxifen in food 5 mg/day × | 13/14 + | Pre |
| et al. | kidney specific | | 3 days at 13-14 weeks of | 2 weeks | |
| (22) | | | age | | |
| | KspCad-Cre^{ERT2} | Pkd1 | Tamoxifen in food 5 mg/day × | 13/14 + | Pre |
| | kidney specific | | 3 days at 13-14 weeks of | 3 weeks | |
| | | | age | | |
| | KspCad-Cre^{ERT2} | Pkd1 | Tamoxifen in food 5 mg/day × | 13/14 + | Pre |
| | kidney specific | | 3 days at 13-14 weeks of | 6 weeks | |
| | | | age | | |
| Zhang | Pax8^{rtTA}, TetO-Cre | Pkd2 | Doxycycline in drinking | P70 | Pre |
| et al. | kidney selective, | | water + sucrose 2 mg/mL for | | |
| (23) | whole nephron | | 2 weeks starting at P28-P42 | | |
| Woo | HoxB7-Cre | Pkd2 | HoxB7-Cre becomes active | P1 | Pre |
| et al. | collecting duct | | E9.5-E12.5 | | |
| (14) | HoxB7-Cre | Pkd2 | HoxB7-Cre becomes active | P3 | Early |
| | collecting duct | | E9.5-E12.5 | | |
| | HoxB7-Cre | Pkd2 | HoxB7-Cre becomes active | P7 | Mid |
| | collecting duct | | E9.5-E12.5 | | |
| | HoxB7-Cre | Pkd1 | HoxB7-Cre becomes active | P1 | Pre |
| | collecting duct | | E9.5-E12.5 | | |
| | HoxB7-Cre | Pkd1 | HoxB7-Cre becomes active | P3 | Early |
| | collecting duct | | E9.5-E12.5 | | |
| | HoxB7-Cre | Pkd1 | HoxB7-Cre becomes active | P7 | Mid |
| | collecting duct | | E9.5-E12.5 | | |

Woo et al. (14) looked at disease progression in young mice at P1, P3, and P7 where Pkd1 or Pkd2 had been deleted during development of the collecting duct with HoxB7-Cre. This study observed few significant transcriptomic changes at the earliest time points after polycystin inactivation and the changes observed did not correlate strongly with DEGs identified in the present disclosure (FIGS. 3C, 3D). However, two other studies looking at gene expression in pre-cystic adult mouse models correlated significantly with data at P6 and P10 (FIGS. 2E, 3C, 3D). Kunnen et al. deleted Pkd1 in adult mouse kidneys via Ksp-Cre (22) and Zhang et al. deleted Pkd2 in collecting ducts and distal tubules of mature adult mice with doxycycline-inducible Pax8^{rtTA}, TetO-Cre (23). Remarkably, overall agreement was observed in pre-cystic kidneys even between Pkd2 and Pkd1 models and between juvenile and mature mice.

### Example 3

### Cellular Origins of Cyst Initiation Candidates

### Materials and Methods

### Comparison to Published Single-Cell RNAseq Analysis

### Gene Expression Omnibus (GEO) Murine

The work presented herein examines changes in whole-kidney gene expression following Pkd2 deletion but does not identify which of the diverse kidney cell types express the gene. To determine whether there is any cell-specific localization of candidate genes, data from Ransick et al. (16), who identified enriched transcriptome profiles within individual renal cell types, was reanalyzed. Murine transcriptomes were obtained as 12 objects from GSE129798 (16). Raw data was filtered to retain cells that expressed between 200 and 6,000 unique genes and a mitochondrial fraction less than 10%. A total of 31,056 cells from the cortex, outer medulla, and inner medulla fit these criteria. Each dataset was further pre-processed by log normalizing each dataset with a scale factor of 10,000 and finding 2,000 variable features using the default ‘vst’ method in seurat3.0 (48). The dataset was then scaled to regress out differences driven by unequal total counts captured and mitochondrial capture. The top 40 principal components were used for construction of a Uniform Manifold Approximation and Projection (UMAP) and for neighbor finding. During clustering, a resolution parameter of 0.5 was utilized. The resulting 28 Louvain clusters were visualized in 2D and/or 3D space and were annotated using known biological cell-type markers (17).

### GEO Wild-Type and ADPKD Human

To determine if candidate cyst initiation genes from the mouse Pkd2 model were also expressed in human kidneys, published kidney transcriptome data from human PKD patients and from healthy controls was reanalyzed. Data were obtained as 13 objects from GSE185948 (19). The data was filtered to only retain cells that expressed between 500 and 6,000 unique genes and a mitochondrial fraction less than 10%. Heterotypic doublets were identified with DoubetFinder v2 assuming 8% of cells represented heterotypic doublets. The datasets were then integrated in Seruat using “IntegrateData”. These parameters resulted in retention of 56,161 cells from the healthy controls and 69,385 cells from PKD patients. Each dataset was further pre-processed by log normalizing each dataset with a scale factor of 10,000 and finding 2,000 variable features using the default ‘vst’ method in seurat3.0 (48). The top 50 principal components were used for initial integration. The dataset was then scaled to regress out differences driven by unequal total counts captured and mitochondrial capture. The ADPKD and Healthy control datasets were then integrated with batch effect correction using Harmonyv1. The top 15 principal components were used for construction of a UMAP and for neighbor finding. During clustering, a resolution parameter of 0.5 was utilized. The resulting 18 Louvain clusters were visualized in 2D and/or 3D space and were annotated using known biological cell-type markers (17).

For pseudobulk analysis, cells were grouped by their disease state, and the Seurat function “AverageExpression” was used to average expression across all cell types present in each sample. This data was then rescaled and visualized in heatmaps. Differentially expressed genes between the ADPKD and healthy controls were calculated using “FindMarkers” filtering for transcripts with a log 2FC greater than 0.25 and expression in greater than 20% of cells.

### Results

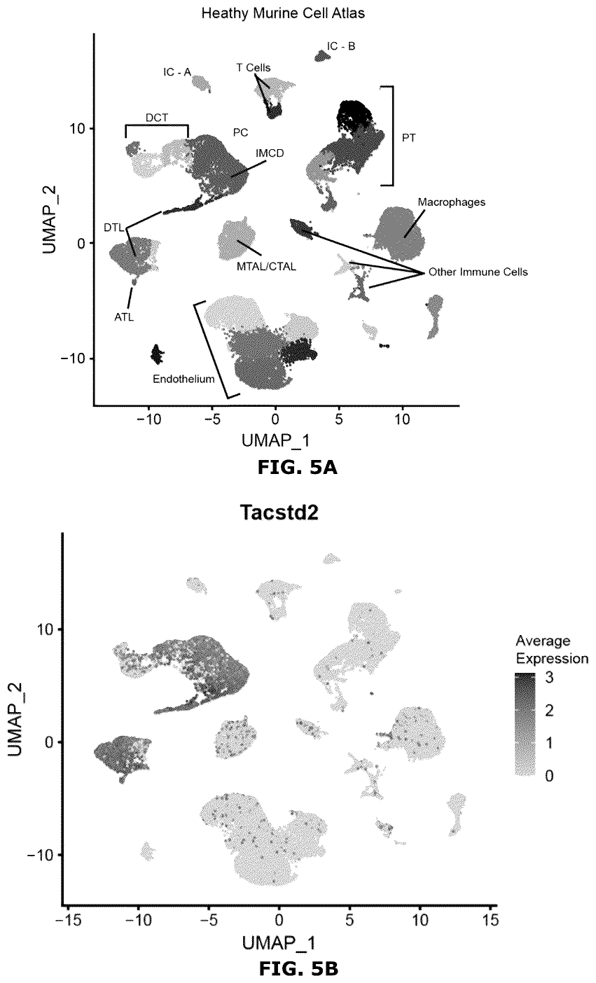

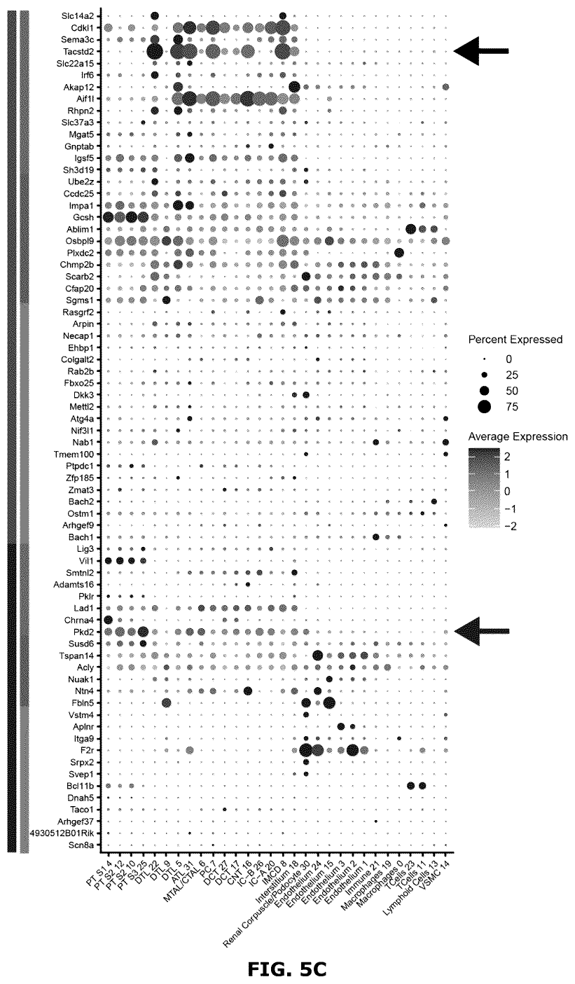

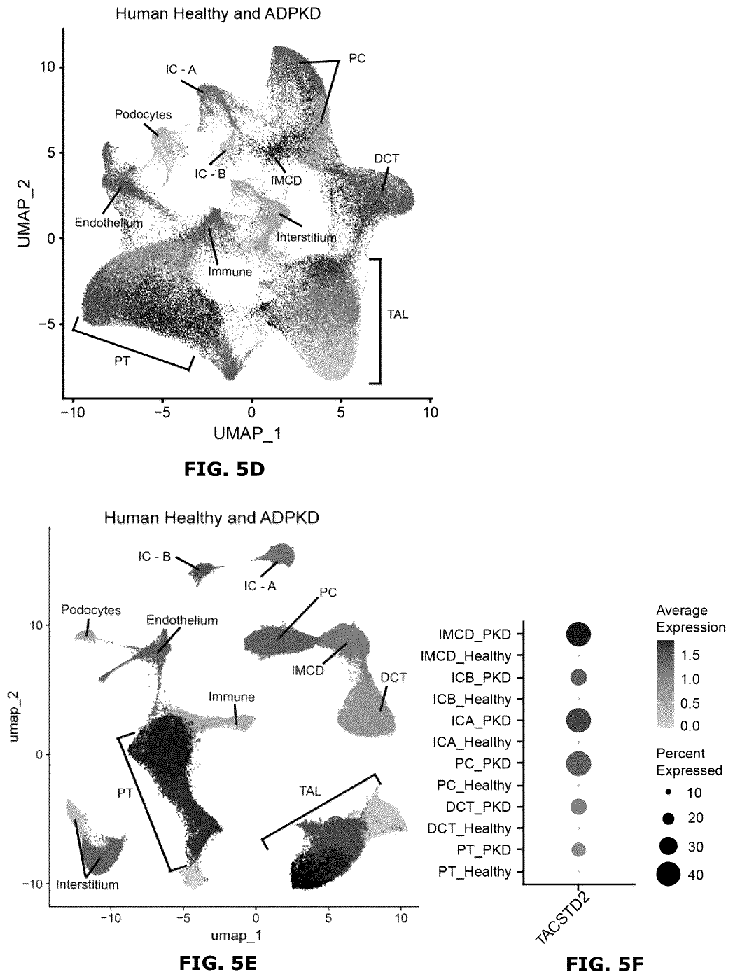

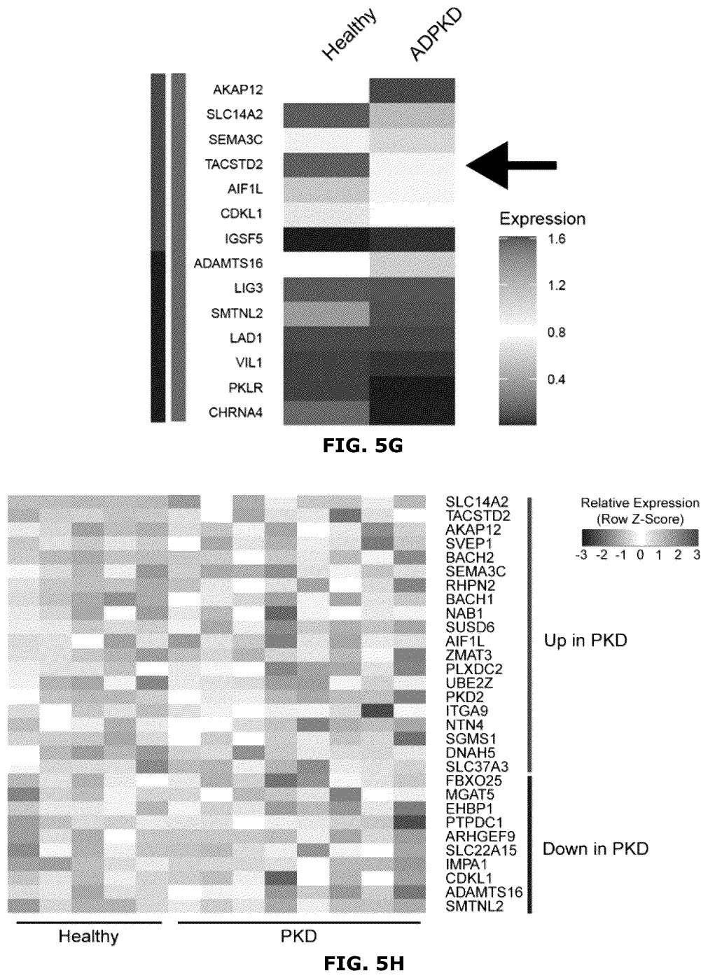

To focus on the genes most relevant to human disease, CICs with conserved expression patterns were identified by reanalyzing Muto et al.'s (19) single cell data derived from healthy and ADPKD human kidneys. The Muto et al. data was re-calculated and cell types assigned according to consensus markers from Chen et al. (FIGS. 5D, 5E). To compare transcriptional differences present in human disease, Muto et al.'s data was first pseudo bulked to identify differentially expressed genes and then compared these genes to the disclosed list of 74 CICs (FIGS. 5G, 5H). Thirty DEGs were found in common between the human data and the 74 CICs. The majority agreed in direction between human disease and the disclosed early cystic murine model. Others (such as Cdkl1) demonstrated differential expressions in directions counter to disclosed observations (FIGS. 5G, 5H). Genes with discrepant expression remain of interest, as currently available human data necessarily derives from later disease stages than mouse studies, and thus may not fully constitute the milieu of cyst initiation.

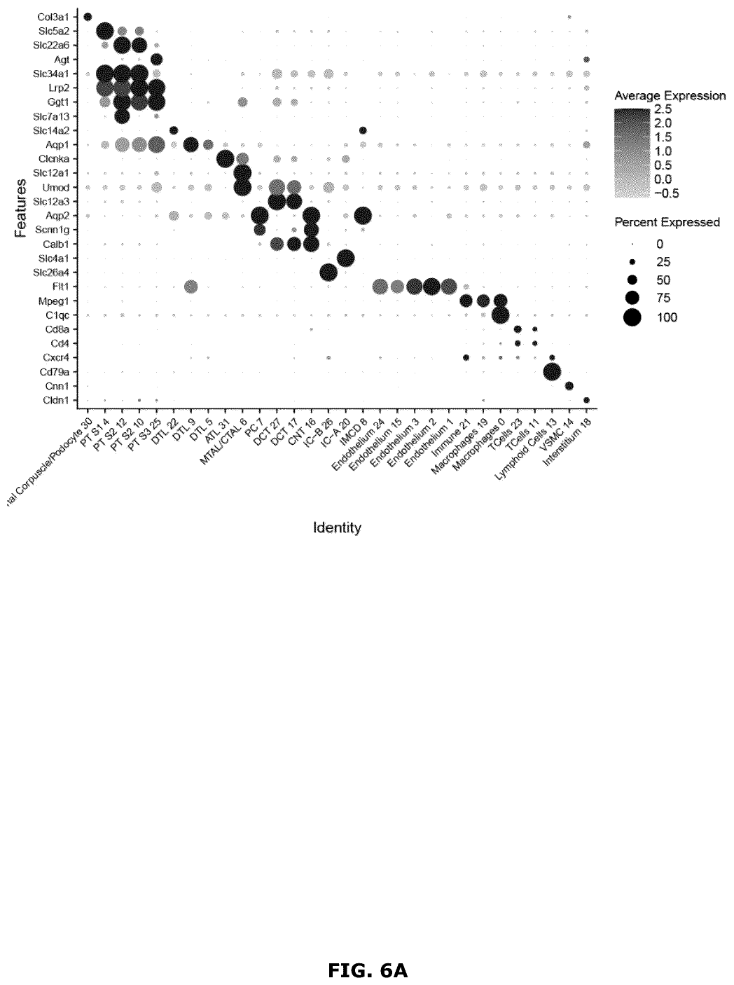

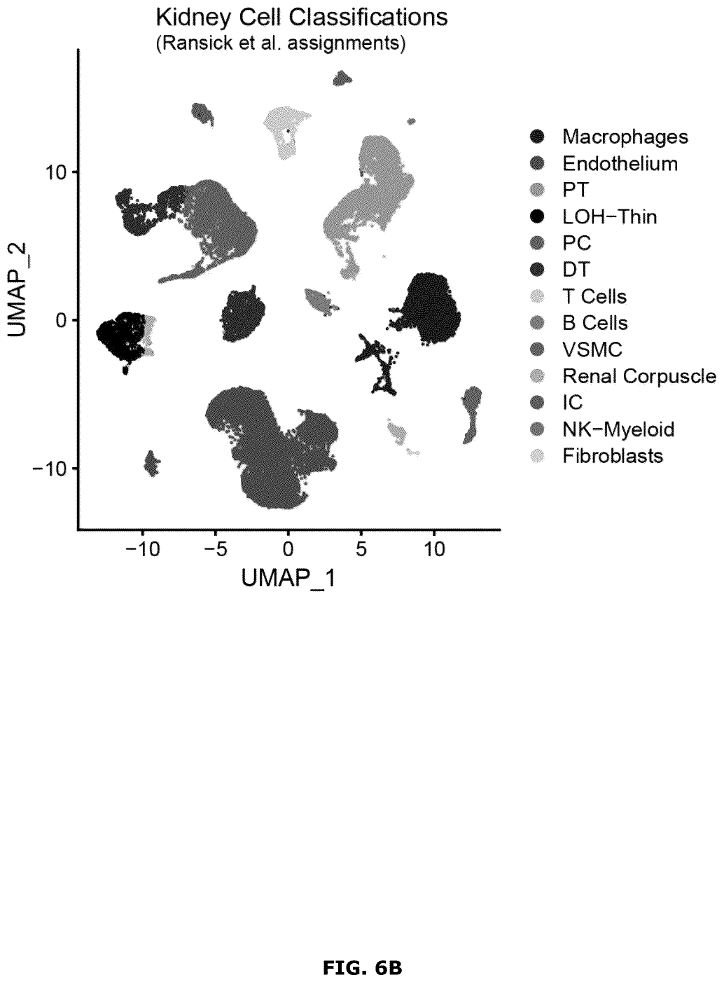

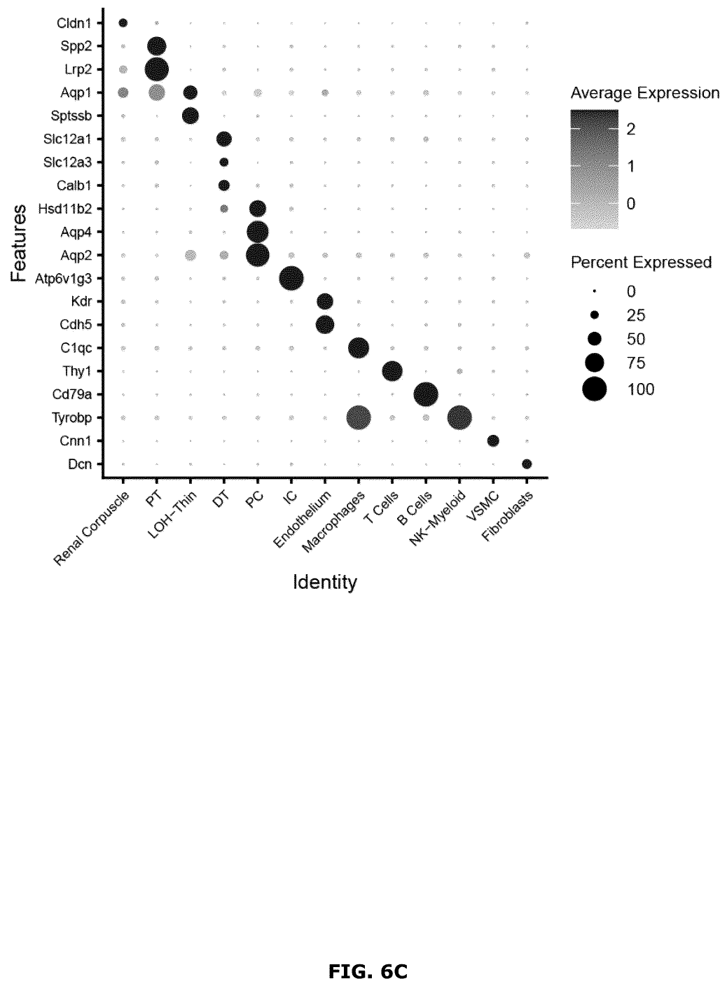

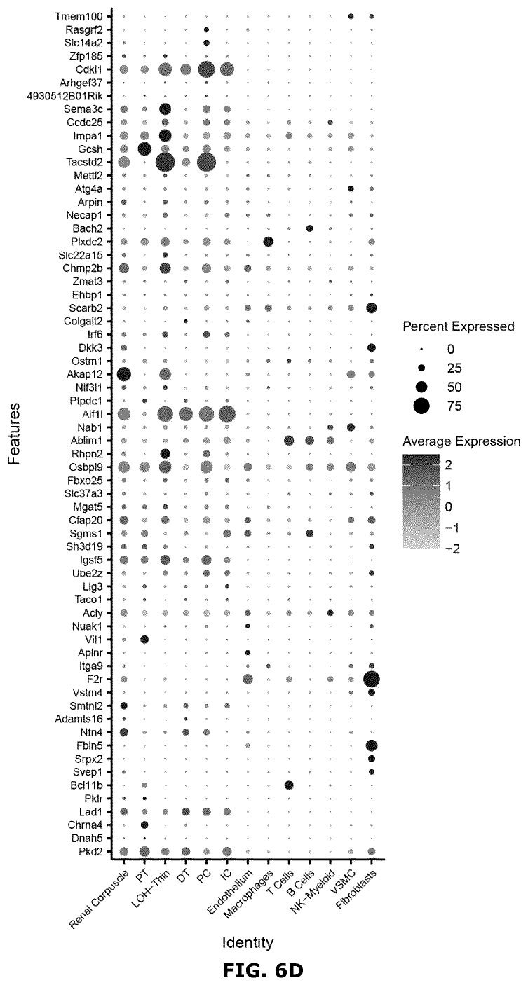

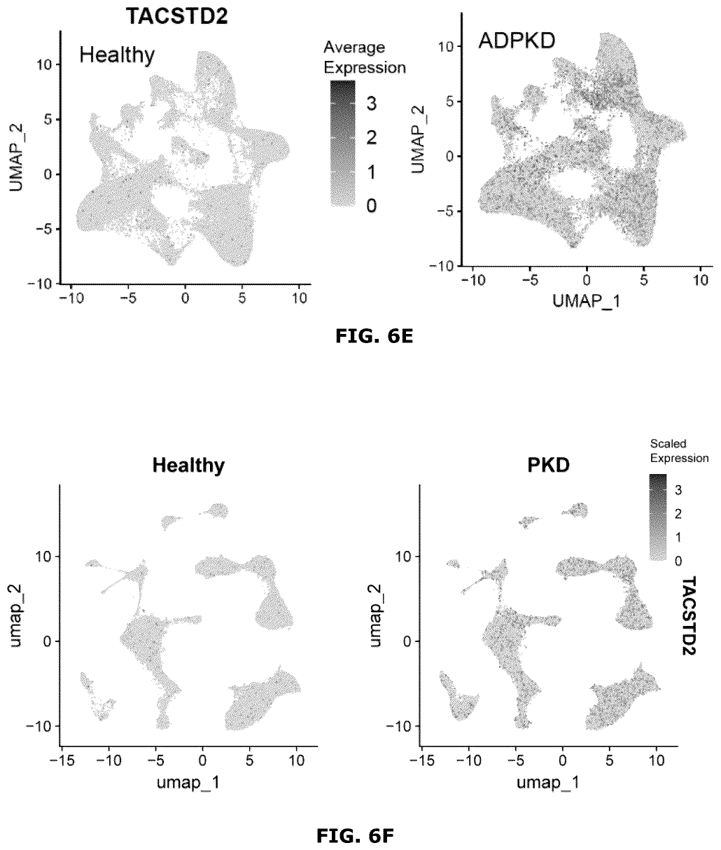

Factors that drive initial cyst growth are likely to be expressed in epithelial cells as these are sites of the first pathology in cystic disease (15). Single cell sequence data from mouse kidney has more clearly defined cell types than equivalent human data allowing for better assignment of genes to cell types. Thus to identify the particular cell types where CICs were expressed, published single cell transcriptomes from healthy mouse kidneys (16) were reanalyzed to construct a uniform manifold projection (FIG. 5A) where the identities of cell clusters were assigned using consensus marker genes from Chen et al. (17) (FIG. 6B). The clusters broadly agreed with original cell type assignments by Ransick et al. (16) (FIGS. 6A-6C). The three main expression patterns found include genes restricted to epithelial cells (genes such as Tacstd2, Cdkl1, Akap12, and Aif1L), those with broad expression in many cells (Osbpl9), and those expressed in stromal cells (Endothelium: F2R; T-cell: Bcl11B) (FIGS. 5C, 6D).

When inspecting CICs expressed in epithelial cells and showing differential expression in human PKD, the tumor associated calcium signal transducer Tacstd2 stood out. This gene, whose expression was restricted to epithelial cells of the distal tubule (FIGS. 5C, 5B) was one of the most dysregulated genes in human PKD (FDR<1E⁻¹⁰) (FIGS. 5G, 5H; Table 9). In the disclosed data, Tacstd2 was dysregulated in P6 pre-cystic kidneys, P10 early cystic kidneys, and in early time points of Woo et al.'s Pkd1 and Pkd2 models (FIG. 3C). In healthy human kidneys, most cells show low to undetectable expression, but the expression goes up considerably in the distal parts of the uriniferous tubule in PKD tissues (FIGS. 5F, 6E, 6F).

TABLE 9
DEGs common between human data and CICs.

| Gene | Avg. log₂FC | P_{adj} |
|---|---|---|
| SLC14A2 | 3.07E+00 | 0.00E+00 |
| TACSTD2 | 2.47E+00 | 0.00E+00 |
| AKAP12 | 2.09E+00 | 0.00E+00 |
| SVEP1 | 1.68E+00 | 0.00E+00 |
| BACH2 | 1.33E+00 | 0.00E+00 |
| SEMA3C | 9.26E−01 | 0.00E+00 |
| RHPN2 | 9.19E−01 | 0.00E+00 |
| BACH1 | 6.16E−01 | 0.00E+00 |
| NAB1 | 6.10E−01 | 1.31E−90 |
| SUSD6 | 6.09E−01 | 4.97E−133 |
| AIF1L | 6.06E−01 | 3.78E−276 |
| ZMAT3 | 5.91E−01 | 2.79E−82 |
| PLXDC2 | 5.07E−01 | 0.00E+00 |
| UBE2Z | 4.65E−01 | 8.06E−48 |
| PKD2 | 4.39E−01 | 1.14E−243 |
| ITGA9 | 3.93E−01 | 7.68E−26 |
| NTN4 | 3.72E−01 | 1.14E−17 |
| SGMS1 | 3.66E−01 | 3.10E−226 |
| DNAH5 | 3.37E−01 | 1.53E−215 |
| SLC37A3 | 3.09E−01 | 1.32E−06 |
| FBXO25 | −3.42E−01 | 1.70E−157 |
| MGAT5 | −3.45E−01 | 4.93E−160 |
| EHBP1 | −3.59E−01 | 0.00E+00 |
| PTPDC1 | −4.03E−01 | 3.50E−233 |
| ARHGEF9 | −4.35E−01 | 3.90E−252 |
| SLC22A15 | −5.77E−01 | 0.00E+00 |
| IMPA1 | −5.95E−01 | 0.00E+00 |
| CDKL1 | −6.63E−01 | 0.00E+00 |
| ADAMTS16 | −8.49E−01 | 0.00E+00 |
| SMTNL2 | −1.59E+00 | 0.00E+00 |

To understand how TACSTD2 expression differs in humans with ADPKD, single cell data derived from healthy and ADPKD human kidneys from Muto et al. (19) was reanalyzed. To have consistency with the mouse single cell data, the Muto et al. data was re-calculated and cell types assigned according to consensus markers from Chen et al. (FIGS. 5D, 5E). In healthy kidneys, TACSTD2 exhibited trace expression throughout the kidney. However, in adult polycystic kidneys, TACSTD2 was robustly expressed in a multitude of cells, including principal and inner medullary cells of the collecting duct, where high expression also was observed in mouse (FIGS. 5G, 5H, 6E, 6F). Some CICs (such as CDKL1) demonstrated differential expression in directions counter to previous observations (FIG. 5G, 5H). These genes may still represent initiator events, as end stage ADPKD represents a vastly different environment than cyst initiation. Nevertheless, differences between mouse and human cell type expression are a consideration for evaluating candidates for therapeutic design.

### Example 4

### Characterizing Tacstd2 as a Cyst Initiation Candidate

### Materials and Methods

### Kidney Organoid Differentiation

Human induced pluripotent stem (IPS) cell lines, UAB-iPSC Parental-Normal Karyotype, UAB-iPSC-PKD1 KO #1 (PKD1^{-/-}), and UAB-iPSC-PKD2 KO #1 (PKD2^{-/-}) were obtained from PKD Research Resource Consortium University of Maryland (Baltimore, MD) and verified to be of human origin and to have the correct genotype (wild type, PKD1^{-/-}, or PKD2^{-/-}) by sequencing of the regions of PKD1 and PKD2 (see table below; sequences provided by University of Alabama PKD Center; primers manufactured by Genewiz).

| Target | Primer | 5′ Sequence | SEQ ID NO. |
|---|---|---|---|
| PKD1 | Forward | CCGGAGGCAACAGTGGAGCATGT | 1 |
| | Reverse | GCACGGGTGGGGGCGGCTTCCTC | 2 |
| PKD2 | Forward | GCCAATTTTGTTTCCCATAAGCC | 3 |
| | Reverse | GCAAAGCAGGTCCTGTCGAT | 4 |

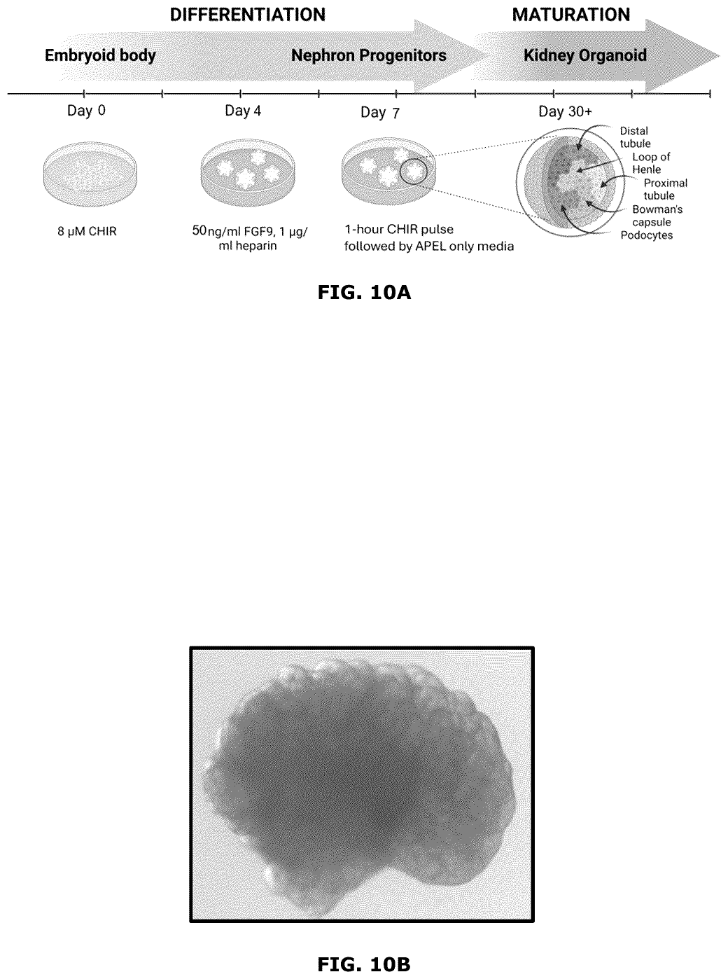

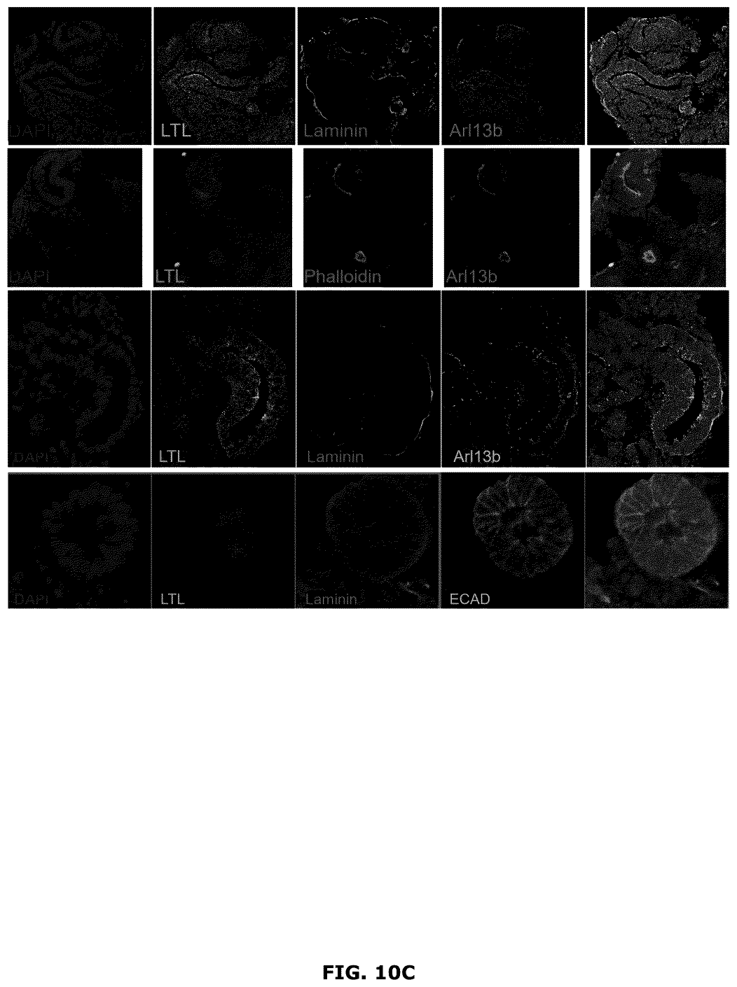

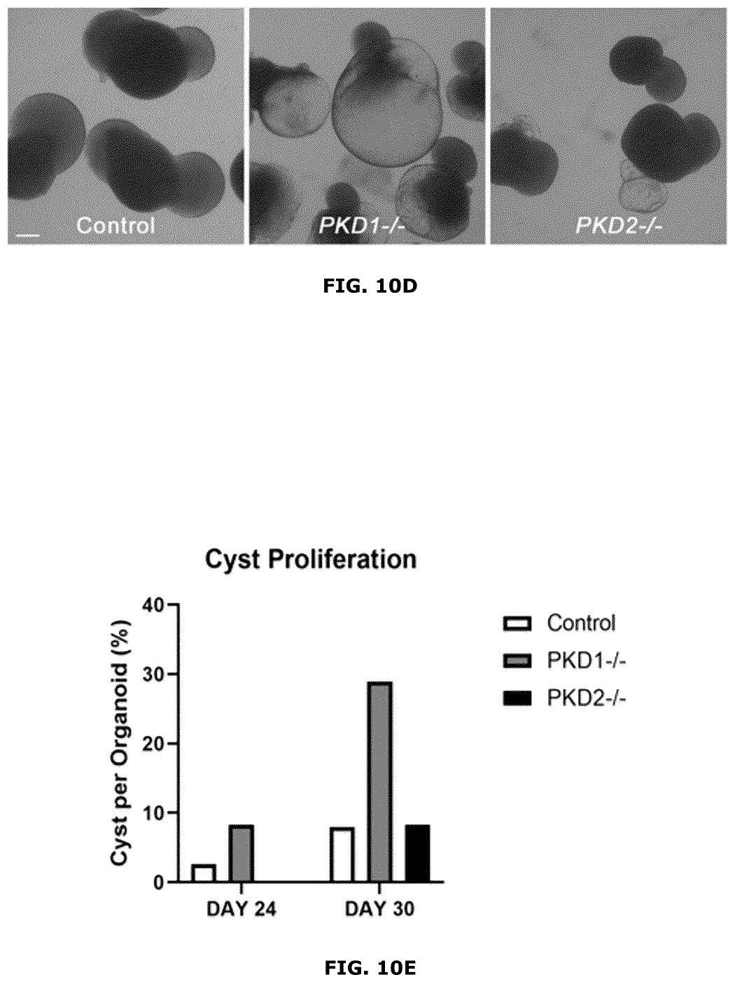

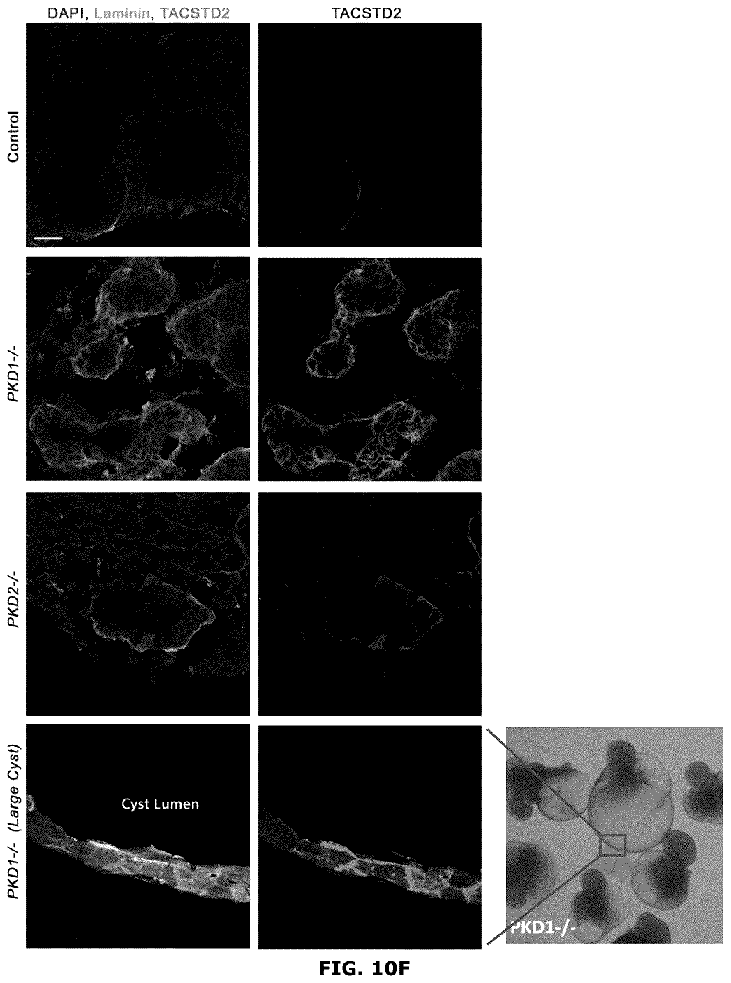

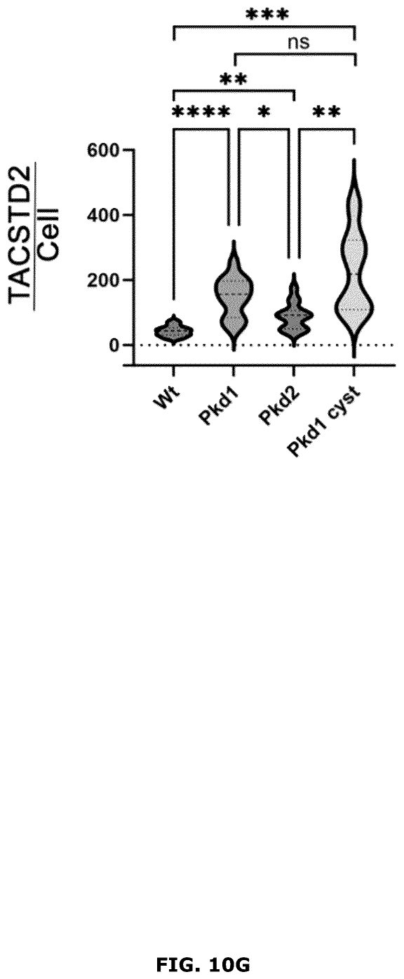

To produce organoids, IPS cells were plated at 7,300 cells per cm2 in mTeSR™1 medium (STEMCELL Technologies, Vancouver, Canada; Catalog No. 85850) on 6-well plates coated with Gibco™ vitronectin (Thermo Fisher Scientific, Waltham, MA; Catalog No. A14700). On day 0, differentiation was initiated by replacing mTeSR™1 medium with STEMdiff™ APEL™2 medium (STEMCELL Technologies Catalog No. 05275) containing 8 μM CHIR99021 (Abcam, Cambridge, UK; Catalog No. ab120890) for four days. The medium was replaced with fresh medium every two days. On day 4, cells were washed with Gibco™ DPBS (Dulbecco's PBS), no calcium, no magnesium (Thermo Fisher Scientific Catalog No. 14190136), and medium was replaced with fresh STEMdiff™ APEL™2 medium containing 50 ng/ml of recombinant human FGF9/GAF protein (Abcam Catalog No. ab50034) and a final concentration of 1 μg/mL of 0.2% heparin sodium salt in dissolved in PBS (STEMCELL Technologies Catalog No. 07980) for three days. On day 7, the medium was replaced with STEMdiff™ APEL™2 medium containing 5 μM CHIR99021 for 1 hour. Cells were then washed and dislodged using Gibco™ TrypLE™ Express Enzyme without phenol red (Thermo Fisher Scientific Catalog No. 12604013) and pelleted. The cells were then resuspended in STEMdiff™ APEL™2 medium containing 50 ng/mL FGF9/GAF and 1 μg/mL heparin sodium salt, transferred to a CORNING® COSTAR® Ultra-Low Attachment 6-well plate (MilliporeSigma, Burlington, MA; Catalog No. CLS3471) and placed on an orbital shaker at 100 revolutions per minute (rpm) in a 37° C. 5% CO₂ incubator. Medium was changed every two days. After five days, the medium was replaced with STEMdiff™ APEL™2 without additions and changed every two days. Organoids were typically harvested 14 days later. This protocol is a modification of Ruiter et al. (21) (FIGS. 10A, 10B).

### Histology

### Mouse Tissues

For histology of P21 kidneys (FIGS. 7A-7D only), mothers nursing Pkd2^{flox/flox}:Rosa26-Cre^{ERT} pups were dosed with tamoxifen (200 mg/kg) by oral gavage on P2, postnatal day 3 (P3), and postnatal day 4 (P4). The Pkd2^{flox/flox}:Rosa26-Cre^{ERT} pups remained with nursing mothers until euthanasia at P21 as previously described (20).

Tissues were fixed by immersion overnight (about 16 hours) in 10% formalin (Electron Microscopy Sciences) in phosphate-buffered saline and then embedded in paraffin. Sections were deparaffinized and stained with hematoxylin and eosin (H&E).

Images of H&E-stained sections were obtained with a Zeiss Axio Scan.Z1 slide scanner with brightfield capabilities using the 20× objective.

For immunofluorescent staining, sections were deparaffinized, antigens were retrieved by autoclaving for 30 min in 10 mM sodium citrate, pH 6.0 and stained with primary antibodies diluted in TBST (10 mM Tris, pH 7.5; 167 mM NaCl; and 0.05% Tween 20) plus 0.1% cold water fish skin gelatin (Sigma-Aldrich). Alexa Fluor-labeled secondary antibodies (Invitrogen) were used to detect the primary antibodies. Primary antibodies used included Aquaporin-1 (1:100, Santa Cruz, sc-32737), Aquaporin-2 (1:200; Sigma Catalog No. SAB5200110), and Tacstd2/Trop-2 (F-5) (1:250, Santa Cruz Biotechnology, Dallas, TX; Catalog No. sc-376181). FITC-conjugated lectins were added with secondary antibodies: Lotus tetragonolobus agglutinin (LTA, 1:50, Vector Labs) or Dolichos biflorus agglutinin (DBA, 1:20, Vector Labs). Nuclei were labeled with 4′,6-diamidino-2-phenylindole (DAPI).

Fluorescent images were obtained with a Zeiss LSM900+ Airyscan microscope. Fluorescent slide scans were obtained using Zeiss Axio Scan.Z1 slide scanner at 20× objective.

### Human Tissues

Paraffin slides of kidney sections from 3 male ADPKD samples and 3 healthy male controls were obtained from the Polycystic Kidney Disease Research Resource Consortium at the University of Maryland (Baltimore, Maryland). No genotype or additional information is available for these specimens. Slides were deparaffinized, antigens retrieved, and stained as described above using Aquaporin-2 (1:200; Sigma Catalog No. SAB5200110), Tacstd2/Trop-2 (F-5) (1:250, Santa Cruz Catalog No. sc-376181), FITC-conjugated Lotus tetragonolobus agglutinin (LTA, 1:50, Vector Labs).

Fluorescent images were obtained with a Zeiss LSM900+ Airyscan microscope. Fluorescent slide scans were obtained using Zeiss Axio Scan.Z1 slide scanner at 20× objective.

### Organoids

Plates were tilted until kidney organoids settled to the bottom and medium was slowly removed with a 5 mL pipette. Organoids were washed with 2 mL of phosphate-buffered saline (PBS) and again allowed to settle to the bottom of the dish. After removal of the PBS, 2 mL of 4% paraformaldehyde in PBS was added for 1 hour. Following this, paraformaldehyde was removed and organoids were resuspended in 500 μL of PBS in a microcentrifuge tube. The PBS was replaced with 200 μL of 30% sucrose in 100 mM Sorensen's Phosphate Buffer. After 24 hours, the organoids were transferred to a cryomold and embedded in Tissue Plus O.C.T. Compound (Fisher HealthCare Catalog No. 4585). Embedded organoids were frozen on dry ice. Cryoblocks were sectioned at 10 microns and collected on Superfrost Plus Microscope slides (Fisherbrand Catalog No. 12-550-150).

Slides were washed with PBS containing 0.1% Tween 20 (PBS-T) at 37° C. for 10 min. The organoids fragments on the slide were circled with SuperHT Pap Pen (Biotium, Fremont, CA; Catalog No. 22006). A volume of 300 μL of blocking solution (5% donkey serum in PBS-T) was applied to each slide and incubated at room temperature in a humidified chamber for 1 hour. After removal of the blocking solution, primary antibodies (see table below) diluted in 5% bovine serum albumin (BSA) in PBS-T were incubated overnight at room temperature in a humidified chamber.

| | | Catalog | Dilu- |
|---|---|---|---|
| Reagent | Source | No. | tion |
| Rabbit polyclonal anti-laminin | Abcam | ab11575 | 1:250 |
| Alexa Fluor ™ 594 Phalloidin | Invitrogen | A12381 | 1:250 |
| Lotus Tetragonolobus Lectin | Vector Laboratories | FL-1321-2 | 1:200 |
| (LTL), Fluorescein | | | |
| Mouse monoclonal anti-Arl13b | UC Davis NIH | 75-287 | 1:500 |
| | NeuroMab | | |
| Rabbit polyclonal | Santa Cruz | Sc-7870 | 1:500 |
| anti-E-cadherin | Biotechnology | | |

After three washes, secondary antibodies diluted in 5% BSA in PBS-T were added and incubated for 2 hours in the dark. Then slides were washed three times with PBS-T for 30 min. Slides were air-dried for 5 min and coverslips were mounted with Invitrogen™ Prolong™ Gold Antifade Mountant with DNA Stain DAPI (Thermo Fisher Scientific Catalog No. P36935).

Fluorescent images were obtained using a ZEISS LSM900+ Airyscan microscope (ZEISS Microscopy, Jena, Germany) and analyzed by ZEN lite software (ZEISS Microscopy).

### Results

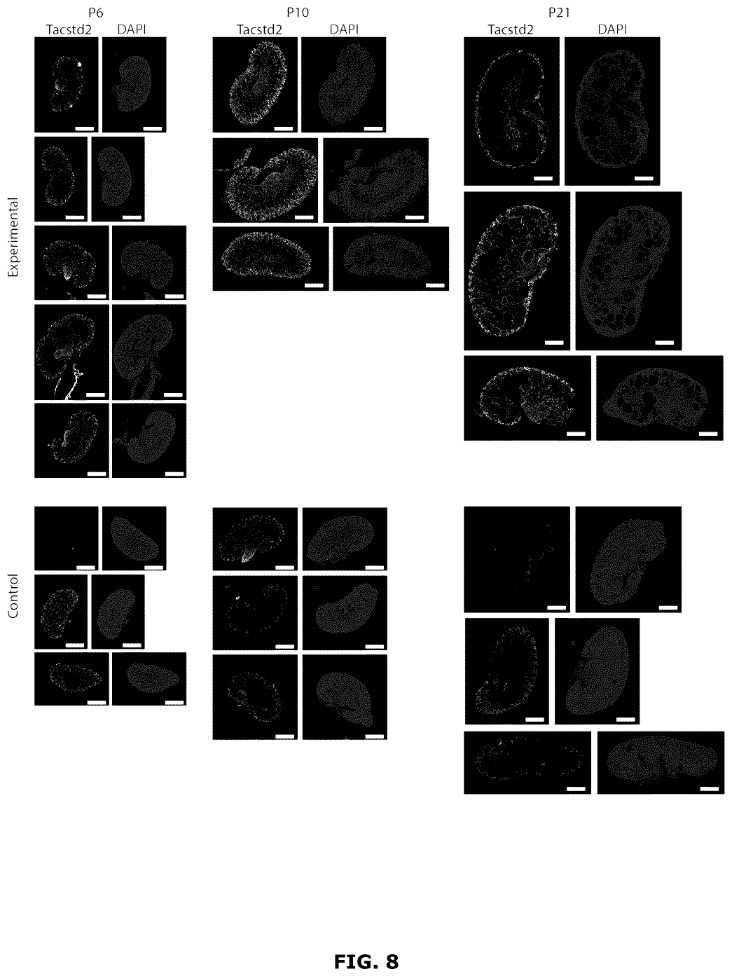

To validate the epithelial expression of Tacstd2 predicted by single-cell RNAseq, kidney tissues collected from the same cohort of animals that were used for RNA isolation in this study (FIGS. 7A, 7B) were immuno-stained. In addition, to see how Tacstd2 responds in more highly cystic kidneys, P21 tissue, collected from another study where Pkd2 was deleted in the early post-natal period by tamoxifen treatment of mothers nursing Pkd2^{flox/flox}:Rosa26-Cre^{ERT} pups (20), was examined (FIGS. 7A, 7B). Anti-Tacstd2 was not found to stain proximal tubules in either control or Pkd2 mutant tissue. The loop of Henle showed only minimal Tacstd2 label in the pre cystic tissue, but loop of Henle-derived cysts showed Tacstd2 label (FIG. 7C). Tacstd2 labeled collecting ducts in both control and experimental kidneys (FIGS. 7C, 7D). While Tacstd2 was found in control collecting ducts, the signal was higher in the experimental collecting ducts at all time points (FIGS. 7A, 7B). When normalized by DAPI signal to represent the total number of cells, Tacstd2 was significantly increased in experimental compared to control at P10 and P21. At P6 the difference did not reach significance (FIG. 7B). All images used for quantification are included in FIG. 8.

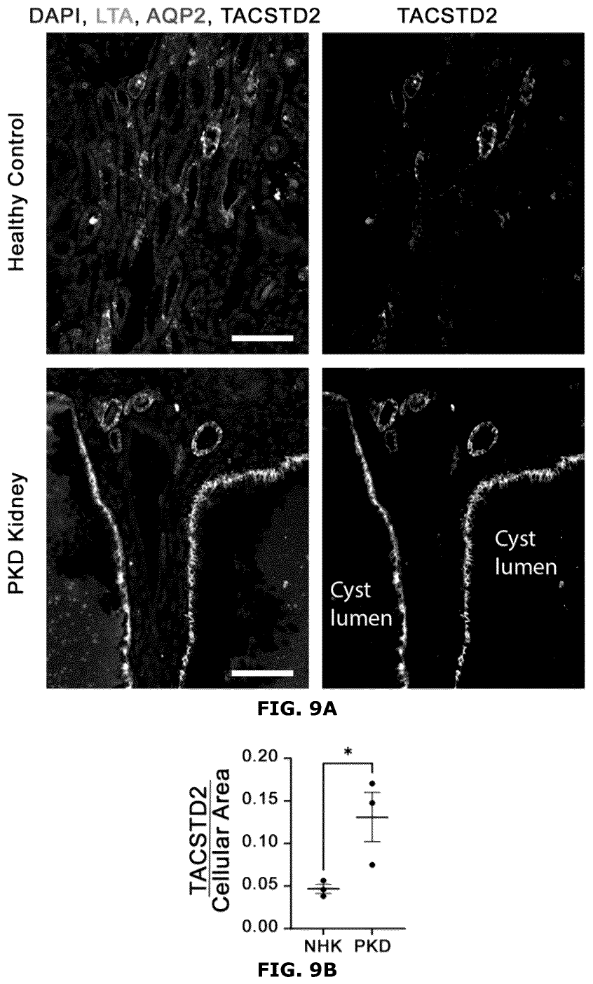

The elevated Tacstd2 expression in the Pkd2 mutant mouse kidneys suggested TACSTD2 expression may also be elevated in human ADPKD samples. Sections of adult male non-cystic and cystic kidneys were obtained. The genotypes were unknown, but the early disease development in these patients was suggestive of PKD1 mutations. Similar to what was observed in the disclosed murine models, TACSTD2 was only faintly detected in control tissues but was strongly observed in the cyst-lining epithelial tissue from cystic patients and overall levels of TACSTD2 signal were higher in the cystic kidneys (FIGS. 9A, 9B).

Since the genotypes of the patient samples were undetermined, the next investigation aimed to determine whether TACSTD2 was elevated by polycystin loss in human kidney organoids with defined genotypes. To do this, control, PKD1^{-/-}, and PKD2^{-/-} IPS cells generated by the PKD Research Resource Consortium were used to generate organoids using a slightly modified Ruiter et al. protocol (21). The resulting kidney organoid model gave rise to nephron components, including the renal proximal tubule and distal convoluted tubule. The proximal tubule had extensive brush borders on the apical surface, marked by Lotus tetragonolobus lectin (LTL; see FIG. 10C) and phalloidin (selectively labels F-actin filaments; see FIG. 10C, second row only). The renal distal tubules were marked by high E-cadherin expression (FIG. 10C, fourth row only). The basement membrane on the basolateral surface of the renal tubules was marked by laminin glycoprotein (FIG. 10C, first, third, and fourth rows). Primary cilia, which are present on the apical surface of the tubule epithelial cells, were marked by enrichment of Arl13b GTPase (FIG. 10C, first through third rows).

This protocol produced robust cysts from both mutant cell lines, although the PKD1^{-/-}-derived organoids were more extensively cystic (FIGS. 10D, 10E). Control organoids showed little staining with TACSTD2 antibodies while PKD1^{-/-} and PKD2^{-/-} organoids showed robust TACSTD2 label of the tubules and cysts (FIG. 10F). The very large cysts that developed when PKD1^{-/-} was mutated were strongly stained (FIGS. 10F, 10G). TACSTD2 stained the whole cell with highest concentration at the cell surface (FIG. 10F). From this analysis, it was concluded that TACSTD2 levels increased with loss of either PKD1 or PKD2.

### Example 5

### Investigating the Response of PKD Organoids to Anti-TACSTD2 Antibody-Drug Conjugate Sacituzumab Govitecan

### Materials and Methods

Treatment of PKD Organoids with Sacituzumab Govitecan

Kidney organoids were differentiated, as described in Example 4, from control, PKD1^{-/-}, and PKD2^{-/-} IPS cells. Cysts developed after four weeks. Kidney organoids were collected four weeks post organoid formation and transferred to 24-well ultra-low attachment plate at a density of 1 organoid per well. On day one, control, cystic PKD14, and cystic PKD2^{-/-} organoids were suspended in wells containing either 10 nM sacituzumab govitecan (IMMU-132; Selleck Chemicals, Houston, TX; Catalog No. E2841) in STEMdiff™ APEL™2 medium or only STEMdiff™ APEL™2 medium. Organoids were washed with DPBS, and the media was replaced with fresh media containing 10 nM of sacituzumab govitecan and APEL2 only every four days. Organoids were imaged every two to three days.

### Data Analysis

Kidney organoids were imaged via light microscopy dissecting scope, and their area was determined via ImageJ (NIH). Organoid area at a given timepoint was normalized to the corresponding day 1 area, and the following images were then assessed for percent change relative to day 1. Prism GraphPad was used to construct line graphs depicting organoid growth and area reduction without treatment and when given 10 nM of sacituzumab govitecan. The change in organoid area was recorded as a percent increase relative to day 1 measured area. For PKD1^{-/-} and PKD2^{-/-} kidney organoids, cysts contributed to a majority of the recorded area; therefore, variations in area percent change largely account for variances observed in the cyst area.

### Results

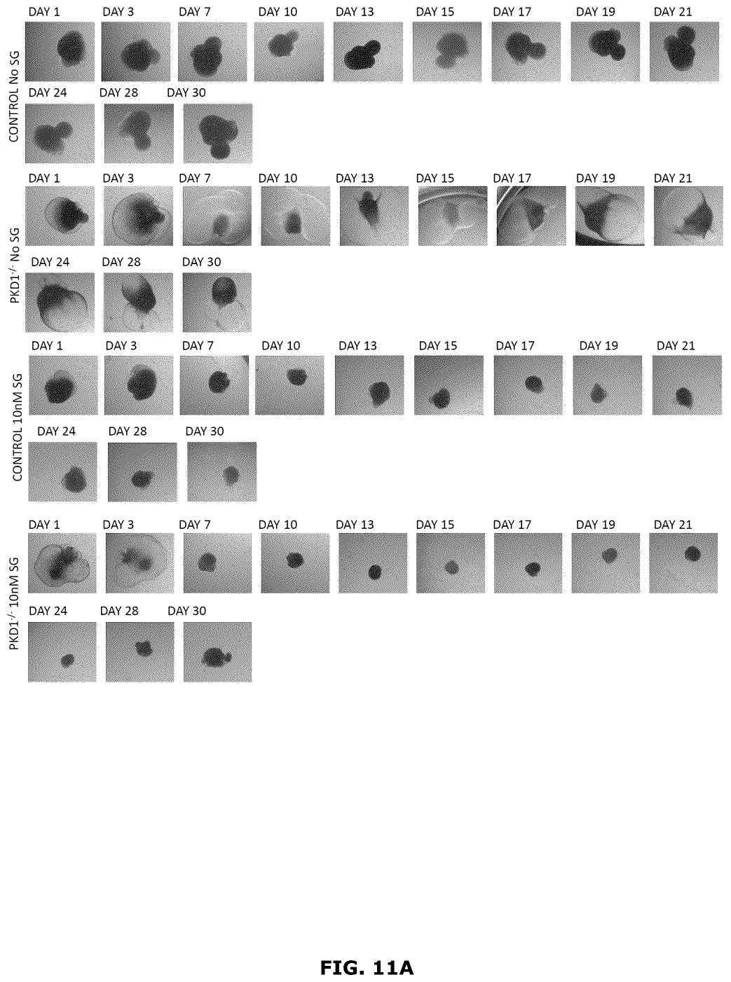

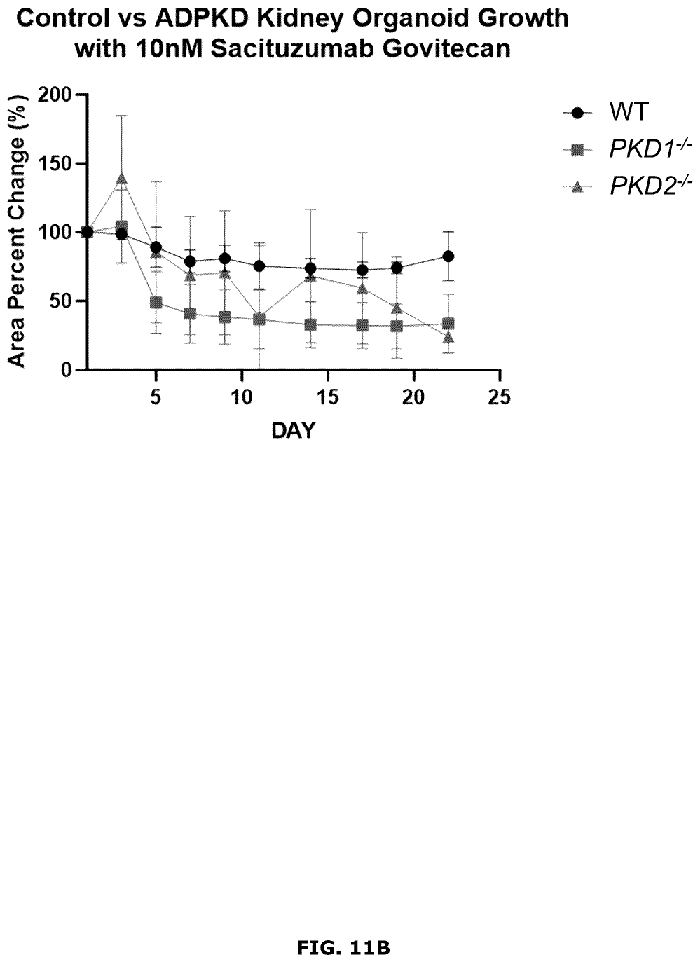

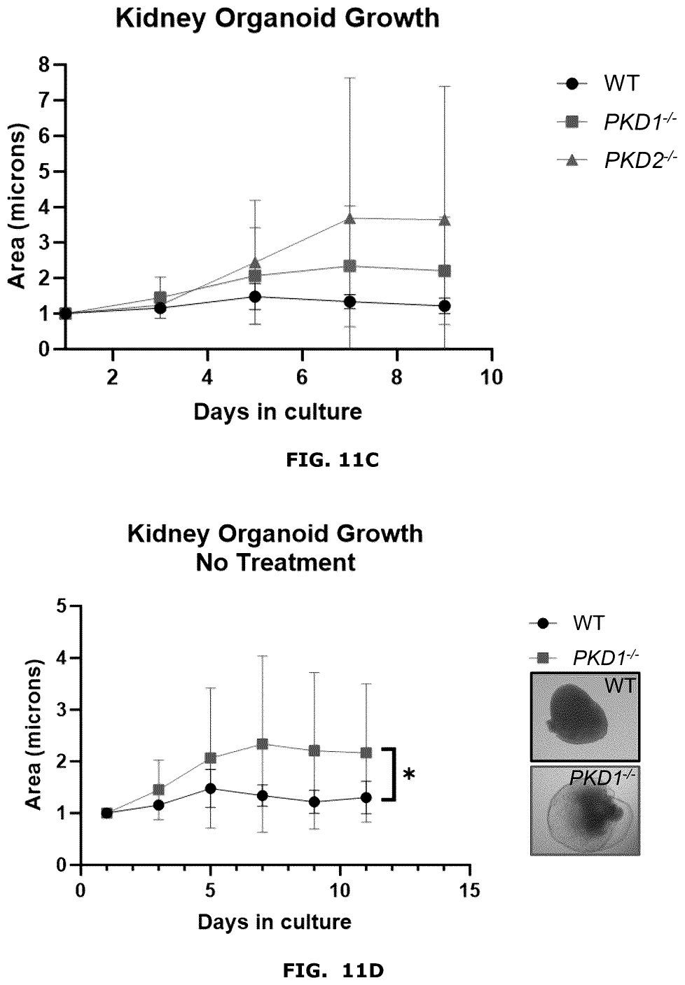

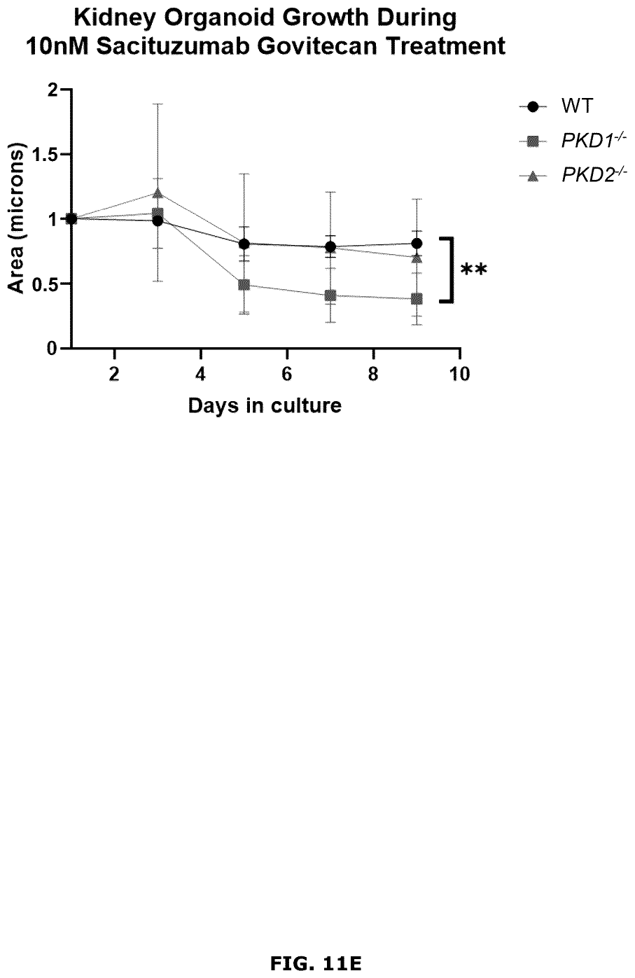

Untreated kidney organoids exhibited continued growth while cultured. Control (untreated) organoids grew by 9% by Day 3, 14% by Day 10, and 17% by Day 15 (FIGS. 11A, 11C, 11E). PKD1^{-/-} untreated kidney organoids grew by 111% by Day 3, 328% by Day 10, and 721% by Day 15 (FIGS. 11A, 11C, 11E). PKD2^{-/-} untreated kidney organoids grew by 32% by Day 3, 345% by Day 10, and 1,582% by Day 15. Cystic kidney organoids exhibited greater growth rates than control kidney organoids. Additionally, beyond Day 15, the cystic area for PKD1^{-/-} and PKD2^{-/-} organoids began to decrease, likely due to the well area being insufficient to accommodate large cyst area and volume, particularly for PKD2^{-/-} kidney organoids.

Treated control organoids exhibited a modest decrease in area percent change of −2% by Day 3 (FIGS. 11A, 11B, 11D). PKD1^{-/-} organoids exhibited a 4% increase in area percent change (FIGS. 11A, 11B, 11D), while PKD2^{-/-} organoids exhibited a 40% increase (FIG. 11B). By Day 7, treated PKD1^{-/-} and PKD2^{-/-} kidney organoids displayed a reduced area percent change of 59% and 31%, respectively. Control kidney organoids exhibited a 21% reduction in area percent change. The reduction in cyst area for PKD1^{-/-} and PKD2^{-/-} continued, as by Day 11 the reduction in area percent change was 63% and 61%, respectively. By Day 11, control treated organoids exhibited a 25% reduction in area percent change. These variances were maintained during the treated duration, with control organoids reaching a 17% reduction in area percent change, PKD1^{-/-} cystic organoids reaching a 66% reduction in area, and PKD2^{-/-} cystic organoids reaching a 76% reduction in area.

By investigating the pre-cystic stage of disease progression in an ADPKD mouse model, Tacstd2 was identified as a gene that is upregulated during the cyst initiation event and likely promotes abnormal proliferation following this event; subsequent work confirmed the validity of TACSTD2 as a cyst initiation candidate in human tissue and organoid models as well. Because TACSTD2 was determined to be upregulated not only at cyst initiation, but persistently throughout cyst development and growth as well, TACSTD2—a transmembrane protein with a significant extracellular domain—was identified as a potential target for antibody-directed therapy for ADPKD. An antibody-conjugated drug that targets TACSTD2-overexpressing cells, sacituzumab govitecan, was tested. Sacituzumab govitecan targets, but does not inhibit, TACSTD2, and is FDA-approved as a treatment for some forms of aggressive cancer. No adjustment to the starting dose is required when administering sacituzumab govitecan to patients with mild or moderate renal impairment, suggesting that this antibody-drug conjugate (ADC) may be suitable for ADPKD patients. Renal elimination of SN-38, the small molecule moiety of sacituzumab govitecan, is minimal in patients with renal impairment or end-stage renal disease (i.e., patients with creatinine clearance (CrCl) of less than <15 mL/min); however, no pharmacokinetic studies of sacituzumab govitecan have been conducted (57). The effects of sacituzumab govitecan and free SN-38 on patients with moderate or severe hepatic impairment have not been studied. Pharmacokinetic analyses in patients treated with sacituzumab govitecan (n=527) did not identify an effect of age, race, or mild renal impairment on the pharmacokinetics of sacituzumab govitecan (58).

In the disclosed human kidney PKD knockout organoid model, treatment with sacituzumab govitecan proved to have restorative effects by eliminating cysts, drastically reducing cyst growth rate, preventing expansion, and preserving intact renal tubule structure. These data suggest that TACSTD2-targeted treatment with an antibody-drug conjugate in human ADPKD patients would be able to restore, or at least significantly improve, normal kidney architecture and renal function.

### REFERENCES

1. Lanktree, M. B., Haghighi, A., Guiard, E., Iliuta, I. A., Song, X., Harris, P. C., Paterson, A. D., and Pei, Y. (2018) Prevalence Estimates of Polycystic Kidney and Liver Disease by Population Sequencing. J Am Soc Nephrol 29, 2593-2600
2. Iglesias, C. G., Torres, V. E., Offord, K. P., Holley, K. E., Beard, C. M., and Kurland, L. T. (1983) Epidemiology of adult polycystic kidney disease, Olmsted County, Minnesota: 1935-1980. Am J Kidney Dis 2, 630-639
3. Dalgaard, O. Z. (1957) Bilateral polycystic disease of the kidneys; a follow-up of 284 patients and their families. Dan Med Bull 4, 128-133
4. Torres, V. E., Chapman, A. B., Devuyst, O., Gansevoort, R. T., Grantham, J. J., Higashihara, E., Perrone, R. D., Krasa, H. B., Ouyang, J., Czerwiec, F. S., and Investigators, T. T. (2012) Tolvaptan in patients with autosomal dominant polycystic kidney disease. N Engl J Med 367, 2407-2418
5. Torres, V. E., Devuyst, O., Chapman, A. B., Gansevoort, R. T., Perrone, R. D., Ouyang, J., Blais, J. D., Czerwiec, F. S., Sergeyeva, O., and Investigators, R. T. (2017) Rationale and Design of a Clinical Trial Investigating Tolvaptan Safety and Efficacy in Autosomal Dominant Polycystic Kidney Disease. Am J Nephrol 45, 257-266
6. McEwan, P., Bennett Wilton, H., Ong, A. C. M., Orskov, B., Sandford, R., Scolari, F., Cabrera, M. V., Walz, G., O'Reilly, K., and Robinson, P. (2018) A model to predict disease progression in patients with autosomal dominant polycystic kidney disease (ADPKD): the ADPKD Outcomes Model. BMC Nephrol 19, 37
7. Cornec-Le Gall, E., Alam, A., and Perrone, R. D. (2019) Autosomal dominant polycystic kidney disease. Lancet 393, 919-935
8. Oberdhan, D., Schaefer, F., Cole, J. C., Palsgrove, A. C., Dandurand, A., and Guay-Woodford, L. (2022) Polycystic Kidney Disease-Related Disease Burden in Adolescents With Autosomal Dominant Polycystic Kidney Disease: An International Qualitative Study. Kidney Med 4, 100415
9. Fick-Brosnahan, G. M., Tran, Z. V., Johnson, A. M., Strain, J. D., and Gabow, P. A. (2001) Progression of autosomal-dominant polycystic kidney disease in children. Kidney Int 59, 1654-1662
10. Bardia, A., Hurvitz, S. A., Tolaney, S. M., Loirat, D., Punie, K., Oliveira, M., Brufsky, A., Sardesai, S. D., Kalinsky, K., Zelnak, A. B., Weaver, R., Traina, T., Dalenc, F., Aftimos, P., Lynce, F., Diab, S., Cortes, J., O'Shaughnessy, J., Dieras, V., Ferrario, C., Schmid, P., Carey, L. A., Gianni, L., Piccart, M. J., Loibl, S., Goldenberg, D. M., Hong, Q., Olivo, M. S., Itri, L. M., Rugo, H. S., and Investigators, A. C. T. (2021) Sacituzumab Govitecan in Metastatic Triple-Negative Breast Cancer. N Engl J Med 384, 1529-1541
11. Garcia-Gonzalez, M. A., Outeda, P., Zhou, Q., Zhou, F., Menezes, L. F., Qian, F., Huso, D. L., Germino, G. G., Piontek, K. B., and Watnick, T. (2010) Pkd1 and Pkd2 are required for normal placental development. PLOS One 5
12. Song, G., Zhang, H., Chen, C., Gong, L., Chen, B., Zhao, S., Shi, J., Xu, J., and Ye, Z. (2017) miR-551b regulates epithelial-mesenchymal transition and metastasis of gastric cancer by inhibiting ERBB4 expression. Oncotarget 8, 45725-45735
13. Wei, Z., Liu, Y., Wang, Y., Zhang, Y., Luo, Q., Man, X., Wei, F., and Yu, X. (2016) Downregulation of Foxo3 and TRIM31 by miR-551b in side population promotes cell proliferation, invasion, and drug resistance of ovarian cancer. Med Oncol 33, 126
14. Woo, Y. M., Kim, D. Y., Koo, N. J., Kim, Y. M., Lee, S., Ko, J. Y., Shin, Y., Kim, B. H., Mun, H., Choi, S., Lee, E. J., Shin, J. O., Park, E. Y., Bok, J., and Park, J. H. (2017) Profiling of miRNAs and target genes related to cystogenesis in ADPKD mouse models. Sci Rep 7, 14151
15. Harris, P. C., and Torres, V. E. (2009) Polycystic kidney disease. Annu Rev Med 60, 321-337
16. Ransick, A., Lindström, N. O., Liu, J., Zhu, Q., Guo, J.-J., Alvarado, G. F., Kim, A. D., Black, H. G., Kim, J., and McMahon, A. P. (2019) Single-Cell Profiling Reveals Sex, Lineage, and Regional Diversity in the Mouse Kidney. Developmental Cell 51, 399-413.e397
17. Chen, L., Clark, J. Z., Nelson, J. W., Kaissling, B., Ellison, D. H., and Knepper, M. A. (2019) Renal-Tubule Epithelial Cell Nomenclature for Single-Cell RNA-Sequencing Studies. J Am Soc Nephrol 30, 1358-1364
18. Devuyst, O., Burrow, C. R., Smith, B. L., Agre, P., Knepper, M. A., and Wilson, P. D. (1996) Expression of aquaporins-1 and -2 during nephrogenesis and in autosomal dominant polycystic kidney disease. Am J Physiol 271, F169-183
19. Muto, Y., Dixon, E. E., Yoshimura, Y., Wu, H., Omachi, K., Ledru, N., Wilson, P. C., King, A. J., Eric Olson, N., Gunawan, M. G., Kuo, J. J., Cox, J. H., Miner, J. H., Seliger, S. L., Woodward, O. M., Welling, P. A., Watnick, T. J., and Humphreys, B. D. (2022) Defining cellular complexity in human autosomal dominant polycystic kidney disease by multimodal single cell analysis. Nature Communications 13
20. Smith, A. O., Jonassen, J. A., Preval, K. M., Davis, R. J., and Pazour, G. J. (2021) c-Jun N-terminal kinase (JNK) signaling contributes to cystic burden in polycystic kidney disease. PLOS Genet 17, e1009711
21. Ruiter, F. A. A., Morgan, F. L. C., Roumans, N., Schumacher, A., Slaats, G. G., Moroni, L., LaPointe, V. L. S., and Baker, M. B. (2022) Soft, Dynamic Hydrogel Confinement Improves Kidney Organoid Lumen Morphology and Reduces Epithelial-Mesenchymal Transition in Culture. Adv Sci (Weinh) 9, e2200543
22. Kunnen, S. J., Malas, T. B., Formica, C., Leonhard, W. N., t Hoen, P. A. C., and Peters, D. J. M. (2018) Comparative transcriptomics of shear stress treated Pkd1 (−/−) cells and pre-cystic kidneys reveals pathways involved in early polycystic kidney disease. Biomed Pharmacother 108, 1123-1134
23. Zhang, C., Balbo, B., Ma, M., Zhao, J., Tian, X., Kluger, Y., and Somlo, S. (2021) Cyclin-Dependent Kinase 1 Activity Is a Driver of Cyst Growth in Polycystic Kidney Disease. J Am Soc Nephrol 32, 41-51
24. Lipinski, M., Parks, D. R., Rouse, R. V., and Herzenberg, L. A. (1981) Human trophoblast cell-surface antigens defined by monoclonal antibodies. Proc Natl Acad Sci USA 78, 5147-5150
25. Shvartsur, A., and Bonavida, B. (2015) Trop2 and its overexpression in cancers: regulation and clinical/therapeutic implications. Genes Cancer 6, 84-105
26. Trerotola, M., Cantanelli, P., Guerra, E., Tripaldi, R., Aloisi, A. L., Bonasera, V., Lattanzio, R., de Lange, R., Weidle, U. H., Piantelli, M., and Alberti, S. (2013) Upregulation of Trop-2 quantitatively stimulates human cancer growth. Oncogene 32, 222-233
27. Qiu, S., Zhang, J., Wang, Z., Lan, H., Hou, J., Zhang, N., Wang, X., and Lu, H. (2023) Targeting Trop-2 in cancer: Recent research progress and clinical application. Biochim Biophys Acta Rev Cancer 1878, 188902
28. Wang, J., Zhang, K., Grabowska, D., Li, A., Dong, Y., Day, R., Humphrey, P., Lewis, J., Kladney, R. D., Arbeit, J. M., Weber, J. D., Chung, C. H., and Michel, L. S. (2011) Loss of Trop2 promotes carcinogenesis and features of epithelial to mesenchymal transition in squamous cell carcinoma. Mol Cancer Res 9, 1686-1695
29. Kaza, H., Barik, M. R., Reddy, M. M., Mittal, R., and Das, S. (2017) Gelatinous drop-like corneal dystrophy: a review. Br J Ophthalmol 101, 10-15
30. Nakatsukasa, M., Kawasaki, S., Yamasaki, K., Fukuoka, H., Matsuda, A., Tsujikawa, M., Tanioka, H., Nagata-Takaoka, M., Hamuro, J., and Kinoshita, S. (2010) Tumor-associated calcium signal transducer 2 is required for the proper subcellular localization of claudin 1 and 7: implications in the pathogenesis of gelatinous drop-like corneal dystrophy. Am J Pathol 177, 1344-1355
31. Ripani, E., Sacchetti, A., Corda, D., and Alberti, S. (1998) Human Trop-2 is a tumor-associated calcium signal transducer. Int J Cancer 76, 671-676
32. Guerra, E., Trerotola, M., Tripaldi, R., Aloisi, A. L., Simeone, P., Sacchetti, A., Relli, V., D'Amore, A., La Sorda, R., Lattanzio, R., Piantelli, M., and Alberti, S. (2016) Trop-2 Induces Tumor Growth Through AKT and Determines Sensitivity to AKT Inhibitors. Clin Cancer Res 22, 4197-4205
33. Lenart, S., Lenart, P., Smarda, J., Remsik, J., Soucek, K., and Benes, P. (2020) Trop2: Jack of All Trades, Master of None. Cancers (Basel) 12
34. Zhao, W., Jia, L., Kuai, X., Tang, Q., Huang, X., Yang, T., Qiu, Z., Zhu, J., Huang, J., Huang, W., and Feng, Z. (2019) The role and molecular mechanism of Trop2 induced epithelial-mesenchymal transition through mediated beta-catenin in gastric cancer. Cancer Med 8, 1135-1147
35. Saadi-Kheddouci, S., Berrebi, D., Romagnolo, B., Cluzeaud, F., Peuchmaur, M., Kahn, A., Vandewalle, A., and Perret, C. (2001) Early development of polycystic kidney disease in transgenic mice expressing an activated mutant of the beta-catenin gene. Oncogene 20, 5972-5981
36. Hayashi, S., and McMahon, A. P. (2002) Efficient recombination in diverse tissues by a tamoxifen-inducible form of Cre: a tool for temporally regulated gene activation/inactivation in the mouse. Dev Biol 244, 305-318
37. Badea, T. C., Wang, Y., and Nathans, J. (2003) A noninvasive genetic/pharmacologic strategy for visualizing cell morphology and clonal relationships in the mouse. J Neurosci 23, 2314-2322
38. Sadate-Ngatchou, P. I., Payne, C. J., Dearth, A. T., and Braun, R. E. (2008) Cre recombinase activity specific to postnatal, premeiotic male germ cells in transgenic mice. Genesis 46, 738-742
39. Jonassen, J. A., San Agustin, J., Follit, J. A., and Pazour, G. J. (2008) Deletion of IFT20 in the mouse kidney causes misorientation of the mitotic spindle and cystic kidney disease. J Cell Biol 183, 377-384
40. Yukselen, O., Turkyilmaz, O., Ozturk, A. R., Garber, M., and Kucukural, A. (2020) DolphinNext: a distributed data processing platform for high throughput genomics. BMC Genomics 21, 310
41. Bray, N. L., Pimentel, H., Melsted, P., and Pachter, L. (2016) Near-optimal probabilistic RNA-seq quantification. Nat Biotechnol 34, 525-527
42. Love, M. I., Huber, W., and Anders, S. (2014) Moderated estimation of fold change and dispersion for RNA-seq data with DESeq2. Genome Biol 15, 550
43. Chen, Y., Chen, Y., Shi, C., Huang, Z., Zhang, Y., Li, S., Li, Y., Ye, J., Yu, C., Li, Z., Zhang, X., Wang, J., Yang, H., Fang, L., and Chen, Q. (2018) SOAPnuke: a MapReduce acceleration-supported software for integrated quality control and preprocessing of high-throughput sequencing data. Gigascience 7, 1-6
44. Muller, S., Rycak, L., Winter, P., Kahl, G., Koch, I., and Rotter, B. (2013) omiRas: a Web server for differential expression analysis of miRNAs derived from small RNA-Seq data. Bioinformatics 29, 2651-2652
45. Langmead, B., Trapnell, C., Pop, M., and Salzberg, S. L. (2009) Ultrafast and memory-efficient alignment of short DNA sequences to the human genome. Genome Biol 10, R25
46. Friedlander, M. R., Mackowiak, S. D., Li, N., Chen, W., and Rajewsky, N. (2012) miRDeep2 accurately identifies known and hundreds of novel microRNA genes in seven animal clades. Nucleic Acids Res 40, 37-52
47. Anders, S., and Huber, W. (2010) Differential expression analysis for sequence count data. Genome Biol 11, R106
48. Stuart, T., Butler, A., Hoffman, P., Hafemeister, C., Papalexi, E., Mauck, W. M., 3rd, Hao, Y., Stoeckius, M., Smibert, P., and Satija, R. (2019) Comprehensive Integration of Single-Cell Data. Cell 177, 1888-1902 e1821
49. Edgar, R., Domrachev, M., and Lash, A. E. (2002) Gene Expression Omnibus: NCBI gene expression and hybridization array data repository. Nucleic Acids Res 30, 207-210
50. Olson, R., Hopp, K., Wells, H., Smith, J., Furtado, J., Constans, M., Escobar, D., Geurts A., Torres, V., and Harris P., (2019) Synergistic Genetic Interactions between Pkhd1 and Pkd1 Result in an ARPKD-Like Phenotype in Murine Models. J Am Soc Nephrol. 30 (11): 2113-2127
51. Mekahli D., Guay-Woodford, L., Cadnapaphornchai, M., Greenbaum, L., Litwin, M., Seeman, T., Dandurand, A., Shi, L., Sikes, K., Shoaf, S., and Schaefer, F. (2023) Tolvaptan for Children and Adolescents with Autosomal Dominant Polycystic Kidney Disease: Randomized Controlled Trial. Clin J Am Soc Nephrol. 18 (1): 36-46
52. Erickson, K., Chertow, G., and Goldhaber-Fiebert, J. (2013) Cost-effectiveness of tolvaptan in autosomal dominant polycystic kidney disease. Ann Intern Med. 159 (6): 382-9
53. Gilbert, R., Evans, H., Olalekan, K., Nagra, A., Haq, M., and Griffiths, M. (2017) Tolvaptan treatment for severe neonatal autosomal-dominant polycystic kidney disease. Pediatr Nephrol. 32 (5): 893-896
54. Lioudis, M., Zhou, X., Davenport, E., Nunna, S., Krasa, H., Oberdhan, D., and Fernandes, A. (2023) Effects of tolvaptan discontinuation in patients with autosomal dominant polycystic kidney disease: a post hoc pooled analysis. BMC Nephrol. 24 (1): 182
55. Lee, E., Valencia, T., Allerson, C., Schairer, A., Flaten, A., Yheskel, M., Kersjes, K., Li, J., Gatto, S., Takhar, M., Lockton, S., Pavlicek, A., Kim, M., Chu, T., Soriano, R., Davis, S., Androsavich, J., Sarwary, S., Owen, T., Kaplan, J., Liu, K., Jang, G., Neben, S., Bentley, P., Wright, T., and Patel, V. (2019) Discovery and preclinical evaluation of anti-miR-17 oligonucleotide RGLS4326 for the treatment of polycystic kidney disease. Nat Commun. 10(1):4148.
56. Rugo, H., Bardia, A., Tolaney, S., Arteaga, C., Cortes, J., Sohn, J., Marmé, F., Hong, Q., Delaney, R., Hafeez, A., André, F., and Schmid, P. (2020) TROPICS-02: A Phase III study investigating sacituzumab govitecan in the treatment of HR+/HER2− metastatic breast cancer. Future Oncol. 16 (12): 705-715.
57. Syed, Y. (2020) Sacituzumab Govitecan: First Approval. Drugs. 80 (10): 1019-1025.
58. Michaleas, S., Moreno Oliver, A., Mueller-Berghaus, J., Sarac, S., van der Elst, M., Müller-Egert, S., Zander, H., Enzmann, H., and Pignatti, F. (2022) The European Medicines Agency review of sacituzumab govitecan for the treatment of triple-negative breast cancer. ESMO Open. 7 (3): 100497.
59. Shastry, M., Jacob, S., Rugo, H., and Hamilton, E. (2022) Antibody-drug conjugates targeting TROP-2: Clinical development in metastatic breast cancer. Breast. 66:169-177.
60. Calvet, J. (2015) The Role of Calcium and Cyclic AMP in PKD. In: Li X, editor. Polycystic Kidney Disease. Brisbane, AUS: Codon Publications. Chapter 8.
61. Pavone, G., Motta, L., Martorana, F., Motta, G., and Vigneri, P. (2021) A New Kid on the Block: Sacituzumab Govitecan for the Treatment of Breast Cancer and Other Solid Tumors. Molecules 26:7294.
62. Strop, P., Tran, T. T., Dorywalska, M., Delaria, K., Dushin, R., Wong, O. K., Ho, W. H., Zhou, D., Wu, A., Kraynov, E., Aschenbrenner, L., Han, B., O'Donnell, CJ., Pons, J., Rajpal, A., Shelton, D. L., Liu, S. H. RN927C, a Site-Specific Trop-2 Antibody-Drug Conjugate (ADC) with Enhanced Stability, Is Highly Efficacious in Preclinical Solid Tumor Models. Mol Cancer Ther. 2016 November; 15(11):2698-2708.
63. Kolberg, L., Raudvere, U., Kuzmin, I., Adler, P., Vilo, J., Peterson, H. g: Profiler-interoperable web service for functional enrichment analysis and gene identifier mapping (2023 update). Nucleic Acids Res. 2023; 51 (W1): W207-W212.
64. Pretorius, D. H., Lee, M. E., Manco-Johnson, M. L., Weingast, G. R., Sedman, A. B., Gabow, P. A. Diagnosis of autosomal dominant polycystic kidney disease in utero and in the young infant. J Ultrasound Med. 1987; 6 (5): 249-55.
65. Fick, G. M., Johnson, A. M., Strain, J. D., Kimberling, W. J., Kumar, S., Manco-Johnson, M. L., et al. Characteristics of very early onset autosomal dominant polycystic kidney disease. J Am Soc Nephrol. 1993; 3 (12): 1863-70.
66. Grantham, J. J., Cook, L. T., Wetzel, L. H., Cadnapaphornchai, M. A., Bae, K. T. Evidence of extraordinary growth in the progressive enlargement of renal cysts. Clin J Am Soc Nephrol. 2010; 5 (5): 889-96.
67. De Rechter, S., Breysem, L., Mekahli, D. Is Autosomal Dominant Polycystic Kidney Disease Becoming a Pediatric Disorder? Front Pediatr. 2017; 5:272.
68. Liu, B., Singh, K., Gong, S., Canakci, M., Osborne, B. A., Thayumanavan, S. Protein-Antibody Conjugates (PACs): A Plug-and-Play Strategy for Covalent Conjugation and Targeted Intracellular Delivery of Pristine Proteins. Angew Chem Int Ed Engl. 2021 Jun. 1; 60(23):12813-12818. doi: 10.1002/anie.202103106.

### CONCLUSION

The T teachings of all patents, published applications, and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of treating a subject in need thereof, comprising administering to the subject a therapeutically effective amount of an antibody-therapeutic conjugate, wherein the antibody-therapeutic conjugate comprises an antibody that binds a marker of cystic epithelium, and a therapeutic drug; and wherein the subject has or is predisposed to have a polycystic kidney disease (PKD).

2. The method of claim 1, wherein the antibody comprises a whole antibody or an antigen-binding fragment thereof.

3. The method of claim 1, wherein the marker of cystic epithelium comprises tumor associated calcium signal transducer 2 (TACSTD2).

4. The method of claim 1, wherein the antibody is selected from the group comprising sacituzumab, datopotamab, and humanized anti-TACSTD2 antibody RN926.

5. The method of claim 1, wherein the antibody binds TACSTD2 and an additional antigen.

6. The method of claim 1, wherein the therapeutic drug comprises a cytotoxic drug, a cytostatic drug, a gene therapy or a combination thereof.

7. The method of claim 5, wherein the gene therapy provides a means for gene editing, gene augmentation, gene replacement, gene addition or a combination thereof.

8. The method of claim 6, wherein the gene therapy targets at least one gene selected from polycystin 1, transient receptor potential channel interacting (PKD1), polycystin 2, transient receptor potential cation channel (PKD2), and tumor associated calcium signal transducer 2 (TACSTD2).

9. The method of claim 6, wherein the gene therapy comprises an anti-sense oligonucleotide (ASO), a small interfering RNA (siRNA), a viral vector, a non-viral delivery system or a combination thereof.

10. The method of claim 2, wherein the therapeutic drug comprises a topoisomerase I inhibitor, a tubulin polymerization inhibitor, a vasopressin receptor 2 antagonist, an mTOR inhibitor or a combination thereof.

11. The method of claim 2, wherein the therapeutic drug comprises a topoisomerase I inhibitor SN38, deruxtecan, tubulin polymerization inhibitor Aur0101, tolvaptan, rapamycin or a combination thereof.

12. The method of claim 1, wherein the antibody-therapeutic conjugate comprises sacituzumab govitecan (hRS7-SN38), datopotamab deruxtecan (Dato-DXd), PF-06664178 (RN926-Aur0101) or a combination thereof.

13. The method of claim 1, wherein the polycystic kidney disease is autosomal dominant polycystic kidney disease (ADPKD) or autosomal recessive polycystic disease (ARPKD).

14. The method of claim 13, wherein the ADPKD is associated with PKD1 or PKD2.

15. The method of claim 1, wherein the method further comprises administering a therapeutically effective amount of an additional therapeutic agent or a prophylactic agent to the subject.

16. The method of claim 15, wherein the additional therapeutic agent or the prophylactic agent is tolvaptan.

17. The method of claim 1, wherein the method further comprises administering an additional treatment to the subject.

18. A method of evaluating a biological response to an antibody-therapeutic conjugate, the method comprising:
a) exposing:
i) a first kidney organoid to the antibody-therapeutic conjugate without exposing a second kidney organoid to the antibody-therapeutic conjugate; or
ii) a first population of stem cells to the antibody-therapeutic conjugate, thereby producing a population of exposed stem cells, without exposing a second population of cells to the antibody-therapeutic conjugate, and differentiating the population of exposed stem cells into a first kidney organoid and differentiating the second population of stem cells into a second kidney organoid; and
b) comparing the first kidney organoid and the second kidney organoid, thereby evaluating the biological response.

19. The method of claim 18, wherein the stem cells are induced pluripotent stem (IPS) cells generated from a biological sample from a subject.

20. A method of reducing or ablating cystic kidney tissue in a subject in need thereof, the method comprising administering to the subject a therapeutically effective amount of an antibody-therapeutic conjugate, wherein said antibody-therapeutic conjugate comprises:
a monospecific antibody or antigen-binding fragment thereof that binds TACSTD2 or a bispecific antibody or antigen-binding fragment thereof that binds TACSTD2 and a second antigen, and
a topoisomerase I inhibitor, a tubulin polymerization inhibitor, a vasopressin receptor 2 antagonist, an mTOR inhibitor or a combination thereof.


SEQUENCE LISTING (4 sequences)

SEQ ID NO: 1 - DNA, 23 bp, Homo sapiens
  source 1..23: mol_type = genomic DNA
ccggaggcaacagtggagcatgt

SEQ ID NO: 2 - DNA, 23 bp, Homo sapiens
  source 1..23: mol_type = genomic DNA
gcacgggtgggggcggcttcctc

SEQ ID NO: 3 - DNA, 23 bp, Homo sapiens
  source 1..23: mol_type = genomic DNA
gccaattttgtttcccataagcc

SEQ ID NO: 4 - DNA, 20 bp, Homo sapiens
  source 1..20: mol_type = genomic DNA
gcaaagcaggtcctgtcgat